(19) United States
(12) Patent Application Publication
(10) Pub. No.: US 2026/0242359 A1
(43) Pub. Date: Aug. 20, 2026

(54) IRHOM2 INHIBITORS AND USES THEREOF

(71) Applicants: NEW YORK SOCIETY FOR THE RELIEF OF THE RUPTURED AND CRIPPLED, MAINTAING THE HOSPITAL FOR SPECIAL, New York, NY (US); SciRhom GmbH, Planegg (DE)

(72) Inventors: Carl P. Blobel, Eastchester, NY (US); Matthias Schneider, Neufarn (DE); Kerstin Selle, Planegg (DE)

(21) Appl. No.: 19/471,072

(22) PCT Filed: Apr. 4, 2024
(86) PCT No.: PCT/US2024/023027

§ 371 Date: Sep. 30, 2025

Related U.S. Application Data
(60) Provisional application No. 63/494,375, filed on Apr. 5, 2023.

(51) Int. Cl. C07D 403/06; A61K 31/427; A61K 31/445; A61K 31/4985; A61K 31/515; A61P 7/04; A61P 25/00; A61P 25/28; A61P 29/00; A61P 37/00; A61P 37/06; C07D 211/18; C07D 417/12; C07D 498/14

(52) U.S. Cl. CPC C07D 403/06; A61K 31/427; A61K 31/445; A61K 31/4985; A61K 31/515; A61P 7/04; A61P 25/00; A61P 25/28; A61P 29/00; A61P 37/00; A61P 37/06; C07D 211/18; C07D 417/12; C07D 498/14

(57) ABSTRACT

The present application is directed to inhibitors of iRhom2/ADAM17 activity that are useful in the treatment of various diseases.

### CROSS-REFERENCE TO RELATED APPLICATIONS

The present application claims the benefit of U.S. Provisional Application No. 63/494,375, filed Apr. 5, 2023, which is incorporated herein by reference in its entirety.

### SEQUENCE LISTING

This application contains a Sequence Listing that has been submitted electronically as an XML file named 27601-0086WO1_SL_ST26.xml. The XML file, created on Apr. 3, 2024, is 10,084 bytes in size. The material in the XML file is hereby incorporated by reference in its entirety.

### FIELD OF THE INVENTION

The present application is directed to inhibitors of iRhom2/ADAM17 activity that are useful in the treatment of various diseases.

### BACKGROUND OF THE INVENTION

EGFR (epidermal growth factor receptor) exists on the cell surface and is activated by binding of its specific ligands, including epidermal growth factor and transforming growth factor α (TGFα). Upon activation by its growth factor ligands, EGFR undergoes a transition from an inactive monomeric form to an active homodimer (Yosef Yarden and Joseph Schlessinger (1987), “Epidermal Growth-Factor Induces Rapid, Reversible Aggregation of the Purified Epidermal Growth-Factor Receptor”, Biochemistry 26 (5):1443-1451). EGFR dimerization elicits downstream activation and signaling by several other proteins that associate with the phosphorylated tyrosines through their own phosphotyrosine-binding SH2 domains. These downstream signaling proteins initiate several signal transduction cascades, principally the MAPK, Akt and INK pathways, leading to DNA synthesis and cell proliferation (Oda K, Matsuoka Y, Funahashi A, Kitano H (2005), “A comprehensive pathway map of epidermal growth factor receptor signaling”. Mol. Syst. Biol. 1 (1): 2005.0010). Such proteins modulate phenotypes such as cell migration, adhesion, and proliferation.

Mutations that lead to EGFR overexpression (known as upregulation) or overactivity have been associated with a number of cancers, including lung cancer, anal cancers (Walker F, Abramowitz L, Benabderrahrnane D, Duval X, Descatoire V, Herrin D, Lehy T, Aparicio T (November 2009), “Growth factor receptor expression in anal squamous lesions: modifications associated with oncogenic human papillomavirus and human immunodeficiency virus”, Hum. Pathol. 40 (11): 1517-27) and glioblastoma multiforme. In this latter case, a more or less specific mutation of EGFR, called EGFRvIII is often observed (Kuan C T, Wikstrand C J, Bigner D D (June 2001), “EGF mutant receptor vIII as a molecular target in cancer therapy”, Endocr. Relat. Cancer 8 (2): 83-96). Mutations, amplifications or misregulations of EGFR or family members are implicated in about 30% of all epithelial cancers. Mutations involving EGFR could lead to its constant activation, which could result in uncontrolled cell division. Consequently, mutations of EGFR have been identified in several types of cancer, and it is the target of an expanding class of anticancer therapies (Zhang H, Berezov A, Wang Q, Zhang G, Drebin J, Murali R, Greene M I (August 2007), “ErbB receptors: from oncogenes to targeted cancer therapies”, J. Clin. Invest. 117 (8): 2051-8).

The identification of EGFR as an oncogene has led to the development of anticancer therapeutics directed against EGFR, including gefitinib and erlotinib for lung cancer, and cetuximab for colon cancer. Cetuximab and panitumumab are examples of monoclonal antibody inhibitors. Other monoclonals in clinical development are zalutumumab, nimotuzumab, and matuzumab. Another method is using small molecules to inhibit the EGFR tyrosine kinase, which is on the cytoplasmic side of the receptor. Without kinase activity, EGFR is unable to activate itself, which is a prerequisite for binding of downstream adaptor proteins. Ostensibly by halting the signaling cascade in cells that rely on this pathway for growth, tumor proliferation and migration is diminished. Gefitinib, erlotinib, and lapatinib (mixed EGFR and ERBB2 inhibitor) are examples of small molecule kinase inhibitors.

The membrane-anchored metalloproteinase TNFα convertase, TACE (also referred to as “ADAM17”) regulates the release of TNFα and EGFR-ligands from cells. As such, inhibiting TACE activity is another pathway by which EGFR activation can be blocked and represents a means of treating EGFR-dependent pathologies.

It has been found that iRhom1 and the related iRhom2 together support TACE (also referred to as ADAM17) maturation and shedding of the EGFR ligand TGFα (U.S. Pat. No. 10,024,844; and Li X et al. (May 2015), “iRhoms 1 and 2 are essential upstream regulators of ADAM17-dependent EGFR signaling”, PNAS 112(19): 6080-6085).

The iRhom2/ADAM17 complex has an essential role in the regulation of several translationally relevant signaling pathways, including the TNFα pathways (targets of anti-TNF biologics such as Etanercept or Humira), the IL-6 pathway (target of inhibitors such as Tocilizumab) and the EGFR pathways (target of inhibitors such as Erbitux). Inhibitors of iRhom2/ADAM17 would have the advantage that they target these three disease-causing pathways simultaneously. In addition, iRhom2/ADAM17 inhibitors would selectively target the more pathogenic aspects of these pathways. Specifically, the EGFR pathway has both protective function in the skin and intestinal barrier, and pathogenic functions in cancer and autoimmune diseases such as Rheumatoid Arthritis. The recent discovery that HB-EGF macrophages have an important role in RA further highlights the potential of iRhom2/ADAM17 inhibitors, which would block the pathogenic HB-EGF, without interfering with the EGFR-ligand TGFα and its role in protecting the skin and intestinal barrier (Kuo D, et al., “HBEGF+ macrophages in rheumatoid arthritis induce fibroblast invasiveness”, Sci. Transl. Med., 2019 May 8; 11(491): doi:10.1126/scitranslmed.aau8587; and Maretzky T et al. (July 2013), “iRhom2 controls the substrate selectivity of stimulated ADAM17-dependent ectodomain shedding”, PNAS 110(28): 11433-11438).

To date, there have been no small molecule inhibitors of iRhom2/ADAM17 activity disclosed to our knowledge. Thus, it remains a clinical need to discover inhibitors of iRhom2/ADAM17 activity having novel activity profiles. This application is directed to this need and others.

### SUMMARY

Provided herein are compounds that are inhibitors of iRhom2/ADAM17 activity that are useful in the treatment of various diseases related to inhibition of iRhom2/ADAM17 function or iRhom2/ADAM17 activity.

Some embodiments provide a method of inhibiting iRhom2/ADAM17 activity, said method comprising administering to a patient a compound of Formula (I):

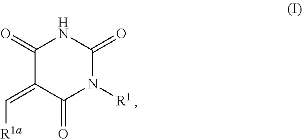

or a pharmaceutically acceptable salt thereof, wherein:
R¹ is C₆₋₁₀ aryl, 5-10 membered heteroaryl, 4-10 membered heterocycloalkyl, or C₃₋₁₀cycloalkyl;
R^{1a} is C₆₋₁₀ aryl, 5-10 membered heteroaryl, 4-10 membered heterocycloalkyl, or C₃₋₁₀ cycloalkyl;
wherein each R¹ or R^{1a} is optionally substituted with 1, 2 or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

Some embodiments provide a method of inhibiting iRhom2/ADAM17 activity, said method comprising administering to a patient a compound of Formula (II):

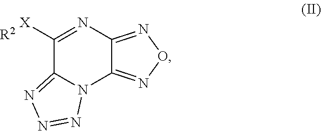

or a pharmaceutically acceptable salt thereof; wherein:
X is NH, N(C₁₋₄alkyl), O, S, S(O), S(O)₂, C(O), CH₂, CH(halo), C(halo)₂, CH(C₁₋₄alkyl), or C(C₁₋₄alkyl)₂;
R² is C₆₋₁₀ aryl, 5-10 membered heteroaryl, 4-10 membered heterocycloalkyl, or C₃₋₁₀ cycloalkyl;
wherein each R² is optionally substituted with 1, 2 or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

Some embodiments provide a method of inhibiting iRhom2/ADAM17 activity, said method comprising administering to a patient a compound of Formula (III):

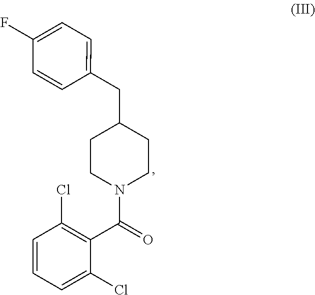 [SMILES: O=C(c1c(Cl)cccc1Cl)N1CCC(Cc2ccc(F)cc2)CC1]

or a pharmaceutically acceptable salt thereof.

Some embodiments provide a method of inhibiting iRhom2/ADAM17 activity, said method comprising administering to a patient a compound of Formula (IV):

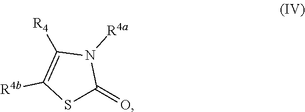

or a pharmaceutically acceptable salt thereof; wherein:
R⁴ is H, C₁₋₄ haloalkyl, or C₁₋₄ alkyl;
R^{4a} is —C₀₋₆ alkyl(C₆₋₁₀ aryl), —C₀₋₆ alkyl(5-10 membered heteroaryl), —C₀₋₆ alkyl(C₃₋₁₀cycloalkyl), or —C₀₋₆ alkyl(4-10 membered heterocycloalkyl);
R^{4b} is —C(O)—C₀₋₄alkyl(C₆₋₁₀ aryl), —C(O)—C₀₋₄alkyl(5-10 membered heteroaryl), —C(O)—C₀₋₄alkyl(C₃₋₁₀ cycloalkyl), —C(O)—C₀₋₄alkyl(4-10 membered heterocycloalkyl), —NHC(O)—C₀₋₄alkyl(C₆₋₁₀ aryl), —NHC(O)—C₀₋₄alkyl(5-10 membered heteroaryl), —NHC(O)—C₀₋₄alkyl(C₃₋₁₀ cycloalkyl), —NHC(O)—C₀₋₄alkyl(4-10 membered heterocycloalkyl), —C(O)OC₀₋₄alkyl(C₆₋₁₀ aryl), —C(O)OC₀₋₄alkyl(5-10 membered heteroaryl), —C(O)OC₀₋₄alkyl(C₃₋₁₀ cycloalkyl), —C(O)OC₀₋₄alkyl(4-10 membered heterocycloalkyl), —C(O)NH—C₀₋₄alkyl(C₆₋₁₀ aryl), —C(O)NH—C₀₋₄alkyl(5-10 membered heteroaryl), —C(O)NH—C₀₋₄alkyl(C₃₋₁₀ cycloalkyl), —C(O)NH—C₀₋₄alkyl(4-10 membered heterocycloalkyl), —C(O)N(C₁₋₆ alkyl)(C₀₋₄alkyl-C₆₋₁₀ aryl), —C(O)N(C₁₋₆ alkyl)(C₀₋₄alkyl-(5-10 membered heteroaryl)), —C(O)N(C₁₋₆ alkyl)(C₀₋₄alkyl-C₃₋₁₀ cycloalkyl), or —C(O)N(C₁₋₆ alkyl)(C₀₋₄alkyl-(4-10 membered heterocycloalkyl));
wherein each R^{4a} or R^{4b} is optionally substituted with 1, 2, or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

Some embodiments provide a method of treating a disease or disorder associated with inhibition of iRhom2/ADAM17 activity, said method comprising administering to a patient in need thereof a therapeutically effective amount of a compound of Formula (I):

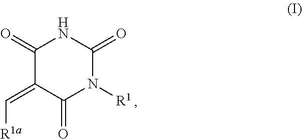

or a pharmaceutically acceptable salt thereof; wherein:
R¹ is C₆₋₁₀ aryl, 5-10 membered heteroaryl, 4-10 membered heterocycloalkyl, or C₃₋₁₀ cycloalkyl;
R^{1a} is C₆₋₁₀ aryl, 5-10 membered heteroaryl, 4-10 membered heterocycloalkyl, or C₃₋₁₀ cycloalkyl;
wherein each R¹ or R^{1a} is optionally substituted with 1, 2 or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

Some embodiments provide a method of treating a disease or disorder associated with inhibition of iRhom2/ADAM17 activity, said method comprising administering to a patient in need thereof a therapeutically effective amount of a compound of Formula (II):

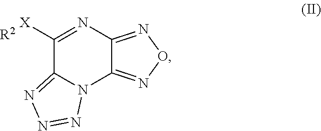

or a pharmaceutically acceptable salt thereof; wherein:
X is NH, N(C₁₋₄alkyl), O, S, S(O), S(O)₂, C(O), CH₂, CH(halo), C(halo)₂, CH(C₁₋₄alkyl), or C(C₁₋₄alkyl)₂;
R² is C₆₋₁₀ aryl, 5-10 membered heteroaryl, 4-10 membered heterocycloalkyl, or C₃₋₁₀ cycloalkyl;
wherein each R² is optionally substituted with 1, 2 or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

Some embodiments provide a method of treating a disease or disorder associated with inhibition of iRhom2/ADAM17 activity, said method comprising administering to a patient in need thereof a therapeutically effective amount of a compound of Formula (III):

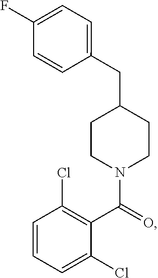 [SMILES: O=C(c1c(Cl)cccc1Cl)N1CCC(Cc2ccc(F)cc2)CC1]

or a pharmaceutically acceptable salt thereof.

Some embodiments provide a method of treating a disease or disorder associated with inhibition of iRhom2/ADAM17 activity, said method comprising administering to a patient in need thereof a therapeutically effective amount of a compound of Formula (IV):

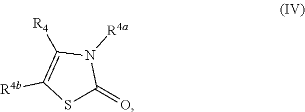

or a pharmaceutically acceptable salt thereof; wherein:
R⁴ is H, C₁₋₄ haloalkyl, or C₁₋₄ alkyl;
R^{4a} is —C₀₋₆ alkyl(C₆₋₁₀ aryl), —C₀₋₆ alkyl(5-10 membered heteroaryl), —C₀₋₆ alkyl(C₃₋₁₀cycloalkyl), or —C₀₋₆ alkyl(4-10 membered heterocycloalkyl);
R^{4b} is —C(O)—C₀₋₄alkyl(C₆₋₁₀ aryl), —C(O)—C₀₋₄alkyl(5-10 membered heteroaryl), —C(O)—C₀₋₄alkyl(C₃₋₁₀ cycloalkyl), —C(O)—C₀₋₄alkyl(4-10 membered heterocycloalkyl), —NHC(O)—C₀₋₄alkyl(C₆₋₁₀ aryl), —NHC(O)—C₀₋₄alkyl(5-10 membered heteroaryl), —NHC(O)—C₀₋₄alkyl(C₃₋₁₀ cycloalkyl), —NHC(O)—C₀₋₄alkyl(4-10 membered heterocycloalkyl), —C(O)OC₀₋₄alkyl(C₆₋₁₀ aryl), —C(O)OC₀₋₄alkyl(5-10 membered heteroaryl), —C(O)OC₀₋₄alkyl(C₃₋₁₀ cycloalkyl), —C(O)OC₀₋₄alkyl(4-10 membered heterocycloalkyl), —C(O)NH—C₀₋₄alkyl(C₆₋₁₀ aryl), —C(O)NH—C₀₋₄alkyl(5-10 membered heteroaryl), —C(O)NH—C₀₋₄alkyl(C₃₋₁₀ cycloalkyl), —C(O)NH—C₀₋₄alkyl(4-10 membered heterocycloalkyl), —C(O)N(C₁₋₆ alkyl)(C₀₋₄alkyl-C₆₋₁₀ aryl), —C(O)N(C₁₋₆ alkyl)(C₀₋₄alkyl-(5-10 membered heteroaryl)), —C(O)N(C₁₋₆ alkyl)(C₀₋₄alkyl-C₃₋₁₀ cycloalkyl), or —C(O)N(C₁₋₆ alkyl)(C₀₋₄alkyl-(4-10 membered heterocycloalkyl));
wherein each R^{4a} or R^{4b} is optionally substituted with 1, 2, or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

In some embodiments, the compound is administered to the patient in a pharmaceutical composition comprising the compound and a pharmaceutically acceptable carrier or excipient.

In some embodiments, the compound is administered to the patient in combination with one or more additional therapeutic agents.

### BRIEF DESCRIPTION OF THE FIGURES

FIG. 1 shows the sequence for KL2-AP (SEQ ID NO:1) and the result of translation (SEQ ID NO:2) as noted in Example 2.

FIG. 2 shows the sequence for TGFα-AP (SEQ ID NO:3) and the result of translation (SEQ ID NO:4) as noted in Example 2.

FIG. 3a shows results from TNFα release assays, demonstrating that Compound 1 and Compound 2 interfere with LPS-induced shedding of TNFα in human THP-1 macrophage cells. The data illustrate the effects of test articles in absolute numbers of released TNFα.

FIG. 3b shows results from TNFα release assays, demonstrating that Compounds 3, 4, 5, and 7 interfere with LPS-induced shedding of TNFα in human THP-1 macrophage cells. The data illustrate the effects of test articles in absolute numbers of released TNFα.

FIG. 4a shows results from TNFα release assays, demonstrating that Compound 1 and Compound 2 interfere with LPS-induced shedding of TNFα in human THP-1 macrophage cells. The data illustrate the effects of test articles on TNFα release in percent inhibition.

FIG. 4b shows results from TNFα release assays, demonstrating that Compounds 3, 4, 5, and 7 interfere with LPS-induced shedding of TNFα in human THP-1 macrophage cells. The data illustrate the effects of test articles on TNFα release in percent inhibition.

FIG. 5a shows results from TNFα release assays, demonstrating that Compound 1 and Compound 2 interfere with LPS-induced shedding of TNFα in human peripheral blood mononuclear cells (PBMCs) isolated from healthy donor. The data illustrate the effects of test articles in absolute numbers of released TNFα.

FIG. 5b shows results from TNFα release assays, demonstrating that Compounds 3, 4, 5, and 7 interfere with LPS-induced shedding of TNFα in human peripheral blood mononuclear cells (PBMCs) isolated from healthy donor. The data illustrate the effects of test articles in absolute numbers of released TNFα.

FIG. 6a shows results from TNFα release assays, demonstrating that Compound 1 and Compound 2 interfere with LPS-induced shedding of TNFα in human peripheral blood mononuclear cells (PBMCs) isolated from healthy donor. The data illustrate the effects of test articles on TNFα release in percent inhibition.

FIG. 6b shows results from TNFα release assays, demonstrating that Compounds 3, 4, 5, and 7 interfere with LPS-induced shedding of TNFα in human peripheral blood mononuclear cells (PBMCs) isolated from healthy donor. The data illustrate the effects of test articles on TNFα release in percent inhibition.

FIG. 7a shows results from IL-6R release assays, demonstrating that Compound 1 and Compound 2 interfere with PMA-induced shedding of IL-6R in human peripheral blood mononuclear cells (PBMCs) isolated from healthy donor. The data illustrate the effects of test articles in absolute numbers of released IL-6R.

FIG. 7b shows results from IL-6R release assays, demonstrating that Compounds 4, 5, and 7 interfere with PMA-induced shedding of IL-6R in human peripheral blood mononuclear cells (PBMCs) isolated from healthy donor. The data illustrate the effects of test articles in absolute numbers of released IL-6R.

FIG. 8a shows results from IL-6R release assays, demonstrating that Compound 1 and Compound 2 interfere with PMA-induced shedding of IL-6R in human peripheral blood mononuclear cells (PBMCs) isolated from healthy donor. The data illustrate the effects of test articles on IL-6R release in percent inhibition.

FIG. 8b shows results from IL-6R release assays, demonstrating that Compounds 4, 5, and 7 interfere with PMA-induced shedding of IL-6R in human peripheral blood mononuclear cells (PBMCs) isolated from healthy donor. The data illustrate the effects of test articles on IL-6R release in percent inhibition.

FIG. 9a shows results from HB-EGF release assays, demonstrating that Compound 1 and Compound 2 interfere with PMA-induced shedding of HB-EGF in THP1 cells. The data illustrate the effects of test articles in absolute numbers of released HB-EGF.

FIG. 9b shows results from HB-EGF release assays, demonstrating that Compounds 3, 4, 5, and 7 interfere with PMA-induced shedding of HB-EGF in THP1 cells. The data illustrate the effects of test articles in absolute numbers of released HB-EGF.

FIG. 10a shows results from HB-EGF release assays, demonstrating that Compound 1 and Compound 2 interfere with PMA-induced shedding of HB-EGF in THP1 cells. The data illustrate the effects of test articles on HB-EGF release in percent inhibition.

FIG. 10b shows results from HB-EGF release assays, demonstrating that Compounds 3, 4, 5, and 7 interfere with PMA-induced shedding of HB-EGF in THP1 cells. The data illustrate the effects of test articles on HB-EGF release in percent inhibition.

FIG. 11a shows results from HB-EGF release assays, demonstrating that Compound 1 and Compound 2 interfere with PMA-induced shedding of HB-EGF in human peripheral blood mononuclear cells (PBMCs) isolated from healthy donor. The data illustrate the effects of test articles in absolute numbers of released HB-EGF.

FIG. 11b shows results from HB-EGF release assays, demonstrating that Compounds 3, 4, 5, and 7 interfere with PMA-induced shedding of HB-EGF in human peripheral blood mononuclear cells (PBMCs) isolated from healthy donor. The data illustrate the effects of test articles in absolute numbers of released HB-EGF.

FIG. 12a shows results from HB-EGF release assays, demonstrating that Compound 1 and Compound 2 interfere with PMA-induced shedding of HB-EGF in human peripheral blood mononuclear cells (PBMCs) isolated from healthy donor. The data illustrate the effects of test articles on HB-EGF release in percent inhibition.

FIG. 12b shows results from HB-EGF release assays, demonstrating that Compounds 3, 4, 5, and 7 interfere with PMA-induced shedding of HB-EGF in human peripheral blood mononuclear cells (PBMCs) isolated from healthy donor. The data illustrate the effects of test articles on HB-EGF release in percent inhibition.

FIG. 13a shows results from TGFα release assays, demonstrating that Compound 1 and Compound 2 only weakly interfere with PMA-induced shedding of TGFα in human PC3 prostate cancer cells. The data illustrate the effects of test articles in absolute numbers of released TGFα.

FIG. 13b shows results from TGFα release assays, demonstrating that Compounds 3, 4, 5, and 7 only weakly interfere with PMA-induced shedding of TGFα in human PC3 prostate cancer cells. The data illustrate the effects of test articles in absolute numbers of released TGFα.

FIG. 14a shows results from TGFα release assays, demonstrating that Compound 1 and Compound 2 only weakly interfere with PMA-induced shedding of TGFα in human PC3 prostate cancer cells. The data illustrate the effects of test articles on TGFα release in percent inhibition.

FIG. 14b shows results from TGFα release assays, demonstrating that Compounds 3, 4, 5, and 7 only weakly interfere with PMA-induced shedding of TGFα in human PC3 prostate cancer cells. The data illustrate the effects of test articles on TGFα release in percent inhibition.

### DETAILED DESCRIPTION

In some embodiments, the present disclosure provides, inter alia, a method of inhibiting iRhom2/ADAM17 activity, said method comprising administering to a patient a compound of the disclosure, or a pharmaceutically acceptable salt thereof.

In some embodiments, the present disclosure provides, inter alia, a method of treating a disease or disorder associated with inhibition of iRhom2/ADAM17 activity, said method comprising administering to a patient in need thereof a therapeutically effective amount of a compound of the disclosure, or a pharmaceutically acceptable salt thereof.

### I. Compounds of the Disclosure

Presented herein is a compound of Formula (I):

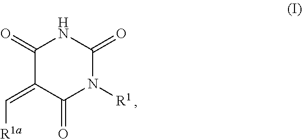

or a pharmaceutically acceptable salt thereof; wherein:
R¹ is C₆₋₁₀ aryl, 5-10 membered heteroaryl, 4-10 membered heterocycloalkyl, or C₃₋₁₀ cycloalkyl;
R^{1a} is C₆₋₁₀ aryl, 5-10 membered heteroaryl, 4-10 membered heterocycloalkyl, or C₃₋₁₀cycloalkyl;
wherein each R¹ or R^{1a} is optionally substituted with 1, 2 or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

In some embodiments, R¹ is C₆₋₁₀ aryl optionally substituted with 1, 2 or 3 substituents selected from the group consisting of halo and C₁₋₆ alkyl.

In some embodiments, R¹ is

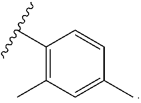 [SMILES: *c1ccc(C)cc1C]

In some embodiments, R^{1a} is a 5-10 membered heteroaryl optionally substituted with 1, 2 or 3 substituents selected from the group consisting of halo, C₁₋₆ alkyl, C₁₋₄ alkyl(NHC(O)C₁₋₄ alkyl), C₁₋₄ alkyl(C(O)C₁₋₄ alkyl), C₁₋₄ alkyl(C(O)NHC₁₋₄ alkyl), C₁₋₄ alkyl(OC(O)C₁₋₄ alkyl) and C₁₋₄ alkyl((O)COC₁₋₄ alkyl),

In some embodiments, R^{1a} is indole optionally substituted with 1, 2 or 3 substituents selected from the group consisting of halo, C₁₋₆ alkyl, C₁₋₄ alkyl(NHC(O)C₁₋₄ alkyl), C₁₋₄ alkyl(C(O)C₁₋₄ alkyl), C₁₋₄ alkyl(C(O)NHC₁₋₄ alkyl), C₁₋₄ alkyl(OC(O)C₁₋₄ alkyl) and C₁₋₄ alkyl((O)COC₁₋₄ alkyl).

In some embodiments, R^{1a} is

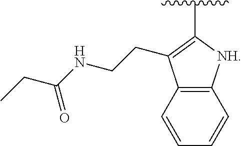 [SMILES: *c1[nH]c2ccccc2c1CCNC(=O)CC]

In some embodiments, the compound of Formula (I) is:

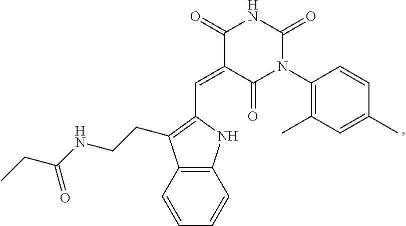 [SMILES: CCC(=O)NCCc1c(/C=C2/C(=O)NC(=O)N(c3ccc(C)cc3C)C2=O)[nH]c2ccccc12]

or a pharmaceutically acceptable salt thereof.

Presented herein is a compound of Formula (II):

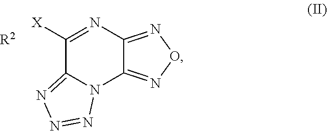

or a pharmaceutically acceptable salt thereof; wherein:
X is NH, N(C₁₋₄alkyl), O, S, S(O), S(O)₂, C(O), CH₂, CH(halo), C(halo)₂, CH(C₁₋₄alkyl), or C(C₁₋₄alkyl)₂;
R² is C₆₋₁₀ aryl, 5-10 membered heteroaryl, 4-10 membered heterocycloalkyl, or C₃₋₁₀ cycloalkyl;
wherein each R² is optionally substituted with 1, 2 or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

In some embodiments, X is NH.

In some embodiments, R² is C₆₋₁₀ aryl optionally substituted with 1, 2 or 3 substituents selected from the group consisting of halo, C₁₋₆ alkyl, —C(O)(C₁₋₄ alkyl), —C(O)NH₂, —C(O)NH(C₁₋₄ alkyl), —C(O)N(C₁₋₄ alkyl)₂, —C(O)O(C₁₋₄ alkyl), S(O)(C₁₋₄ alkyl), —S(O)NH₂, —S(O)NH(C₁₋₄ alkyl), —S(O)N(C₁₋₄ alkyl)₂, —S(O)₂(C₁₋₄ alkyl), —S(O)₂NH₂, —S(O)₂NH(C₁₋₄ alkyl), and —S(O)₂N(C₁₋₄ alkyl)₂.

In some embodiments, R² is

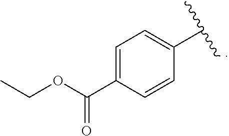 [SMILES: *c1ccc(C(=O)OCC)cc1]

In some embodiments, the compound of Formula (II) is:

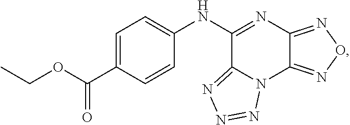 [SMILES: CCOC(=O)c1ccc(Nc2nc3nonc3n3nnnc23)cc1]

or a pharmaceutically acceptable salt thereof.

Presented herein is a compound of Formula (III):

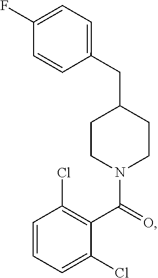 [SMILES: O=C(c1c(Cl)cccc1Cl)N1CCC(Cc2ccc(F)cc2)CC1]

or a pharmaceutically acceptable salt thereof.

Presented herein is a compound of Formula (IV):

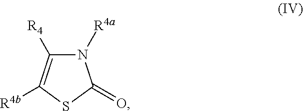

or a pharmaceutically acceptable salt thereof; wherein:
R⁴ is H, C₁₋₄ haloalkyl, or C₁₋₄ alkyl;
R^{4a} is —C₀₋₆ alkyl(C₆₋₁₀ aryl), —C₀₋₆ alkyl(5-10 membered heteroaryl), —C₀₋₆ alkyl(C₃₋₁₀ cycloalkyl), or —C₀₋₆ alkyl(4-10 membered heterocycloalkyl);
R^{4b} is —C(O)—C₀₋₄alkyl(C₆₋₁₀ aryl), —C(O)—C₀₋₄alkyl(5-10 membered heteroaryl), —C(O)—C₀₋₄alkyl(C₃₋₁₀cycloalkyl), —C(O)—C₀₋₄alkyl(4-10 membered heterocycloalkyl), —NHC(O)—C₀₋₄alkyl(C₆₋₁₀ aryl), —NHC(O)—C₀₋₄alkyl(5-10 membered heteroaryl), —NHC(O)—C₀₋₄alkyl(C₃₋₁₀ cycloalkyl), —NHC(O)—C₀₋₄alkyl(4-10 membered heterocycloalkyl), —C(O)OC₀₋₄alkyl(C₆₋₁₀ aryl), —C(O)OC₀₋₄alkyl(5-10 membered heteroaryl), —C(O)OC₀₋₄alkyl(C₃₋₁₀ cycloalkyl), —C(O)OC₀₋₄alkyl(4-10 membered heterocycloalkyl), —C(O)NH—C₀₋₄alkyl(C₆₋₁₀ aryl), —C(O)NH—C₀₋₄alkyl(5-10 membered heteroaryl), —C(O)NH—C₀₋₄alkyl(C₃₋₁₀cycloalkyl), —C(O)NH—C₀₋₄alkyl(4-10 membered heterocycloalkyl), —C(O)N(C₁₋₆ alkyl)(C₀₋₄alkyl-C₆₋₁₀ aryl), —C(O)N(C₁₋₆ alkyl)(C₀₋₄alkyl-(5-10 membered heteroaryl)), —C(O)N(C₁₋₆ alkyl)(C₀₋₄alkyl-C₃₋₁₀ cycloalkyl), or —C(O)N(C₁₋₆ alkyl)(C₀₋₄alkyl-(4-10 membered heterocycloalkyl));
wherein each R^{4a} or R^{4b} is optionally substituted with 1, 2, or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀cycloalkyl), and —C₀₋₄ alkyl(NHC(O)-(4-10 membered heterocycloalkyl)).

In some embodiments, R⁴ is methyl.

In some embodiments, R^{4a} is —C₁₋₆ alkyl(C₆₋₁₀ aryl) optionally substituted with 1, 2, or 3 substituents selected from the group consisting of halo and —OC(O)(C₁₋₄ alkyl).

In some embodiments, R^{4a} is selected from the group consisting of

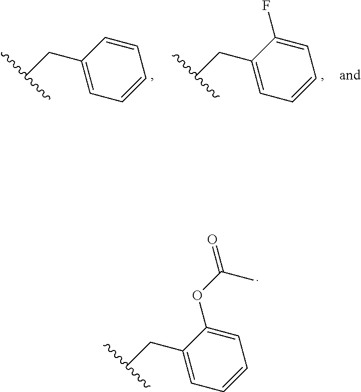 [SMILES: *Cc1ccccc1 ; *Cc1ccccc1F ; *Cc1ccccc1OC(C)=O]

In some embodiments, R^{4b} is —C(O)NH—C₁₋₄alkyl(C₆₋₁₀ aryl), —C(O)NH—C₁₋₄alkyl(5-10 membered heteroaryl), —C(O)NH—C₁₋₄alkyl(C₃₋₁₀ cycloalkyl), —C(O)NH—C₁₋₄alkyl(4-10 membered heterocycloalkyl), —C(O)N(C₁₋₆ alkyl)(C₁₋₄alkyl-C₆₋₁₀ aryl), —C(O)N(C₁₋₆ alkyl)(C₁₋₄alkyl-(5-10 membered heteroaryl)), —C(O)N(C₁₋₆ alkyl)(C₁₋₄alkyl-C₃₋₁₀cycloalkyl), or —C(O)N(C₁₋₆ alkyl)(C₁₋₄alkyl-(4-10 membered heterocycloalkyl)).

In some embodiments, R^{4b} is —C(O)NH—C₁₋₄alkyl(5-10 membered heteroaryl).

In some embodiments, R^{4b} is selected from the group consisting of

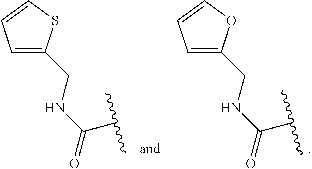 [SMILES: *C(=O)NCc1cccs1 ; *C(=O)NCc1ccco1]

In some embodiments, the compound of Formula (IV) is:

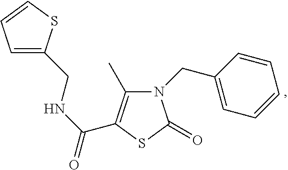 [SMILES: Cc1c(C(=O)NCc2cccs2)sc(=O)n1Cc1ccccc1]

or a pharmaceutically acceptable salt thereof.

In some embodiments, the compound of Formula (IV) is:

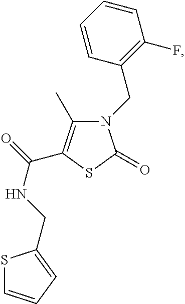 [SMILES: Cc1c(C(=O)NCc2cccs2)sc(=O)n1Cc1ccccc1F]

or a pharmaceutically acceptable salt thereof.

In some embodiments, the compound of Formula (IV) is:

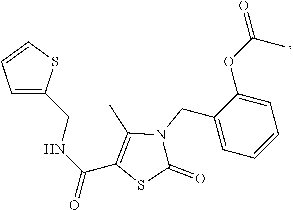 [SMILES: CC(=O)Oc1ccccc1Cn1c(C)c(C(=O)NCc2cccs2)sc1=O]

or a pharmaceutically acceptable salt thereof.

In some embodiments, the compound of Formula (IV) is:

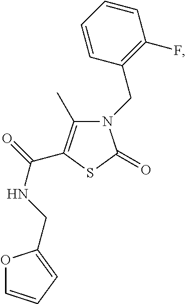 [SMILES: Cc1c(C(=O)NCc2ccco2)sc(=O)n1Cc1ccccc1F]

or a pharmaceutically acceptable salt thereof.

Specific compounds used in the methods of the present disclosure are listed in Table 1 below. The compounds in Table 1 were obtained either from commercial sources through Evotec (Hamburg, Germany), i.e., AKos Consulting & Solutions GmbH (Lörrach, Germany) and MolPort (Beacon, NY, USA); or from commercial sources directly, i.e., Ambinter (Orleans, France); and AKos Consulting & Solutions GmbH (Lörrach, Germany).

The Evotec ID number and/or representative commercial source with commercial ID number for each compound are noted in Table 1.

TABLE 1

| | |
|---|---|
| 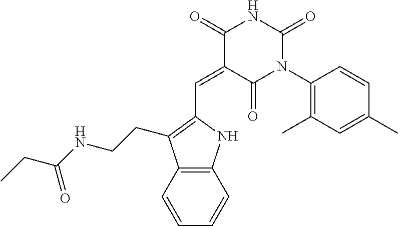 [SMILES: CCC(=O)NCCc1c(/C=C2/C(=O)NC(=O)N(c3ccc(C)cc3C)C2=O)[nH]c2ccccc12] | 1 |
| MolPort; MolPort-002-519-968 | |
| 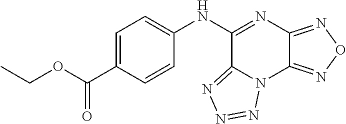 [SMILES: CCOC(=O)c1ccc(Nc2nc3nonc3n3nnnc23)cc1] | 2 |
| AKos: AKOS024356657 | |
| 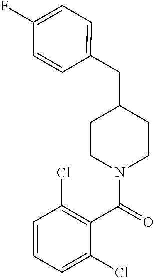 [SMILES: O=C(c1c(Cl)cccc1Cl)N1CCC(Cc2ccc(F)cc2)CC1] | 3 |
| AKos; #SR_103 | |
| 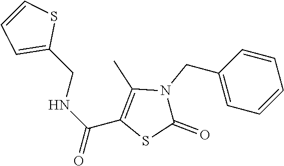 [SMILES: Cc1c(C(=O)NCc2cccs2)sc(=O)n1Cc1ccccc1] | 4 |
| AKos; #SR_104 | |
| 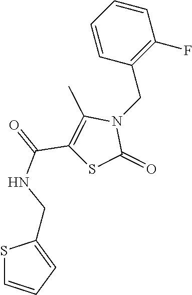 [SMILES: Cc1c(C(=O)NCc2cccs2)sc(=O)n1Cc1ccccc1F] | 5 |
| AKos; #SR_105 | |
| 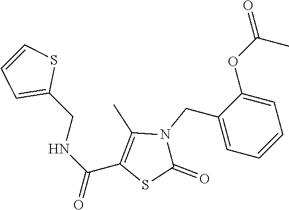 [SMILES: CC(=O)Oc1ccccc1Cn1c(C)c(C(=O)NCc2cccs2)sc1=O] | 6 |
| Ambinter; #Amb45730695 | |
| 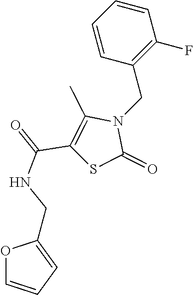 [SMILES: Cc1c(C(=O)NCc2ccco2)sc(=O)n1Cc1ccccc1F] | 7 |
| AKos; #SR_107 | |

It is further appreciated that certain features of the disclosure, which are, for clarity, described in the context of separate embodiments, can also be provided in combination in a single embodiment (while the embodiments are intended to be combined as if written in multiply dependent form). Conversely, various features of the disclosure which are, for brevity, described in the context of a single embodiment, can also be provided separately or in any suitable subcombination. Thus, it is contemplated as features described as embodiments of the compounds of the disclosure can be combined in any suitable combination.

At various places in the present specification, certain features of the compounds are disclosed in groups or in ranges. It is specifically intended that such a disclosure include each and every individual subcombination of the members of such groups and ranges. For example, the term “C₁₋₆ alkyl” is specifically intended to individually disclose (without limitation) methyl, ethyl, C₃ alkyl, C₄ alkyl, C₅ alkyl and C₆ alkyl.

The term “n-membered,” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring and 1,2,3,4-tetrahydro-naphthalene is an example of a 10-membered cycloalkyl group.

At various places in the present specification, variables defining divalent linking groups may be described. It is specifically intended that each linking substituent include both the forward and backward forms of the linking substituent. For example, —NR(CR′R″)ₙ-includes both —NR(CR′R″)ₙ— and —(CR′R″)ₙNR— and is intended to disclose each of the forms individually. Where the structure requires a linking group, the Markush variables listed for that group are understood to be linking groups. For example, if the structure requires a linking group and the Markush group definition for that variable lists “alkyl” or “aryl” then it is understood that the “alkyl” or “aryl” represents a linking alkylene group or arylene group, respectively.

The term “substituted” means that an atom or group of atoms formally replaces hydrogen as a “substituent” attached to another group. The term “substituted”, unless otherwise indicated, refers to any level of substitution, e.g., mono-, di-, tri-, tetra- or penta-substitution, where such substitution is permitted. The substituents are independently selected, and substitution may be at any chemically accessible position. It is to be understood that substitution at a given atom is limited by valency. It is to be understood that substitution at a given atom results in a chemically stable molecule. The phrase “optionally substituted” means unsubstituted or substituted. The term “substituted” means that a hydrogen atom is removed and replaced by a substituent. A single divalent substituent, e.g., oxo, can replace two hydrogen atoms.

The term “Cₙ₋ₘ” indicates a range which includes the endpoints, wherein n and m are integers and indicate the number of carbons. Examples include C₁₋₄, C₁₋₆ and the like.

The term “alkyl” employed alone or in combination with other terms, refers to a saturated hydrocarbon group that may be straight-chained or branched. The term “Cₙ₋ₘ alkyl”, refers to an alkyl group having n to m carbon atoms. An alkyl group formally corresponds to an alkane with one C—H bond replaced by the point of attachment of the alkyl group to the remainder of the compound. In some embodiments, the alkyl group contains from 1 to 6 carbon atoms, from 1 to 4 carbon atoms, from 1 to 3 carbon atoms, or 1 to 2 carbon atoms. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl and the like.

The term “alkenyl” employed alone or in combination with other terms, refers to a straight-chain or branched hydrocarbon group corresponding to an alkyl group having one or more double carbon-carbon bonds. An alkenyl group formally corresponds to an alkene with one C—H bond replaced by the point of attachment of the alkenyl group to the remainder of the compound. The term “Cₙ₋ₘ alkenyl” refers to an alkenyl group having n to m carbons. In some embodiments, the alkenyl moiety contains 2 to 6, 2 to 4, or 2 to 3 carbon atoms. Example alkenyl groups include, but are not limited to, ethenyl, n-propenyl, isopropenyl, n-butenyl, sec-butenyl and the like.

The term “alkynyl” employed alone or in combination with other terms, refers to a straight-chain or branched hydrocarbon group corresponding to an alkyl group having one or more triple carbon-carbon bonds. An alkynyl group formally corresponds to an alkyne with one C—H bond replaced by the point of attachment of the alkyl group to the remainder of the compound. The term “Cₙ₋ₘ alkynyl” refers to an alkynyl group having n to m carbons. Example alkynyl groups include, but are not limited to, ethynyl, propyn-1-yl, propyn-2-yl and the like. In some embodiments, the alkynyl moiety contains 2 to 6, 2 to 4, or 2 to 3 carbon atoms.

The term “alkylene”, employed alone or in combination with other terms, refers to a divalent alkyl linking group. An alkylene group formally corresponds to an alkane with two C—H bond replaced by points of attachment of the alkylene group to the remainder of the compound. The term “Cₙ₋ₘ alkylene” refers to an alkylene group having n to m carbon atoms. Examples of alkylene groups include, but are not limited to, ethan-1,2-diyl, propan-1,3-diyl, propan-1,2-diyl, butan-1,4-diyl, butan-1,3-diyl, butan-1,2-diyl, 2-methyl-propan-1,3-diyl and the like.

The term “alkoxy”, employed alone or in combination with other terms, refers to a group of formula —O-alkyl, wherein the alkyl group is as defined above. The term “Cₙ₋ₘ alkoxy” refers to an alkoxy group, the alkyl group of which has n to m carbons. Example alkoxy groups include methoxy, ethoxy, propoxy (e.g., n-propoxy and isopropoxy), t-butoxy and the like. In some embodiments, the alkyl group has 1 to 6, 1 to 4, or 1 to 3 carbon atoms.

The term “amino” refers to a group of formula —NH₂.

The term “carbamyl” refers to a group of formula —C(O)NH₂.

The term “carbonyl”, employed alone or in combination with other terms, refers to a —C(═O)— group, which also may be written as C(O).

The term “cyano” or “nitrile” refers to a group of formula —C≡N, which also may be written as —CN.

The terms “halo” or “halogen”, used alone or in combination with other terms, refers to fluoro, chloro, bromo and iodo. In some embodiments, “halo” refers to a halogen atom selected from F, Cl, or Br. In some embodiments, halo groups are F.

The term “haloalkyl” as used herein refers to an alkyl group in which one or more of the hydrogen atoms has been replaced by a halogen atom. The term “Cₙ₋ₘ haloalkyl” refers to a Cₙ₋ₘ alkyl group having n to m carbon atoms and from at least one up to {2(n to m)+1}halogen atoms, which may either be the same or different. In some embodiments, the halogen atoms are fluoro atoms. In some embodiments, the haloalkyl group has 1 to 6 or 1 to 4 carbon atoms. Example haloalkyl groups include CF₃, C₂F₅, CHF₂, CCl₃, CHCl₂, C₂Cl₅ and the like. In some embodiments, the haloalkyl group is a fluoroalkyl group.

The term “haloalkoxy”, employed alone or in combination with other terms, refers to a group of formula —O-haloalkyl, wherein the haloalkyl group is as defined above. The term “C-m haloalkoxy” refers to a haloalkoxy group, the haloalkyl group of which has n to m carbons. Example haloalkoxy groups include trifluoromethoxy and the like. In some embodiments, the haloalkoxy group has 1 to 6, 1 to 4, or 1 to 3 carbon atoms.

The term “oxo” refers to an oxygen atom as a divalent substituent, forming a carbonyl group when attached to carbon, or attached to a heteroatom forming a sulfoxide or sulfone group, or an N-oxide group. In some embodiments, heterocyclic groups may be optionally substituted by 1 or 2 oxo (═O) substituents.

The term “aromatic” refers to a carbocycle or heterocycle having one or more polyunsaturated rings having aromatic character (i.e., having (4n+2) delocalized 7 (pi) electrons where n is an integer).

The term “aryl,” employed alone or in combination with other terms, refers to an aromatic hydrocarbon group, which may be monocyclic or polycyclic (e.g., having 2 fused rings). The term “Cₙ₋ₘ aryl” refers to an aryl group having from n to m ring carbon atoms. Aryl groups include, e.g., phenyl, naphthyl, indanyl, indenyl and the like. In some embodiments, aryl groups have from 6 to about 10 carbon atoms. In some embodiments aryl groups have 6 carbon atoms. In some embodiments aryl groups have 10 carbon atoms. In some embodiments, the aryl group is phenyl. In some embodiments, the aryl group is naphthyl.

The term “heteroatom” used herein is meant to include boron, phosphorus, sulfur, oxygen and nitrogen.

The term “heteroaryl” or “heteroaromatic,” employed alone or in combination with other terms, refers to a monocyclic or polycyclic aromatic heterocycle having at least one heteroatom ring member selected from boron, phosphorus, sulfur, oxygen and nitrogen. In some embodiments, the heteroaryl ring has 1, 2, 3 or 4 heteroatom ring members independently selected from nitrogen, sulfur and oxygen. In some embodiments, any ring-forming N in a heteroaryl moiety can be an N-oxide. In some embodiments, the heteroaryl has 5-14 ring atoms including carbon atoms and 1, 2, 3 or 4 heteroatom ring members independently selected from nitrogen, sulfur and oxygen. In some embodiments, the heteroaryl has 5-14, or 5-10 ring atoms including carbon atoms and 1, 2, 3 or 4 heteroatom ring members independently selected from nitrogen, sulfur and oxygen. In some embodiments, the heteroaryl has 5-6 ring atoms and 1 or 2 heteroatom ring members independently selected from nitrogen, sulfur and oxygen. In some embodiments, the heteroaryl is a five-membered or six-membered heteroaryl ring. In other embodiments, the heteroaryl is an eight-membered, nine-membered or ten-membered fused bicyclic heteroaryl ring. Example heteroaryl groups include, but are not limited to, pyridinyl (pyridyl), pyrimidinyl, pyrazinyl, pyridazinyl, pyrrolyl, pyrazolyl, azolyl, oxazolyl, thiazolyl, imidazolyl, furanyl, thiophenyl, quinolinyl, isoquinolinyl, naphthyridinyl (including 1,2-, 1,3-, 1,4-, 1,5-, 1,6-, 1,7-, 1,8-, 2,3- and 2,6-naphthyridine), indolyl, benzothiophenyl, benzofuranyl, benzisoxazolyl, imidazo[1,2-b]thiazolyl, purinyl, and the like.

A five-membered heteroaryl ring is a heteroaryl group having five ring atoms wherein one or more (e.g., 1, 2 or 3) ring atoms are independently selected from N, O and S. Exemplary five-membered ring heteroaryls include thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl and 1,3,4-oxadiazolyl.

A six-membered heteroaryl ring is a heteroaryl group having six ring atoms wherein one or more (e.g., 1, 2 or 3) ring atoms are independently selected from N, O and S. Exemplary six-membered ring heteroaryls are pyridyl, pyrazinyl, pyrimidinyl, triazinyl and pyridazinyl.

The term “cycloalkyl,” employed alone or in combination with other terms, refers to a non-aromatic hydrocarbon ring system (monocyclic, bicyclic or polycyclic), including cyclized alkyl and alkenyl groups. The term “Cₙ₋ₘ cycloalkyl” refers to a cycloalkyl that has n to m ring member carbon atoms. Cycloalkyl groups can include mono- or polycyclic (e.g., having 2, 3 or 4 fused rings) groups and spirocycles. Cycloalkyl groups can have 3, 4, 5, 6, 7, 8, 9, 10, 11, 12, 13, or 14 ring-forming carbons (C₃₋₁₄). In some embodiments, the cycloalkyl group has 3 to 14 members, 3 to 10 members, 3 to 6 ring members, 3 to 5 ring members, or 3 to 4 ring members. In some embodiments, the cycloalkyl group is monocyclic. In some embodiments, the cycloalkyl group is monocyclic or bicyclic. In some embodiments, the cycloalkyl group is a C₃₋₆ monocyclic cycloalkyl group. Ring-forming carbon atoms of a cycloalkyl group can be optionally oxidized to form an oxo or sulfido group. Cycloalkyl groups also include cycloalkylidenes. In some embodiments, cycloalkyl is cyclopropyl, cyclobutyl, cyclopentyl or cyclohexyl. Also included in the definition of cycloalkyl are moieties that have one or more aromatic rings fused (i.e., having a bond in common with) to the cycloalkyl ring, e.g., benzo or thienyl derivatives of cyclopentane, cyclohexane and the like. A cycloalkyl group containing a fused aromatic ring can be attached through any ring-forming atom including a ring-forming atom of the fused aromatic ring. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, bicyclo[1.1.1]pentanyl, bicyclo[2.1.1]hexanyl, and the like. In some embodiments, the cycloalkyl group is cyclopropyl, cyclobutyl, cyclopentyl, or cyclohexyl.

The term “heterocycloalkyl,” employed alone or in combination with other terms, refers to a non-aromatic ring or ring system, which may optionally contain one or more alkenylene groups as part of the ring structure, which has at least one heteroatom ring member independently selected from boron, nitrogen, sulfur oxygen and phosphorus, and which has 4-14 ring members, 4-10 ring members, 4-7 ring members, or 4-6 ring members. Included within the term “heterocycloalkyl” are monocyclic 4-, 5-, 6- and 7-membered heterocycloalkyl groups. Heterocycloalkyl groups can include mono- or bicyclic or polycyclic (e.g., having two or three fused or bridged rings) ring systems or spirocycles. In some embodiments, the heterocycloalkyl group is a monocyclic group having 1, 2 or 3 heteroatoms independently selected from nitrogen, sulfur and oxygen. Ring-forming carbon atoms and heteroatoms of a heterocycloalkyl group can be optionally oxidized to form an oxo or sulfido group or other oxidized linkage (e.g., C(O), S(O), C(S) or S(O)₂, N-oxide etc.) or a nitrogen atom can be quaternized. The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. Also included in the definition of heterocycloalkyl are moieties that have one or more aromatic rings fused (i.e., having a bond in common with) to the heterocycloalkyl ring, e.g., benzo or thienyl derivatives of piperidine, morpholine, azepine, etc. A heterocycloalkyl group containing a fused aromatic ring can be attached through any ring-forming atom including a ring-forming atom of the fused aromatic ring. Examples of heterocycloalkyl groups include azetidinyl, azepanyl, dihydrobenzofuranyl, dihydrofuranyl, dihydropyranyl, morpholino, 3-oxa-9-azaspiro[5.5]undecanyl, 1-oxa-8-azaspiro[4.5]decanyl, piperidinyl, piperazinyl, oxopiperazinyl, pyranyl, pyrrolidinyl, quinuclidinyl, tetrahydrofuranyl, tetrahydropyranyl, 1,2,3,4-tetrahydroquinolinyl, tropanyl, 4,5,6,7-tetrahydrothiazolo[5,4-c]pyridinyl, and thiomorpholino.

At certain places, the definitions or embodiments refer to specific rings (e.g., an azetidine ring, a pyridine ring, etc.). Unless otherwise indicated, these rings can be attached to any ring member provided that the valency of the atom is not exceeded. For example, an azetidine ring may be attached at any position of the ring, whereas an azetidin-3-yl ring is attached at the 3-position.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically inactive starting materials are known in the art, such as by resolution of racemic mixtures or by stereoselective synthesis. Many geometric isomers of olefins, C═N double bonds and the like can also be present in the compounds described herein, and all such stable isomers are contemplated in the present invention. Cis and trans geometric isomers of the compounds of the present invention are described and may be isolated as a mixture of isomers or as separated isomeric forms.

Resolution of racemic mixtures of compounds can be carried out by any of numerous methods known in the art. One method includes fractional recrystallization using a chiral resolving acid which is an optically active, salt-forming organic acid. Suitable resolving agents for fractional recrystallization methods are, e.g., optically active acids, such as the D and L forms of tartaric acid, diacetyltartaric acid, dibenzoyltartaric acid, mandelic acid, malic acid, lactic acid or the various optically active camphorsulfonic acids such as (3-camphorsulfonic acid. Other resolving agents suitable for fractional crystallization methods include stereoisomerically pure forms of α-methylbenzylamine (e.g., S and R forms, or diastereomerically pure forms), 2-phenylglycinol, norephedrine, ephedrine, N-methylephedrine, cyclohexylethylamine, 1,2-diaminocyclohexane and the like.

Resolution of racemic mixtures can also be carried out by elution on a column packed with an optically active resolving agent (e.g., dinitrobenzoylphenylglycine). Suitable elution solvent composition can be determined by one skilled in the art.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide—imidic acid pairs, lactam—lactim pairs, enamine—imine pairs, and annular forms where a proton can occupy two or more positions of a heterocyclic system, e.g., 1H- and 3H-imidazole, 1H-, 2H- and 4H-1,2,4-triazole, 1H- and 2H-isoindole and 1H- and 2H-pyrazole. Tautomeric forms can be in equilibrium or sterically locked into one form by appropriate substitution.

Compounds of the invention can also include all isotopes of atoms occurring in the intermediates or final compounds. Isotopes include those atoms having the same atomic number but different mass numbers. For example, isotopes of hydrogen include tritium and deuterium. One or more constituent atoms of the compounds of the invention can be replaced or substituted with isotopes of the atoms in natural or non-natural abundance. In some embodiments, the compound includes at least one deuterium atom. For example, one or more hydrogen atoms in a compound of the present disclosure can be replaced or substituted by deuterium. In some embodiments, the compound includes two or more deuterium atoms. In some embodiments, the compound includes 1, 2, 3, 4, 5, 6, 7, 8, 9, 10, 11 or 12 deuterium atoms. Synthetic methods for including isotopes into organic compounds are known in the art.

The term, “compound,” as used herein is meant to include all stereoisomers, geometric isomers, tautomers and isotopes of the structures depicted. The term is also meant to refer to compounds of the inventions, regardless of how they are prepared, e.g., synthetically, through biological process (e.g., metabolism or enzyme conversion), or a combination thereof.

All compounds, and pharmaceutically acceptable salts thereof, can be found together with other substances such as water and solvents (e.g., hydrates and solvates) or can be isolated. When in the solid state, the compounds described herein and salts thereof may occur in various forms and may, e.g., take the form of solvates, including hydrates. The compounds may be in any solid state form, such as a polymorph or solvate, so unless clearly indicated otherwise, reference in the specification to compounds and salts thereof should be understood as encompassing any solid state form of the compound.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The expressions, “ambient temperature” and “room temperature,” as used herein, are understood in the art, and refer generally to a temperature, e.g., a reaction temperature, that is about the temperature of the room in which the reaction is carried out, e.g., a temperature from about 20° C. to about 30° C.

The present invention also includes pharmaceutically acceptable salts of the compounds described herein. The term “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the non-toxic salts of the parent compound formed, e.g., from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol or butanol) or acetonitrile (MeCN) are preferred. Lists of suitable salts are found in Remington's Pharmaceutical Sciences, 17^{th} Ed., (Mack Publishing Company, Easton, 1985), p. 1418, Berge et al., J. Pharm. Sci., 1977, 66(1), 1-19 and in Stahl et al., Handbook of Pharmaceutical Salts: Properties, Selection, and Use, (Wiley, 2002). In some embodiments, the compounds described herein include the N-oxide forms.

### II. Assays

Two separate screens to identify novel small molecule inhibitors of iRhom2/ADAM17 activity were developed. As demonstrated by genetic studies in mice, inactivation of iRhom2 in mice blocks the release of TNFα from bone marrow derived macrophages (McIlwain D R et al. (January 2012), “iRhom2 regulation of TACE controls TNF-mediated protection against Listeria and responses to LPS”, Science 335(6065): 229-32). Therefore, the primary screen for small molecule inhibitors of iRhom2/ADAM17 was a screen for inhibitors that blocked the release of TNFα from LPS-stimulated THP-1 human myeloid cells, a process that depends on iRhom2 and ADAM17. Since the release of TNFα from THP-1 cells can be blocked at many stages of the LPS/TLR4/iRhom2/ADAM17 pathway, a secondary screen was performed for another iRhom2/ADAM17 selective substrate, Kit-ligand 2 (KL-2) (Maretzky T et al. (July 2013), “iRhom2 controls the substrate selectivity of stimulated ADAM17-dependent ectodomain shedding”, PNAS 110(28): 11433-11438) in a different human cell type, human embryonic kidney cells (HEK 293).

A tertiary counterscreen was also performed to monitor the release of TGFα, which is a substrate of iRhom1/ADAM17 and iRhom2/ADAM17, so its release should not be blocked by an iRhom2-selective inhibitor (Maretzky T et al. (July 2013), “iRhom2 controls the substrate selectivity of stimulated ADAM117-dependent ectodomain shedding”, PNAS 110(28): 11433-11438; and Li X et al. (May 2015), “iRhoms 1 and 2 are essential upstream regulators of ADAM17-dependent EGFR signaling”, PNAS 112(19): 6080-6085).

Additional screens related to TNFα release in LPS-stimulated human THP-1 macrophage cells or LPS-stimulated human peripheral blood mononuclear cells (PBMCs) isolated from healthy donor were also performed to assess TNFα inhibition. Screens related to IL-6R release in PBMCs isolated from healthy donor were also performed to assess IL-6R inhibition. Screens related to HB-EGF release in PMA-stimulated human THP-1 macrophage cells or PMA-stimulated human PBMCs isolated from healthy donor were also performed to assess HB-EGF inhibition. Screens related to TGFα release in PMA-stimulated human PC3 prostate cancer cells were also performed to assess TGFα inhibition.

Assay conditions and the results with compounds of the disclosure are presented in the Examples.

### III. Uses of the Compounds

Compounds of the present disclosure can inhibit the function of iRhom2/ADAM17, or inhibit iRhom2/ADAM17 activity, and, thus, are useful in treating diseases and disorders associated with associated signaling pathways such as TNFα, IL-6 and EGFR. In some embodiments, the present disclosure provides a method for inhibiting the function of iRhom2/ADAM17, or inhibiting iRhom2/ADAM17 activity. The method includes administering to an individual or a patient a compound of any of the formulas as described herein, or of a compound as recited in any of the claims and described herein, or a pharmaceutically acceptable salt or a stereoisomer thereof. The compounds of the present disclosure can be used alone, in combination with other agents or therapies or as an adjuvant or neoadjuvant for the treatment of diseases or disorders, including cancer or infection diseases. For the uses described herein, any of the compounds of the disclosure, including any of the embodiments thereof, may be used.

In some embodiments, the present disclosure provides a method of treating a disease or disorder associated with inhibiting the function of iRhom2/ADAM17, or inhibition of iRhom2/ADAM17 activity. The method includes administering to the individual or patient in need thereof a therapeutically effective amount of a compound of any of the formulas as described herein, or of a compound as recited in any of the claims and described herein, or a salt or a stereoisomer thereof.

In some embodiments, the disease or disorder associated with inhibiting the function of iRhom2/ADAM17, or inhibition of iRhom2/ADAM17 activity, is traumatic brain injury. Traumatic brain injury (TBI) is a major cause of temporary or permanent cognitive impairment and disability. TBI can be triggered by any type of severe head trauma or impact, such as after a fall while bicycling, skiing, running or riding a motorcycle, following an automobile accident or other injuries, such as during combat. TBI leads to activation of immune cells in the brain that are called microglia. In response to such an injury, these cells, which are quiet and resting in a normal, healthy brain, become activated and release pro-inflammatory cytokines such as TNFα. Dysregulated release of TNFα, in turn, is known to cause cognitive impairment in mice, and presumably it has the same effect in human patients¹. Since iRhom2 is required for the release of TNFα from microglia², inhibiting the function of iRhom2/ADAM17, or inhibition of iRhom2/ADAM17 activity, should ameliorate or prevent some or all the consequences of TBI, including headaches, cognitive impairment, depression and dementia. In some embodiments, provided herein is a method for treating traumatic brain disorder. The method includes administering to a patient in need thereof, a therapeutically effective amount of a compound of the formulas as described herein, a compound as recited in any of the claims and described herein, or a salt thereof. The anticipated effect will be reduction or prevention of the symptoms of TBI.

In some embodiments, the disease or disorder associated with inhibiting the function of iRhom2/ADAM17, or inhibition of iRhom2/ADAM17 activity, is Alzheimer's Disease. Alzheimer's disease (AD) and the resulting dementia are devastating conditions that impact the lives of the affected individuals and their relatives and care givers. The ADAM17-dependent release of TNFα in immune cells is regulated by iRhom2². Interestingly, a recent study reported a highly significant association between changes in the methylation of iRhom2 (also referred to RHBDF2) and AD in humans³. iRhom2/ADAM17-dependent release of TNFα from microglia and brain leukocytes contributes to the neuroinflammatory stage of AD, so inhibiting the function of iRhom2/ADAM17, or inhibition of iRhom2/ADAM17 activity, could provide an attractive new target for treatment of AD. In some embodiments, provided herein is a method for treating Alzheimer's Disease. The method includes administering to a patient in need thereof, a therapeutically effective amount of a compound of the formulas as described herein, a compound as recited in any of the claims and described herein, or a salt thereof. The anticipated effect will be reduction or prevention of the symptoms of AD, reduced neuroinflammation and reduced brain damage, leading to increased quality of life and cognitive abilities compared to untreated patients.

In some embodiments, the disease or disorder associated with inhibiting the function of iRhom2/ADAM17, or inhibition of iRhom2/ADAM17 activity, is Hemophilic Arthropathy. Hemophilic arthropathy (HA) is one of the most serious consequences of bleeding disorders such as hemophilia A or B. HA is caused by bleeding into the joints of hemophilia patients and depending on the severity of the symptoms that develop over time, HA can have a devastating impact on patient lives⁴⁻⁷. Blood entering the joint can activate the iRhom2/ADAM17/TNFα signaling pathway, which causes joint erosion and damage as well as the osteoporosis that is known to affect HA patients⁸. Therefore, inhibitors of the function of iRhom2/ADAM17 or iRhom2/ADAM17 activity could function as a novel treatment of the joint damage and bone erosion that is associated with HA. In some embodiments, provided herein is a method for treating Hemophilic Arthropathy. The method includes administering to a patient in need thereof, a therapeutically effective amount of a compound of the formulas as described herein, a compound as recited in any of the claims and described herein, or a salt thereof. The anticipated effect will be reduction or prevention of joint erosion and damage and osteoporosis and osteopenia in patient suffering from HS, leading to improved quality of life and mobility in the affected patients. Compounds of the invention can be combined with other treatment of HA patients, such as replacement of Factor VIII, to enhance the effect of treatment and further increase the quality of life for the affected patients.

In some embodiments, the disease or disorder associated with inhibiting the function of iRhom2/ADAM17, or inhibition of iRhom2/ADAM17 activity, is Hemorrhagic Stroke. Hemorrhagic stroke (HS) is caused by bleeding into the brain. The primary consequence of HS is damage of brain tissue through the displacement caused by the bleeding, which has no outlet within the enclosed cavity of the skull. However, a secondary consequence is the resulting neuroinflammation, which is presumably a consequence of activation of microglia by blood and blood degradation products, in a similar manner as macrophages (which are very similar to microglia) can be activated in patients suffering from hemophilia arthropathy (see 8 and example 3). The activation of microglia will result in the release of TNFα, leading to negative sequelae such as cognitive impairment and dementia, as described above for AD and TBI. Because the production of TNFα that is triggered by blood in HA patients depends on iRhom2⁸, inhibiting the function of iRhom2/ADAM17, or inhibition of iRhom2/ADAM17 activity, is predicted to help prevent some or all of the devastating consequences of HS. In some embodiments, provided herein is a method for treating Hemorrhagic Stroke. The method includes administering to a patient in need thereof, a therapeutically effective amount of a compound of the formulas as described herein, a compound as recited in any of the claims and described herein, or a salt thereof. The anticipated effect will be reduction or prevention of the symptoms of HS, reduced neuroinflammation and reduced brain damage, leading to increased quality of life and cognitive abilities compared to untreated patients.

In some embodiments, the disease or disorder associated with inhibiting the function of iRhom2/ADAM17, or inhibition of iRhom2/ADAM17 activity, is Cytokine Storm, Macrophage Activation Syndrome. Cytokine Storm (CS) and macrophage activation syndrome (MAS) are thought to be crucial contributors to the pathogenesis of COVID-19 and other acute respiratory syndromes caused by Corona virus (CoV), Influenza virus and other acute insults to the lung. The viral infection or other causes of the cytokine storm result in an activation of the release of TNFα and the interleukin 6 receptor (IL-6R) from macrophages, which in turn results in an exacerbation of the disease and can lead to severe or even fatal outcomes for the affected patients. Since iRhom2 is required for the release of TNFα and the IL-6R from macrophages⁹⁻¹¹ (and data not shown re IL-6R), iRhom2 is an excellent target for treatment of CS/MAS. In some embodiments, provided herein is a method for treating Cytokine Storm, Macrophage Activation Syndrome. The method includes administering to a patient in need thereof, a therapeutically effective amount of a compound of the formulas as described herein, a compound as recited in any of the claims and described herein, or a salt thereof. The anticipated effect will be reduction of the CS/MAS, which in turn is predicted to significantly improve the outcome of acute respiratory syndrome and of other consequences of the CS/MAS, including damage to internal organs such as liver, kidney, heart and intestine.

In some embodiments, the disease or disorder associated with inhibiting the function of iRhom2/ADAM17, or inhibition of iRhom2/ADAM17 activity, is Rheumatoid Arthritis. Rheumatoid arthritis (RA) is a debilitating destructive inflammatory joint disease that affects about 0.5% to 1% of the population. TNFα and the IL-6/IL-6R pathway are currently considered excellent targets for treatment of RA. However, despite the success of these inhibitors of individual pro-inflammatory pathways, a significant number of patients treated with anti-TNF biologics (e.g., Humira, Etanercept) fail to respond and are then switched to IL-6 pathway inhibitors (e.g., Tocilizumab) and vice versa. Inhibitors of the function or activity of iRhom2/ADAM17 promise superior protection from RA because they block both the TNFα⁹⁻¹¹ and the IL-6R (data not shown re IL-6R) and the newly implicated HB-EGF/EGFR pathway¹² simultaneously. In some embodiments, provided herein is a method for treating Rheumatoid Arthritis. The method includes administering to a patient in need thereof, a therapeutically effective amount of a compound of the formulas as described herein, a compound as recited in any of the claims and described herein, or a salt thereof. The anticipated effect will be reduction of RA and superior protection by blocking all three disease causing pathways at the same time.

In some embodiments, the disease or disorder associated with inhibiting the function of iRhom2/ADAM17, or inhibition of iRhom2/ADAM17 activity, is Systemic Lupus Erythematosis-Glomerulonephritis. Systemic Lupus Erythematosis (SLE) is a prototypic autoimmune disease in which immune complex deposition leads to recruitment and activation of neutrophils and monocytes via Fcγ receptors (FcγR)¹³ and C5a receptors (C5aR). FcγR and complement play critical roles in immune complex-induced inflammation and subsequent organ damage. Engagement of FcγR and complement receptors on neutrophils (the first responders) and monocytes triggers production of reactive oxidants, release of proteolytic enzymes, phagocytosis, and upregulation of chemokines, cytokines, most prominently TNFα, and growth factors, including HB-EGF¹⁴. Studies in mice have shown that inactivation of iRhom2, which is required for the release of TNFα and HB-EGF from cells, protects from lethality and the severe glomerulonephritis (GN) caused in a mouse model of SLE¹⁵. Moreover, since patients suffering from SLE-GN also have dysregulated HB-EGF signaling¹⁴, which is caused by activation of iRhom2/ADAM17¹⁶, inhibitors of iRhom2/ADAM17 function or activity will be used to treat patients suffering from SLE-GN. In some embodiments, provided herein is a method for treating Systemic Lupus Erythematosis-Glomerulonephritis. The method includes administering to a patient in need thereof, a therapeutically effective amount of a compound of the formulas as described herein, a compound as recited in any of the claims and described herein, or a salt thereof. The anticipated effect will be reduction of SLE-GN and protection by blocking both disease-causing pathways at the same time (TNFα, HB-EGF).

It is believed that compounds of the disclosure, or any of the embodiments thereof, may possess satisfactory pharmacological profile and promising biopharmaceutical properties, such as toxicological profile, metabolism and pharmacokinetic properties, solubility, and permeability. It will be understood that determination of appropriate biopharmaceutical properties is within the knowledge of a person skilled in the art, e.g., determination of cytotoxicity in cells or inhibition of certain targets or channels to determine potential toxicity.

The terms “individual” or “patient,” used interchangeably, refer to any animal, including mammals, preferably mice, rats, other rodents, rabbits, dogs, cats, swine, cattle, sheep, horses, or primates, and most preferably humans.

The phrase “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein, the term “treating” or “treatment” refers to one or more of (1) inhibiting the disease; e.g., inhibiting a disease, condition or disorder in an individual who is experiencing or displaying the pathology or symptomatology of the disease, condition or disorder (i.e., arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, the compounds of the invention are useful in preventing or reducing the risk of developing any of the diseases referred to herein; e.g., preventing or reducing the risk of developing a disease, condition or disorder in an individual who may be predisposed to the disease, condition or disorder but does not yet experience or display the pathology or symptomatology of the disease.

### Combination Therapies

Compounds of the present disclosure, or pharmaceutically acceptable salts thereof, can be used in combination with one or more additional therapeutic agents for the treatment of diseases, such as Traumatic Brain Injury, Alzheimer's Disease, Hemorrhagic Stroke, Hemophilic Arthropathy, Cytokine Storm/Macrophage Activation Syndrome, Rheumatoid Arthritis, and Systemic Lupus Erythematosis-Glomerulonephritis.

When more than one pharmaceutical agent is administered to a patient, they can be administered simultaneously, separately, sequentially, or in combination (e.g., for more than two agents).

In some embodiments, the one or more additional therapeutic agents for the treatment of Hemophilic Arthropathy comprises coagulation factor replacement, e.g., FVIII replacement therapy.

In some embodiments, the one of more additional therapeutic agents for the treatment of Rheumatoid Arthritis comprises one or more agents selected from methotrexate, anti-TNF biologics or anti-IL-6 biologics.

### IV. Formulation, Dosage Forms and Administration

When employed as pharmaceuticals, the compounds of the present disclosure can be administered in the form of pharmaceutical compositions. Thus, the present disclosure provides a composition comprising a compound described herein, or a pharmaceutically acceptable salt thereof, or any of the embodiments thereof, and at least one pharmaceutically acceptable carrier or excipient. These compositions can be prepared in a manner well known in the pharmaceutical art, and can be administered by a variety of routes, depending upon whether local or systemic treatment is indicated and upon the area to be treated. Administration may be topical (including transdermal, epidermal, ophthalmic and to mucous membranes including intranasal, vaginal and rectal delivery), pulmonary (e.g., by inhalation or insufflation of powders or aerosols, including by nebulizer; intratracheal or intranasal), oral or parenteral. Parenteral administration includes intravenous, intraarterial, subcutaneous, intraperitoneal intramuscular or injection or infusion; or intracranial, e.g., intrathecal or intraventricular, administration. Parenteral administration can be in the form of a single bolus dose, or may be, e.g., by a continuous perfusion pump. Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable.

This disclosure also includes pharmaceutical compositions which contain, as the active ingredient, the compound of the present disclosure or a pharmaceutically acceptable salt thereof, in combination with one or more pharmaceutically acceptable carriers or excipients. In some embodiments, the composition is suitable for topical administration. In making the compositions of the disclosure, the active ingredient is typically mixed with an excipient, diluted by an excipient or enclosed within such a carrier in the form of, e.g., a capsule, sachet, paper, or other container. When the excipient serves as a diluent, it can be a solid, semi-solid, or liquid material, which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), ointments containing, e.g., up to 10% by weight of the active compound, soft and hard gelatin capsules, suppositories, sterile injectable solutions and sterile packaged powders.

In preparing a formulation, the active compound can be milled to provide the appropriate particle size prior to combining with the other ingredients. If the active compound is substantially insoluble, it can be milled to a particle size of less than 200 mesh. If the active compound is substantially water soluble, the particle size can be adjusted by milling to provide a substantially uniform distribution in the formulation, e.g., about 40 mesh.

The compounds of the disclosure may be milled using known milling procedures such as wet milling to obtain a particle size appropriate for tablet formation and for other formulation types. Finely divided (nanoparticulate) preparations of the compounds of the disclosure can be prepared by processes known in the art see, e.g., WO 2002/000196.

Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup and methyl cellulose. The formulations can additionally include: lubricating agents such as talc, magnesium stearate and mineral oil; wetting agents; emulsifying and suspending agents; preserving agents such as methyl- and propylhydroxy-benzoates; sweetening agents; and flavoring agents. The compositions of the disclosure can be formulated so as to provide quick, sustained or delayed release of the active ingredient after administration to the patient by employing procedures known in the art.

In some embodiments, the pharmaceutical composition comprises silicified microcrystalline cellulose (SMCC) and at least one compound described herein, or a pharmaceutically acceptable salt thereof. In some embodiments, the silicified microcrystalline cellulose comprises about 98% microcrystalline cellulose and about 2% silicon dioxide w/w.

In some embodiments, the composition is a sustained release composition comprising at least one compound described herein, or a pharmaceutically acceptable salt thereof, and at least one pharmaceutically acceptable carrier or excipient. In some embodiments, the composition comprises at least one compound described herein, or a pharmaceutically acceptable salt thereof, and at least one component selected from microcrystalline cellulose, lactose monohydrate, hydroxypropyl methylcellulose and polyethylene oxide. In some embodiments, the composition comprises at least one compound described herein, or a pharmaceutically acceptable salt thereof, and microcrystalline cellulose, lactose monohydrate and hydroxypropyl methylcellulose. In some embodiments, the composition comprises at least one compound described herein, or a pharmaceutically acceptable salt thereof, and microcrystalline cellulose, lactose monohydrate and polyethylene oxide. In some embodiments, the composition further comprises magnesium stearate or silicon dioxide. In some embodiments, the microcrystalline cellulose is Avicel PH102™. In some embodiments, the lactose monohydrate is Fast-flo 316™. In some embodiments, the hydroxypropyl methylcellulose is hydroxypropyl methylcellulose 2208 K4M (e.g., Methocel K4 M Premier™) and/or hydroxypropyl methylcellulose 2208 K100LV (e.g., Methocel K00LV™). In some embodiments, the polyethylene oxide is polyethylene oxide WSR 1105 (e.g., Polyox WSR 1105™).

In some embodiments, a wet granulation process is used to produce the composition. In some embodiments, a dry granulation process is used to produce the composition.

The compositions can be formulated in a unit dosage form, each dosage containing from about 5 to about 1,000 mg (1 g), more usually about 50 mg to about 400 mg, of the active ingredient. In some embodiments, each dosage contains about 50 mg of the active ingredient. In some embodiments, each dosage contains about 100 mg of the active ingredient. In some embodiments, each dosage contains about 200 mg of the active ingredient. In some embodiments, each dosage contains about 300 mg of the active ingredient. In some embodiments, each dosage contains about 400 mg of the active ingredient.

In some embodiments, the compound is administered to the patient at a daily dose in the range of about 50 mg/day to about 400 mg/day. In some embodiments, the compound is administered to the patient at a daily dose in the range of about 50 mg/day to about 300 mg/day, about 50 mg/day to about 300 mg/day, about 50 mg/day to about 200 mg/day, about 50 mg/day to about 100 mg/day, about 50 mg/day to about 75 mg/day, about 50 mg/day to about 60 mg/day, about 300 mg/day to about 400 mg/day, about 200 mg/day to about 400 mg/day, or about 100 mg/day to about 300 mg/day.

In some embodiments, the compound is administered to the patient at a daily dose of about 50 mg/day. In some embodiments, the compound is administered to the patient at a daily dose of about 100 mg/day. In some embodiments, the compound is administered to the patient at a daily dose of about 200 mg/day. In some embodiments, the compound is administered to the patient at a daily dose of about 300 mg/day. In some embodiments, the compound is administered to the patient at a daily dose of about 400 mg/day. In some embodiments, the compound is administered to the patient at a daily dose of about 500 mg/day. In some embodiments, the compound is administered to the patient at a daily dose of about 750 mg/day. In some embodiments, the compound is administered to the patient at a daily dose of about 1000 mg/day. In some embodiments, the compound is administered to the patient at a daily dose of about 10 mg/day. In some embodiments, the compound is administered to the patient at a daily dose of about 1 mg/day.

In some embodiments, the daily dose is in the range of about 1 mg/day to about 1000 mg/day, about 10 mg/day to about 750 mg/day, about 10 mg/day to about 500 mg/day, about 10 mg/day to about 400 mg/day, about 10 mg/day to about 300 mg/day, about 10 mg/day to about 200 mg/day, about 10 mg/day to about 100 mg/day, about 10 mg/day to about 50 mg/day, about 50 mg/day to about 500 mg/day, about 50 mg/day to about 400 mg/day, about 50 mg/day to about 300 mg/day, about 50 mg/day to about 200 mg/day, or about 50 mg/day to about 100 mg/day. In some aspects, the method includes administering to the patient a single dose of the composition. In some aspects, the method includes administering to the patient multiple doses of the composition. In some aspects, the method includes administering to the patient from 1 to 4 doses of the composition per day.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient.

The components used to formulate the pharmaceutical compositions are of high purity and are substantially free of potentially harmful contaminants (e.g., at least National Food grade, generally at least analytical grade, and more typically at least pharmaceutical grade). Particularly for human consumption, the composition is preferably manufactured or formulated under Good Manufacturing Practice standards as defined in the applicable regulations of the U.S. Food and Drug Administration. For example, suitable formulations may be sterile and/or substantially isotonic and/or in full compliance with all Good Manufacturing Practice regulations of the U.S. Food and Drug Administration.

The active compound may be effective over a wide dosage range and is generally administered in a therapeutically effective amount. It will be understood, however, that the amount of the compound actually administered will usually be determined by a physician, according to the relevant circumstances, including the condition to be treated, the chosen route of administration, the actual compound administered, the age, weight, and response of the individual patient, the severity of the patient's symptoms and the like.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. In some embodiments, the dose range is from about 0.02 mg/kg to about 20 mg/kg, about 0.05 mg/kg to about 10 mg/kg, 0.1 mg/kg to about 10 mg/kg, 0.2 mg/kg to about 8 mg/kg, 0.5 mg/kg to about 5 mg/kg, 1 mg/kg to about 5 mg/kg, or 2 mg/kg to about 3 mg/kg of body weight per day. In some embodiments, the dose is about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg of body weight per day.

The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

For preparing solid compositions such as tablets, the principal active ingredient is mixed with a pharmaceutical excipient to form a solid preformulation composition containing a homogeneous mixture of a compound of the present disclosure. When referring to these preformulation compositions as homogeneous, the active ingredient is typically dispersed evenly throughout the composition so that the composition can be readily subdivided into equally effective unit dosage forms such as tablets, pills and capsules. This solid preformulation is then subdivided into unit dosage forms of the type described above containing from, e.g., about 0.1 to about 1000 mg of the active ingredient of the present disclosure.

The tablets or pills of the present disclosure can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permit the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the compounds and compositions of the present disclosure can be incorporated for administration orally or by injection include aqueous solutions, suitably flavored syrups, aqueous or oil suspensions, and flavored emulsions with edible oils such as cottonseed oil, sesame oil, coconut oil, or peanut oil, as well as elixirs and similar pharmaceutical vehicles.

Compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as described supra. In some embodiments, the compositions are administered by the oral or nasal respiratory route for local or systemic effect. Compositions can be nebulized by use of inert gases. Nebulized solutions may be breathed directly from the nebulizing device or the nebulizing device can be attached to a face mask, tent, or intermittent positive pressure breathing machine. Solution, suspension, or powder compositions can be administered orally or nasally from devices which deliver the formulation in an appropriate manner.

Topical formulations can contain one or more conventional carriers. In some embodiments, ointments can contain water and one or more hydrophobic carriers selected from, e.g., liquid paraffin, polyoxyethylene alkyl ether, propylene glycol, white Vaseline, and the like. Carrier compositions of creams can be based on water in combination with glycerol and one or more other components, e.g., glycerinemonostearate, PEG-glycerinemonostearate and cetylstearyl alcohol. Gels can be formulated using isopropyl alcohol and water, suitably in combination with other components such as, e.g., glycerol, hydroxyethyl cellulose, and the like. In some embodiments, topical formulations contain at least about 0.1, at least about 0.25, at least about 0.5, at least about 1, at least about 2 or at least about 5 wt. % of the compound of the disclosure. The topical formulations can be suitably packaged in tubes of, e.g., 100 g which are optionally associated with instructions for the treatment of the select indication, e.g., psoriasis or other skin condition.

The amount of compound or composition administered to a patient will vary depending upon what is being administered, the purpose of the administration, such as prophylaxis or therapy, the state of the patient, the manner of administration and the like. In therapeutic applications, compositions can be administered to a patient already suffering from a disease in an amount sufficient to cure or at least partially arrest the symptoms of the disease and its complications. Effective doses will depend on the disease condition being treated as well as by the judgment of the attending clinician depending upon factors such as the severity of the disease, the age, weight and general condition of the patient and the like.

The compositions administered to a patient can be in the form of pharmaceutical compositions described above. These compositions can be sterilized by conventional sterilization techniques, or may be sterile filtered. Aqueous solutions can be packaged for use as is, or lyophilized, the lyophilized preparation being combined with a sterile aqueous carrier prior to administration. The pH of the compound preparations typically will be between 3 and 11, more preferably from 5 to 9 and most preferably from 7 to 8. It will be understood that use of certain of the foregoing excipients, carriers or stabilizers will result in the formation of pharmaceutical salts.

The therapeutic dosage of a compound of the present disclosure can vary according to, e.g., the particular use for which the treatment is made, the manner of administration of the compound, the health and condition of the patient, and the judgment of the prescribing physician. The proportion or concentration of a compound of the disclosure in a pharmaceutical composition can vary depending upon a number of factors including dosage, chemical characteristics (e.g., hydrophobicity), and the route of administration. For example, the compounds of the disclosure can be provided in an aqueous physiological buffer solution containing about 0.1 to about 10% w/v of the compound for parenteral administration. Some typical dose ranges are from about 1 μg/kg to about 1 g/kg of body weight per day. In some embodiments, the dose range is from about 0.01 mg/kg to about 100 mg/kg of body weight per day. The dosage is likely to depend on such variables as the type and extent of progression of the disease or disorder, the overall health status of the particular patient, the relative biological efficacy of the compound selected, formulation of the excipient, and its route of administration. Effective doses can be extrapolated from dose-response curves derived from in vitro or animal model test systems.

### V. Labeled Compounds and Assay Methods

The compounds of the present disclosure can further be useful in investigations of biological processes in normal and abnormal tissues. Thus, another aspect of the present disclosure relates to labeled compounds of the disclosure (radio-labeled, fluorescent-labeled, etc.) that would be useful not only in imaging techniques but also in assays, both in vitro and in vivo, for localizing and quantitating iRhom2 in tissue samples, including human, and for identifying iRhom2 ligands by inhibition binding of a labeled compound. Accordingly, the present disclosure includes iRhom2 binding assays that contain such labeled compounds.

The present disclosure further includes isotopically-labeled compounds of the disclosure. An “isotopically” or “radio-labeled” compound is a compound of the disclosure where one or more atoms are replaced or substituted by an atom having an atomic mass or mass number different from the atomic mass or mass number typically found in nature (i.e., naturally occurring). Suitable radionuclides that may be incorporated in compounds of the present disclosure include but are not limited to ³H (also written as T for tritium), ¹¹C, ¹³C, ¹⁴C, ¹³N, ¹⁵N, ¹⁵O, ¹⁷O, ¹⁸O, ¹⁸F, ³⁵S, ³⁶Cl, ⁸²Br, ⁷⁵Br, ⁷⁶Br, ⁷⁷Br, ¹²³I, ¹²⁴I, ¹²⁵I and ¹³¹I. For example, one or more hydrogen atoms in a compound of the present disclosure can be replaced by deuterium atoms.

One or more constituent atoms of the compounds presented herein can be replaced or substituted with isotopes of the atoms in natural or non-natural abundance. In some embodiments, the compound includes at least one deuterium atom. In some embodiments, the compound includes two or more deuterium atoms. In some embodiments, the compound includes 1-2, 1-3, 1-4, 1-5, or 1-6 deuterium atoms. In some embodiments, all of the hydrogen atoms in a compound can be replaced or substituted by deuterium atoms.

Synthetic methods for including isotopes into organic compounds are known in the art (Deuterium Labeling in Organic Chemistry by Alan F. Thomas (New York, N.Y., Appleton-Century-Crofts, 1971; The Renaissance of H/D Exchange by Jens Atzrodt, Volker Derdau, Thorsten Fey and Jochen Zimmermann, Angew. Chem. Int. Ed. 2007, 7744-7765; The Organic Chemistry of Isotopic Labelling by James R. Hanson, Royal Society of Chemistry, 2011). Isotopically labeled compounds can be used in various studies such as NMR spectroscopy, metabolism experiments, and/or assays.

Substitution with heavier isotopes, such as deuterium, may afford certain therapeutic advantages resulting from greater metabolic stability, for example, increased in vivo half-life or reduced dosage requirements, and hence may be preferred in some circumstances. (see e.g., A. Kerekes et al. J. Med. Chem. 2011, 54, 201-210; R. Xu et al. J. Label Compd. Radiopharm. 2015, 58, 308-312). In particular, substitution at one or more metabolism sites may afford one or more of the therapeutic advantages.

The radionuclide that is incorporated in the instant radio-labeled compounds will depend on the specific application of that radio-labeled compound. For example, for in vitro PD-L1 protein labeling and competition assays, compounds that incorporate ³H, ¹⁴C, ⁸²Br, ¹²⁵I, ¹³¹I, ³⁵S or will generally be most useful. For radio-imaging applications ¹¹C, ¹⁸F, ¹²⁵I, ¹²³I, ¹²⁴I, ¹³¹I, ⁷⁵Br, ⁷⁶Br or ⁷⁷Br can be useful.

It is understood that a “radio-labeled” or “labeled compound” is a compound that has incorporated at least one radionuclide. In some embodiments, the radionuclide is selected from the group consisting of ³H, ¹⁴C, ¹²⁵I, ³⁵S and ⁸²Br.

The present disclosure can further include synthetic methods for incorporating radio-isotopes into compounds of the disclosure. Synthetic methods for incorporating radio-isotopes into organic compounds are well known in the art, and a person of ordinary skill in the art will readily recognize the methods applicable for the compounds of disclosure.

A labeled compound of the disclosure can be used in a screening assay to identify and/or evaluate compounds. For example, a newly synthesized or identified compound (i.e., test compound) which is labeled can be evaluated for its ability to bind iRhom2 by monitoring its concentration variation when contacting with iRhom2, through tracking of the labeling. For example, a test compound (labeled) can be evaluated for its ability to reduce binding of another compound which is known to bind to iRhom2 (i.e., standard compound). Accordingly, the ability of a test compound to compete with the standard compound for binding to iRhom2 protein directly correlates to its binding affinity. Conversely, in some other screening assays, the standard compound is labeled and test compounds are unlabeled. Accordingly, the concentration of the labeled standard compound is monitored in order to evaluate the competition between the standard compound and the test compound, and the relative binding affinity of the test compound is thus ascertained.

### VI. Kits

The present disclosure also includes pharmaceutical kits useful, e.g., in the treatment or prevention of diseases or disorders associated with the activity of iRhom2/ADAM17, which include one or more containers containing a pharmaceutical composition comprising a therapeutically effective amount of a compound disclosed herein. Such kits can further include one or more of various conventional pharmaceutical kit components, such as, e.g., containers with one or more pharmaceutically acceptable carriers, additional containers, etc., as will be readily apparent to those skilled in the art. Instructions, either as inserts or as labels, indicating quantities of the components to be administered, guidelines for administration, and/or guidelines for mixing the components, can also be included in the kit.

The following abbreviations may be used herein: AD (Alzheimer's Disease); AP (alkaline phosphatase); CS/MAS (Cytokine Storm and Macrophage Activation Syndrome); DMSO (dimethylsulfoxide); DNA (deoxyribonucleic acid); g (gram(s)); HA (Hemophilic Arthropathy); HB-EGF (heparin-binding-epidermal growth factor); HEK (human embryonic kidney); HS (Hemorrhagic Stroke); HTRF (homogeneous time resolved fluorescence); IC₅₀ (concentration needed to reach 50% of inhibition of activity); kg (kilogram(s)); KL-2 or KitL2 (Kit-ligand-2); LPS (lipopolysaccharide); M (molar); mg (milligram(s)); min. (minutes(s)); mL (milliliter(s)); mM (millimolar); NaOH (sodium hydroxide); nL (nanoliter(s)); nM (nanomolar); μg (microgram(s)); μL (microliter(s)); μM (micromolar); PMA (phorbol 12-myristate 13-acetate); pNP (para-nitrophenylphenol); pNPP (para-nitrophenylphosphate); RA (rheumatoid arthritis); RT (room temperature); SLE-GN (Systemic Lupus Erythematosis-Glomerulonephritis); TBI (Traumatic Brain Injury); TNFα (tumor necrosis factor alpha); XC₅₀ (concentration needed to reach 50% of inhibition of activity).

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results. The compounds of the disclosure have been found to inhibit iRhom2/ADAM17 activity according to at least one assay described herein.

### REFERENCES CITED

1. Sriram K, O'Callaghan J P. Divergent roles for tumor necrosis factor-alpha in the brain. J Neuroimmune Pharmacol. 2007; 2(2):140-153.
2. Li X, Maretzky T, Weskamp G, et al. iRhoms 1 and 2 are essential upstream regulators of ADAM17-dependent EGFR signaling. Proc Natl Acad Sci USA. 2015; 112(19):6080-6085.
3. De Jager P L, Srivastava G, Lunnon K, et al. Alzheimer's disease: early alterations in brain DNA methylation at ANK1, BIN1, RHBDF2 and other loci. Nat Neurosci. 2014; 17(9):1156-1163.
4. Simpson M L, Valentino L A. Management of joint bleeding in hemophilia. Expert Rev Hematol. 2012; 5(4):459-468.
5. Stephensen D, Rodriguez-Merchan E C. Orthopaedic co-morbidities in the elderly haemophilia population: a review. Haemophilia. 2013; 19(2):166-173.
6. Haxaire C, Blobel C P. With blood in the joint—what happens next? Could activation of a pro-inflammatory signalling axis leading to iRhom2/TNFalpha-convertase-dependent release of TNFalpha contribute to haemophilic arthropathy? Haemophilia. 2014; 20 Suppl 4:11-14.
7. Blobel C, Haxaire C, Kalliolias G, DiCarl E, Salmon J, Srivastava A. Blood induced arthropathy in hemophilia—Mechanisms and heterogeneity. Seminars in Thrombosis and Hemostasis. 2015; (in press).
8. Haxaire C, Hakobyan N, Pannellini T, et al. Blood-induced bone loss in murine hemophilic arthropathy is prevented by blocking the iRhom2/ADAM17/TNF-alpha pathway. Blood. 2018; 132(10):1064-1074.
9. McIlwain D R, Lang P A, Maretzky T, et al. iRhom2 regulation of TACE controls TNF-mediated protection against Listeria and responses to LPS. Science. 2012; 335(6065):229-232.
10. Adrain C, Zettl M, Christova Y, Taylor N, Freeman M. Tumor necrosis factor signaling requires iRhom2 to promote trafficking and activation of TACE. Science. 2012; 335(6065):225-228.
11. Issuree P D, Maretzky T, McIlwain D R, et al. iRHOM2 is a critical pathogenic mediator of inflammatory arthritis. J Clin Invest. 2013; 123(2):928-932.
12. Kuo D, Ding J, Cohn I S, et al. HBEGF(+) macrophages in rheumatoid arthritis induce fibroblast invasiveness. Sci Transl Med. 2019; 11(491).
13. Nimmerjahn F, Ravetch J V. Fcgamma receptors as regulators of immune responses. Nat Rev Immunol. 2008; 8(1):34-47.
14. Bollee G, Flamant M, Schordan S, et al. Epidermal growth factor receptor promotes glomerular injury and renal failure in rapidly progressive crescentic glomerulonephritis. Nat Med. 2011; 17(10):1242-1250.
15. Qing X, Chinenov Y, Redecha P, et al. iRhom2 promotes lupus nephritis through TNF-alpha and EGFR signaling. J Clin Invest. 2018; 128(4):1397-1412.
16. Maretzky T, McIlwain D R, Issuree P D, et al. iRhom2 controls the substrate selectivity of stimulated ADAM17-dependent ectodomain shedding. Proc Natl Acad Sci USA. 2013; 110(28):11433-11438.

### EXAMPLES

### Example 1a. Primary Screen: LPS/PMA-Stimulated TNFα Release from THP-1 Cells

THP-1 cells were plated in 384 or 1,536 wells and transferred on the ultra-high throughput screening (uHTS) platform Mark III, and TNFα shedding was initiated by LPS stimulation of THP-1 cells. The extent of released TNFα was detected with the corresponding HTRF antibodies labeled with europium cryptate (donor) and d2 (acceptor). The HTRF signal was generated by proximity of europium cryptate and d2. All detection reagents were purchased from Cisbio. TNFα detection was performed according to the manufacturer's protocol (Product Insert for Cisbio TNFα (h) Kit Part #62HTNFAPEG & 62HTNFAPEH; https://www.cisbio.com/media/asset/c/i/cisbio_dd_pi_62htnfapeg-62htnfapeh.pdf, accessed Sep. 28, 2020).

7.5 nL compound and controls in DMSO [10 μM in assay; 0.25% DMSO in 3 μL Assay] were dissolved in 2 μL cell suspension [2.4E6/mL; 4800 cells/well; culture w/o PenStrep], pre-incubated at 37° C., 5% CO₂, for 15 min with 1 μL LPS [100 ng/mL in 3 μL assay]. The cells were incubated at 37° C., 5% CO₂, for 3 h with 2 μL of the HTRF-mix [1:300 f.c.]. After an incubation at room temperature for 2 h or longer, the HTRF readout was performed. Then data evaluation for normalization against the 50 μM Batimastat control (=100% activation) was applied. As an additional control, 300 nM Batimastat was used to monitor performance of cells and assay sensitivity over time.

### Example 1b. Primary Screen: LPS/PMA-Stimulated TNFα Release from THP-1 Cells

THP-1 cells were plated in low volume 384 wells (10,000 cells per well in 12.6 μL RPMI medium) together with inhibitors or 10 μM BB94 and incubated over night. The next day, TNFα shedding was initiated by stimulation of THP-1 cells with 100 ng/mL LPS (1.8 μL of an 800 ng/mL LPS stock) for 3 hrs at 37° C. The extent of released TNFα was detected with the corresponding HTRF antibodies labeled with europium cryptate (donor) and d2 (acceptor). The HTRF signal was generated by proximity of europium cryptate and d2. All detection reagents were purchased from Cisbio. TNFα detection was performed according to the manufacturer's protocol (Product Insert for Cisbio TNFα (h) Kit Part #62HTNFAPEG & 62HTNFAPEH; https://www.cisbio.com/media/asset/c/i/cisbio_dd_pi_62htnfapeg-62htnfapeh.pdf, accessed Sep. 28, 2020).

Low volume 384 well plates were pre-coated with small molecule library compounds for a final concentration of 20 μM in a 15 μL reaction, or 0.75 μL of 200 μM stock of BB94 in 5% DMSO (final concentration 0.25%). 12.6 μL of cell suspension [8E5/mL; 10,000 cells/well; culture RPMI medium] and pre-incubated at 37° C., 5% CO₂, over night. The next day, 1.8 μL of an 800 ng/mL LPS stock was added [final concentration 100 ng/mL in 15 μL assay] and the cells were incubated at 37° C., 5% CO₂, for 3 h. Subsequently, 3 μL of the HTRF-mix [used at 1:3 dilution of cisbio HTRF human TNF] was added and the plates were incubated for an additional 2 hrs at 25° C. After an incubation at room temperature for 2 h, the HTRF readout was performed. Then data evaluation for normalization against the 10 μM Batimastat control (=100% activation) was applied.

### Example 2a. Cell Generation for Examples 3a and 4a (Secondary Screen and Counter Screen)

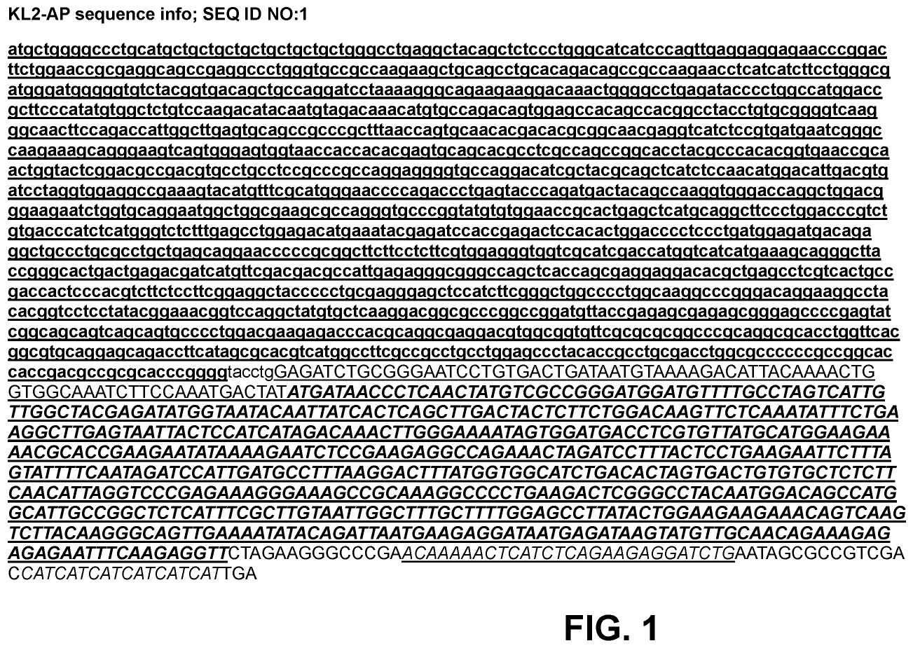

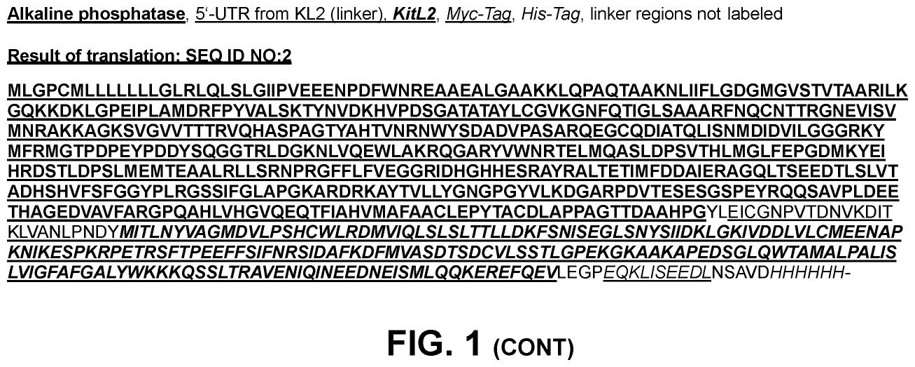

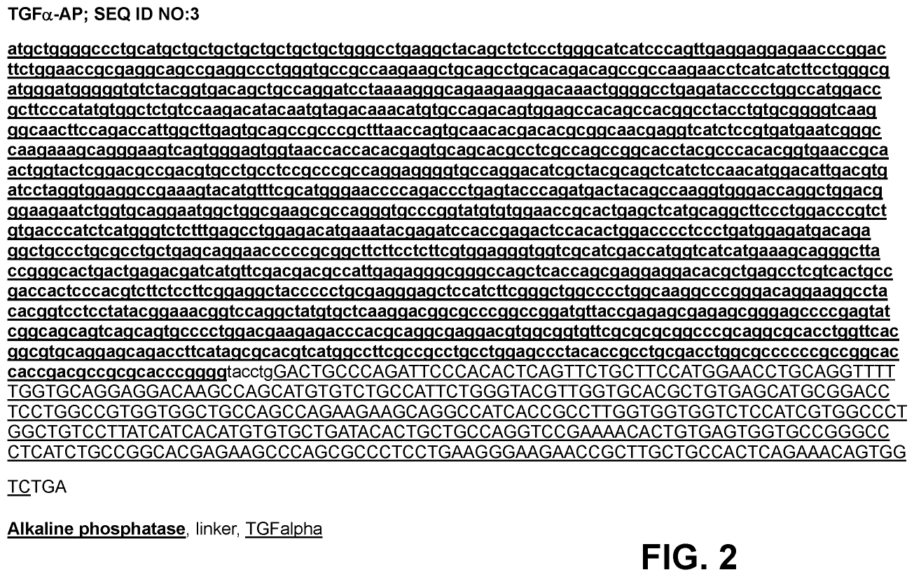

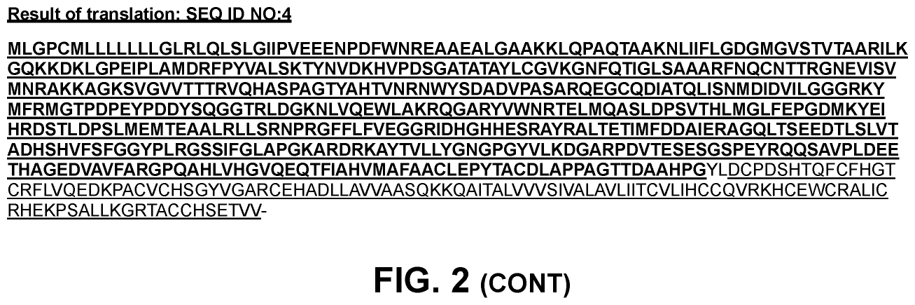

First, expression plasmids for alkaline phosphatase (AP)-fused KL2 and TGFα were designed and generated through gene synthesis followed by subcloning into the pcDNA3.1(+)/Hygro expression vector. The sequence for KL2-AP (SEQ ID NO:1) and the result of translation (SEQ ID NO:2) are displayed in FIG. 1. The sequence for TGFα-AP (SEQ ID NO:3) and the result of translation (SEQ ID NO:4) are displayed in FIG. 2. Subsequently, DNA amplifications were performed to obtain sufficient amounts of the expression vectors. The quality of DNA and gene sequences were confirmed through restriction digest and Sanger sequencing. In parallel, cell culture of THP-1 and HEK-293 cells was initiated. Master and working cell banks were prepared. Both cell lines were scaled up and transfected with the respective constructs by electroporation. During cell culturing, cell density and viability were monitored to ensure optimal conditions for the transfections. After PMA stimulation, AP-coupled KL2 or TGFα were detected by measurement of AP activity in the supernatant. Initial experiments regarding TGFα shedding showed a good performance of HEK-293. In contrast, THP-1 cells turned out to be not a suitable transfection host resulting in a loss of cell viability after transfection. The decision was made to proceed with HEK-293 for both selectivity assays and to establish polyclonal cell lines stably expressing AP-coupled KL2 or TGFα.

After electroporation, cells were further cultured in presence of selection antibiotics, hygromycin B, to generate stably transfected polyclonal cell pools. For both assays, KL2 and TGFα shedding, the respective selected pool turned out to be suitable. Final assay conditions were determined for the selected pools. For all experiments, batimastat (30 μM) was used as positive control for full inhibition of KL2 or TGFα shedding.

### Example 2b. Cell Generation for Examples 3b and 4b (Secondary Screen and Counter Screen)

Expression plasmids for alkaline phosphatase (AP)-fused human KL2 and TGFα had been previously designed and generated through gene synthesis followed by subcloning into the pcDNA3.1(+)/Hygro expression vector. The sequence for KL2-AP (SEQ ID NO:1) and the result of translation (SEQ ID NO:2) are displayed in FIG. 1. The sequence for TGFα-AP (SEQ ID NO:3) and the result of translation (SEQ ID NO:4) are displayed in FIG. 2. Subsequently, DNA amplifications were performed to obtain sufficient amounts of the expression vectors. The quality of DNA and gene sequences were confirmed through restriction digest and Sanger sequencing. In parallel, cell culture of HEK-293 cells was initiated. Master and working cell banks were prepared, scaled up and transfected with the respective constructs by electroporation. During cell culturing, cell density and viability were monitored to ensure optimal conditions for the transfections. After PMA stimulation, AP-coupled KL2 or TGFα were detected by measurement of AP activity in the supernatant.

After electroporation, cells were further cultured in presence of selection antibiotics, hygromycin B, to generate stably transfected polyclonal cell pools. For both assays, KL2 and TGFα shedding, the respective selected pool turned out to be suitable. Final assay conditions were determined for the selected pools. For all experiments, batimastat (BB94, 10 μM) was used as positive control for full inhibition of KL2 or TGFα shedding.

### Example 3a. Secondary Screen: PMA-Stimulated KL2 Release from HEK-293 Cells

60 μL of cells (40,000/well) were added to a sterile 384-well plate. After incubation overnight at 37° C. (5% CO₂), 50 μL medium were removed and 20 μL of prediluted compounds were added to the cells. After incubation for 15 min, 20 μL of PMA (500 ng/mL final conc.) were added. The cells were incubated for 2 h at 37° C. (5% CO₂). Then, 20 μL of the supernatant was transferred to a fresh plate and 20 μL of pNPP (5 mM final conc.) was added. AP reaction was performed for 1 h at RT. The reaction was stopped by addition of 20 μL of NaOH (1 M final conc.) and the absorbance of pNP was measured at 405 nm.

### Example 3b. Secondary Screen: PMA-Stimulated KL2 Release from HEK-293 Cells

The wells of sterile 384-well flat bottom clear plates for KL2-AP assays were coated with 10 μL of 0.1 mg/mL poly-d-lysine for 3-4 hr at 25° C. or overnight at 4° C., then washed 2× with PBS and patted dry. 60 μL of cells (40,000/well) were added to the sterile 384-well plate and incubated in Optimem (with 2% FCS and 1% Pen-strep) overnight at 37° C. (5% CO₂). After incubation overnight, 55 μL medium were removed with a BioTek EL406 and 10 μL of prediluted compounds were added to the cells for a final concentration of 20 μM. After incubation for 15 min, 15 μL of PMA (100 ng/mL final conc.) were added. The cells were incubated for 2 h at 37° C. (5% CO₂). Then, the plates were centrifuged for 5 min at 1000 rpm and 3 μL of the supernatant was transferred to a fresh 384 well plate and 9 μL of AP Balance Buffer was added per well. 12 μL of pNPP p-nitrophenyl phosphate (final 1 M pNPP) was added. AP reaction was performed for 1 h at 37° C. The reaction was stopped by addition of 12 μL of NaOH (1 M final conc.), the plates were centrifuged to remove air bubbles and the absorbance of pNP was measured at 405 nm.

### Example 4a. Counter Screen: PMA-Stimulated TGFα Release from HEK-293 Cells

60 μL of cells (20,000/well) were added to a sterile 384-well plate. After incubation overnight at 37° C. (5% CO₂), 50 μL medium were removed and 20 μL of prediluted compounds were added to the cells. After incubation for 15 min, 20 μL of PMA (100 ng/mL final conc.) were added. The cells were incubated for 2 h at 37° C. (5% CO₂). Then, 20 μL of the supernatant was transferred to a fresh plate and 20 μL of pNPP (5 mM final conc.) was added. The AP reaction was performed for 1 h at RT. The reaction was stopped by addition of 20 μL of NaOH (1 M final conc.) and the absorbance of pNP was measured at 405 nm.

### Example 4b. Counter Screen: PMA-Stimulated TGFα Release from HEK-293 Cells

The wells of sterile 384-well flat bottom clear plates for TGF-AP assays were coated with 10 μL of 0.1 mg/mL poly-d-lysine for 3-4 hr at 25° C. or overnight at 4° C., then washed 2× with PBS and patted dry. 60 μL of cells (20,000/well) were added to the sterile 384-well plate and incubated in Optimem (with 2% FCS and 1% Pen-strep) overnight at 37° C. (5% CO₂). After incubation overnight, 55 μL medium were removed with a BioTek EL406 and 10 μL of prediluted compounds were added to the cells for a final concentration of 20 μM. After incubation for 15 min, 15 μL of PMA (100 ng/mL final conc.) were added. The cells were incubated for 2 h at 37° C. (5% CO₂). Then, the plates were centrifuged for 5 min at 1000 rpm and 12 μL of the supernatant was transferred to a fresh 384 well plate. 12 μL of pNPP p-nitrophenyl phosphate (final 1 M pNPP) was added. AP reaction was performed for 1 h at 37° C. The reaction was stopped by addition of 12 μL of NaOH (1 M final conc.), the plates were centrifuged to remove air bubbles and the absorbance of pNP was measured at 405 nm.

### Example 5. Treatment of Traumatic Brain Injury

A patient suffering from Traumatic Brain Injury (TBI) is treated with 1 to 400 mg/day of a compound of this invention, e.g., 50 to 400 mg/day, in capsule or tablet form either as a single or divided dose. The anticipated effect will be reduction or prevention of the symptoms of TBI.

### Example 6. Treatment of Alzheimer's Disease

A patient suffering from Alzheimer's Disease (AD) or determined to be at risk for AD, either based on genetic predisposition or predictive cognitive tests or based on biomarkers of disease, is treated with 1 to 400 mg/day of a compound of this invention, e.g., 50 to 400 mg/day of a compound of this invention, in capsule or tablet form either as a single or divided dose. The anticipated effect will be reduction or prevention of the symptoms of AD, reduced neuroinflammation and reduced brain damage, leading to increased quality of life and cognitive abilities compared to untreated patients.

### Example 7. Treatment of Hemophilic Arthropathy

A patient suffering from Hemophilic Arthropathy (HA) or from acute or chronic intraarticular bleeding episodes is treated with 1 to 400 mg/day of a compound of this invention, e.g., 50 to 400 mg/day, in capsule or tablet form either as a single or divided dose. The anticipated effect will be reduction or prevention of joint erosion and damage and osteoporosis and osteopenia in patient suffering from HA, leading to improved quality of life and mobility in the affected patients. Inhibitors of iRhom2/ADAM17 activity can be combined with other treatment of HA patients, such as replacement of Factor VIII, to enhance the effect of treatment and further increase the quality of life for the affected patients.

### Example 8. Treatment of Hemorrhagic Stroke

A patient suffering from Hemorrhagic Stroke (HS) is treated with 1 to 400 mg/day of a compound of this invention, e.g., 50 to 400 mg/day, in capsule or tablet form either as a single or divided dose. The anticipated effect will be reduction or prevention of the symptoms of HS, reduced neuroinflammation and reduced brain damage, leading to increased quality of life and cognitive abilities compared to untreated patients.

### Example 9. Treatment of Cytokine Storm and Macrophage Activation Syndrome

A patient suffering from Cytokine Storm and Macrophage Activation Syndrome (CS/MAS) is treated with 1 to 400 mg/day of a compound of this invention, e.g., 50 to 400 mg/day, in capsule or tablet form either as a single or divided dose. The anticipated effect will be reduction of the CS/MAS, which in turn is predicted to significantly improve the outcome of acute respiratory syndrome and of other consequences of the CS/MAS, including damage to internal organs such as liver, kidney, heart and intestine.

### Example 10. Treatment of Rheumatoid Arthritis

A patient suffering from Rheumatoid Arthritis (RA) is treated with 1 to 400 mg/day of a compound of this invention, e.g., 50 to 400 mg/day, in capsule or tablet form either as a single or divided dose. The anticipated effect will be reduction of RA and superior protection by blocking all three disease causing pathways at the same time.

### Example 11. Treatment of Systemic Lupus Erythematosis-Glomerulonephritis

A patient suffering from Systemic Lupus Erythematosis-Glomerulonephritis (SLE-GN) is treated with 1 to 400 mg/day of a compound of this invention, e.g., 50 to 400 mg/day, in capsule or tablet form either as a single or divided dose. The anticipated effect will be reduction of SLE-GN and protection by blocking both disease-causing pathways at the same time (TNFα, HB-EGF).

Results from Examples 1a, 3a and 4a for Compounds of the Disclosure

Compounds of the disclosure were assessed in each of the primary screen (Example 1a), secondary screen (Example 3a), and counter screen (Example 4a), and the results are shown in Table 2.

TABLE 2

| | Primary Screen | Secondary Screen | Counter Screen |
|---|---|---|---|
| Compound | XC₅₀ TNFα (μM) | XC₅₀ KL2 (μM) | XC₅₀ TGFα (μM) |
| 1 | <0.802 | >128 | 12.6 |
| 2 | <0.802 | >128 | 9.91 |

Results from Examples 1b, 3b and 4b for Compounds of the Disclosure

Compounds of the disclosure were assessed in each of the primary screen (Example 1b), secondary screen (Example 3b), and counter screen (Example 4b), and the results are shown in Table 3.

TABLE 3

| | Primary Screen | Secondary Screen | Counter Screen |
|---|---|---|---|
| Compound | IC₅₀ TNFα (μM) | IC₅₀ KitL2 (μM) | IC₅₀ TGFα (μM) |
| 3 | <0.0625 | >40.0 | >40.0 |
| 4 | 7.57 | >40.0 | >40.0 |
| 5 | 5.21 | >40.0 | >40.0 |
| 6 | 4.86 | >40.0 | >40.0 |
| 7 | 3.93 | 22 | >40.0 |

### Example 12: Analysis of Inhibitory Effects of the Compounds of the Invention on LPS-Induced TNFα Shedding in Human THP-1 Macrophage Cells In Vitro

In the following study, ELISA-based TNFα release assay was performed to verify the inhibitory effects of Compounds 1, 2, 3, 4, 5, and 7 on LPS-induced release of endogenous TNFα from human THP-1 macrophage cells. The ELISA-based TNFα release assay that was used in this example is described below.

In brief, on day 1, Nunc black MaxiSorp® 96-well plates (Thermo Fisher Scientific, USA) were coated overnight with 100 μl per well of mouse anti-human TNFα capture antibody (provided as part of the DuoSet ELISA kit) at 4 μg/ml TBS at 4° C. On day 2, the capture antibody solution was removed and MaxiSorp® plates were blocked with 300 μl per well of TBS, 1% BSA at room temperature for 3 hours. Meanwhile, 20,000 THP-1 (American Type Culture Collection, USA) cells in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), compounds of the invention at 50 μM (for a final concentration of 10 μM in the resulting 100 μl sample volume) and in case of Buffer stimulated and unstimulated controls with DMSO (Carl Roth, Germany) at 50 μM (for a final concentration of 10 μM in the resulting 100 μl sample volume) at 37° C., 5% CO₂ for 30 minutes. Subsequently, cells (except those for unstimulated controls) were stimulated with 20 μl per well of LPS (Sigma-Aldrich, USA) at 300 ng/ml growth medium for a final concentration of 50 ng/ml at 37° C., 5% CO₂ for 2 hours. Afterwards, the 96-well plates were centrifuged to pellet cells. In parallel, blocking buffer was removed from the MaxiSorp® plates and plates were washed 4 times with 350 μl per well of TBS-T (Carl Roth, Germany) on a 96-head plate washer (Tecan Group, Switzerland). To avoid drying-up, 30 μl of TBS were added to each well of the MaxiSorp® plates immediately, followed by the transfer of 70 μl of cell-free supernatant per sample. Additionally, 100 μl of recombinant human TNFα protein (provided as part of the DuoSet ELISA kit) diluted in TBS at defined concentrations were added to the plate as standard references. Thereafter, 100 μl per well of biotinylated goat anti-human TNFα detection antibody (provided as part of the DuoSet ELISA kit) at 50 ng/ml TBS were added and, protected from direct light, plates were incubated at room temperature for 2 hours. After 4 times washing with 350 μl per well of TBS-T (Carl Roth, Germany) on a 96-head plate washer (Tecan Group, Switzerland) and careful removal of all buffer traces after the fourth cycle, 100 μl of streptavidin-AP (R&D Systems, USA) diluted 1:10,000 in TBS were added to each well and, again protected from direct light, plates were incubated at room temperature for 30 minutes. Following another round of 4 times washing with 350 μl per well of TBS-T (Carl Roth, Germany) on a 96-head plate washer (Tecan Group, Switzerland) and careful removal of all buffer traces after the fourth cycle, 100 μl of AttoPhos substrate solution (Promega, USA) was added for incubation in the dark at room temperature for 1 hour. Using an infinite M1000 PRO (Tecan Group, Switzerland) microplate reader, the fluorescence of each well was collected at an excitation wavelength of 435 nm and an emission wavelength of 555 nm.

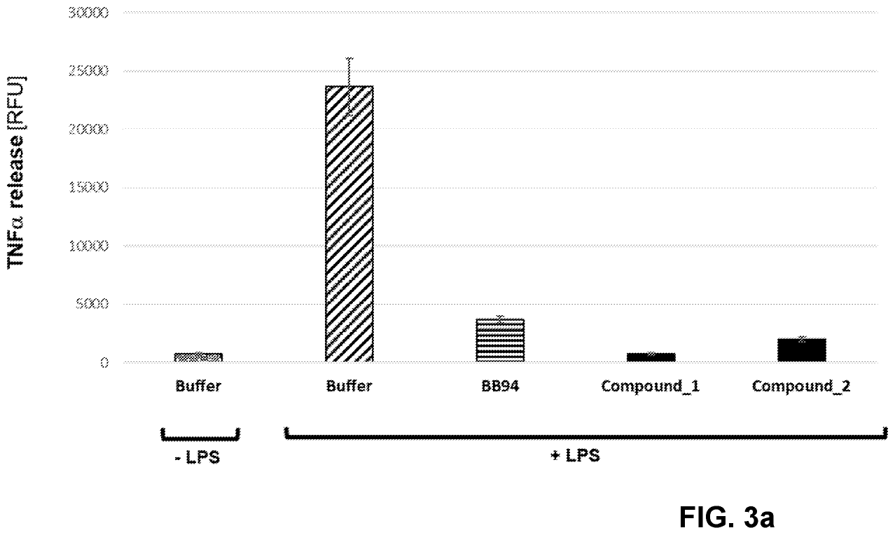

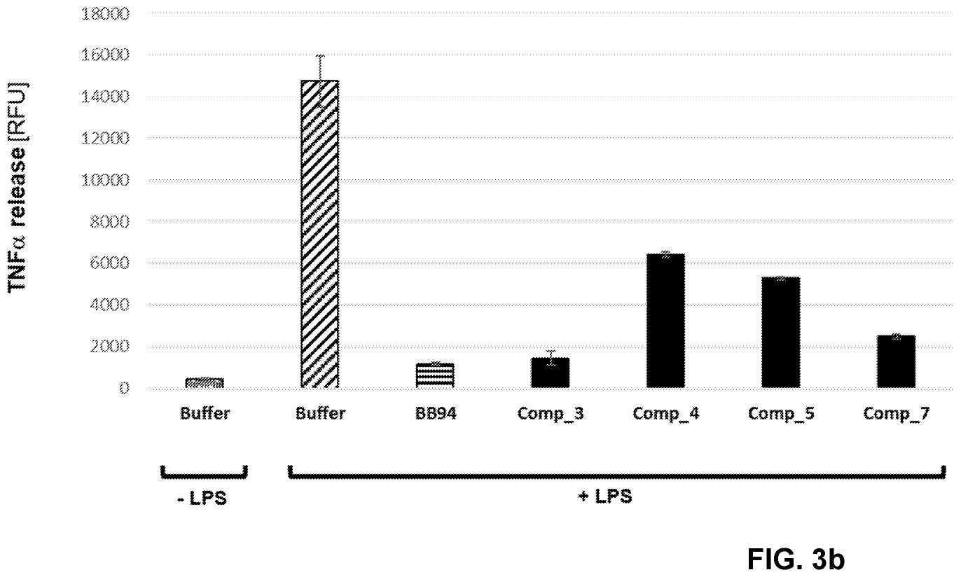

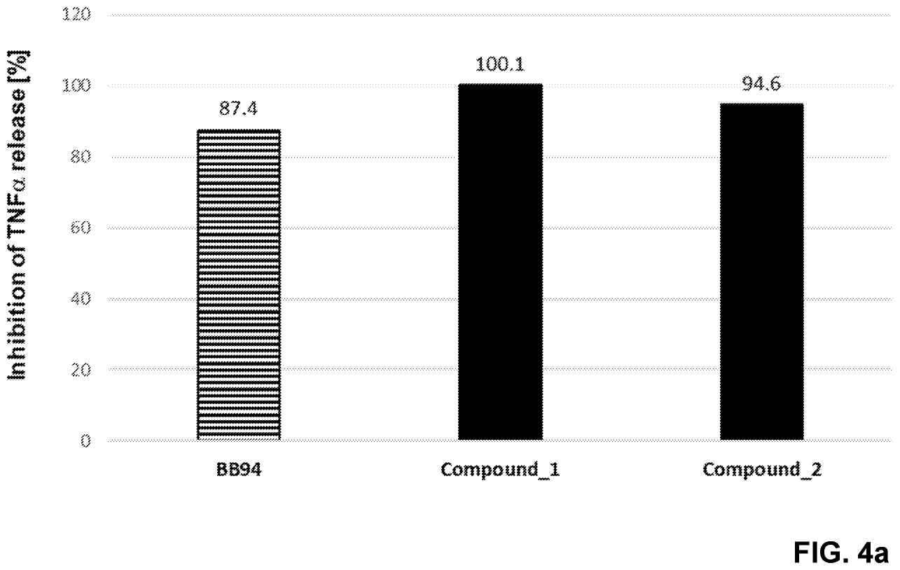

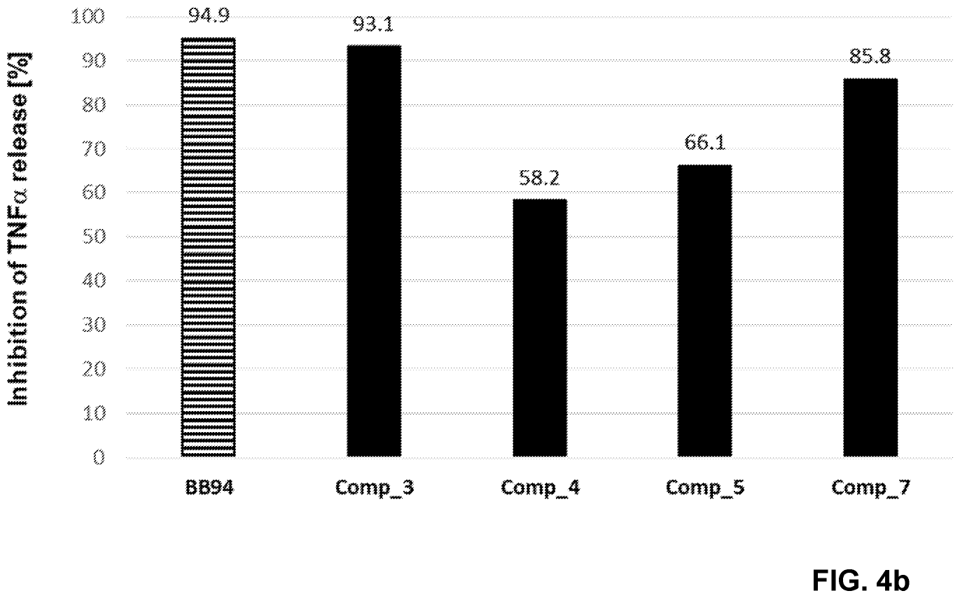

FIGS. 3a and 4a show representative results of this experiment demonstrating the effects of test articles on LPS-induced release of TNFα from THP-1 cells in absolute numbers (FIG. 3a) and percent inhibition (FIG. 4a). While Batimastat (BB94) as a small molecule inhibitor of metalloproteinases serves as positive control and results in 87.4% inhibition of LPS-induced release of TNFα, an equal concentration of either Compound 1 or Compound 2 inhibits LPS-induced release of TNFα from THP-1 cells by 100.1% and 94.6%, respectively.

FIGS. 3b and 4b show representative results of this experiment demonstrating the effects of test articles on LPS-induced release of TNFα from THP-1 cells in absolute numbers (FIG. 3b) and percent inhibition (FIG. 4b). While Batimastat (BB94) as a small molecule inhibitor of metalloproteinases serves as positive control and results in 94.9% inhibition of LPS-induced release of TNFα, an equal concentration of any of Compounds 3, 4, 5 or 7 inhibits LPS-induced release of TNFα from THP-1 cells by 93.1%, 58.2%, 66.1%, and 85.8%, respectively.

The delta between Buffer stimulated and Buffer unstimulated was defined 100% inhibition.

### Example 13: Analysis of Inhibitory Effects of the Compounds of the Invention on LPS-Induced TNFα Shedding in Primary Human Material from Healthy Donor In Vitro

In the following study, ELISA-based TNFα release assay was performed to verify the inhibitory effects of Compounds 1, 2, 3, 4, 5, and 7 on LPS-induced release of endogenous TNFα from primary human material obtained from healthy donor using peripheral blood mononuclear cells (PBMCs). The ELISA-based TNFα release assay that was used in this example is described below.

In brief, on day 1, Nunc black MaxiSorp® 96-well plates (Thermo Fisher Scientific, USA) were coated overnight with 100 μl per well of mouse anti-human TNFα capture antibody (provided as part of the DuoSet ELISA kit) at 4 μg/ml TBS at 4° C. On day 2, the capture antibody solution was removed and MaxiSorp® plates were blocked with 300 μl per well of TBS, 1% BSA at room temperature for 3 hours. Meanwhile, 20,000 PBMCs from healthy donor (SciRhom GmbH, Germany) in 80 μl of normal growth medium were seeded in each well of Greiner CELLSTAR V-bottom 96-well plates (Thermo Fisher Scientific, USA) and pre-incubated with 20 μl per well of standard growth medium supplemented with Batimastat (BB94, Abcam, UK) at 50 μM as positive control (for a final concentration of 10 μM in the resulting 100 μl sample volume), compounds of the invention at 50 μM (for a final concentration of 10 μM in the resulting 100 μl sample volume) and in case of Buffer stimulated and unstimulated controls with DMSO (Carl Roth, Germany) at 50 μM (for a final concentration of 10 μM in the resulting 100 μl sample volume) at 37° C., 5% CO₂ for 30 minutes. Subsequently, cells (except those for unstimulated controls) were stimulated with 20 μl per well of LPS (Sigma-Aldrich, USA) at 300 ng/ml growth medium for a final concentration of 50 ng/ml at 37° C., 5% CO₂ for 2 hours. Afterwards, the 96-well plates were centrifuged to pellet cells. In parallel, blocking buffer was removed from the MaxiSorp® plates and plates were washed 4 times with 350 μl per well of TBS-T (Carl Roth, Germany) on a 96-head plate washer (Tecan Group, Switzerland). To avoid drying-up, 30 μl of TBS were added to each well of the MaxiSorp® plates immediately, followed by the transfer of 70 μl of cell-free supernatant per sample. Additionally, 100 μl of recombinant human TNFα protein (provided as part of the DuoSet ELISA kit) diluted in TBS at defined concentrations were added to the plate as standard references. Thereafter, 100 μl per well of biotinylated goat anti-human TNFα detection antibody (provided as part of the DuoSet ELISA kit) at 50 ng/ml TBS were added and, protected from direct light, plates were incubated at room temperature for 2 hours. After 4 times washing with 350 μl per well of TBS-T (Carl Roth, Germany) on a 96-head plate washer (Tecan Group, Switzerland) and careful removal of all buffer traces after the fourth cycle, 100 μl of streptavidin-AP (R&D Systems, USA) diluted 1:10,000 in TBS were added to each well and, again protected from direct light, plates were incubated at room temperature for 30 minutes. Following another round of 4 times washing with 350 μl per well of TBS-T (Carl Roth, Germany) on a 96-head plate washer (Tecan Group, Switzerland) and careful removal of all buffer traces after the fourth cycle, 100 μl of AttoPhos substrate solution (Promega, USA) was added for incubation in the dark at room temperature for 1 hour. Using an infinite M1000 PRO (Tecan Group, Switzerland) microplate reader, the fluorescence of each well was collected at an excitation wavelength of 435 nm and an emission wavelength of 555 nm.

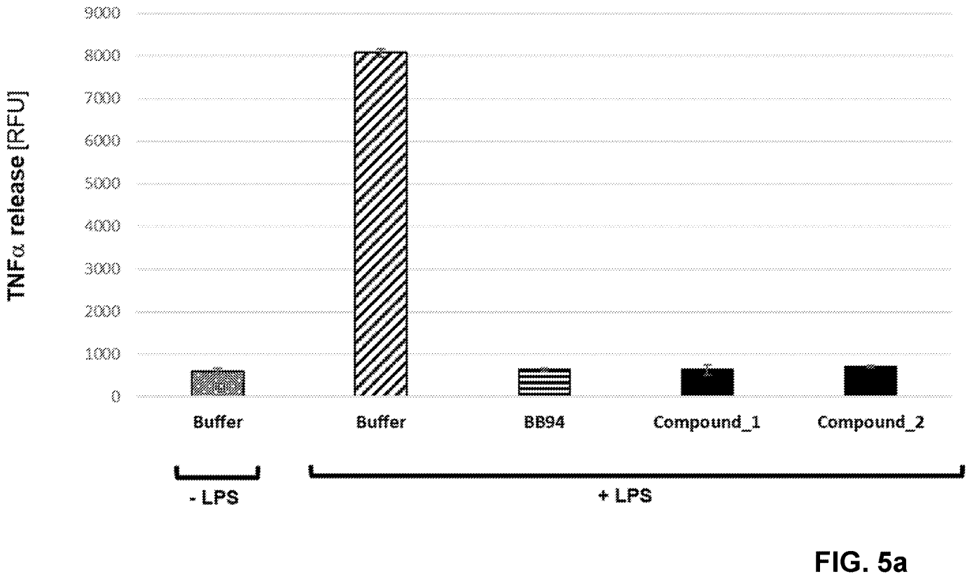

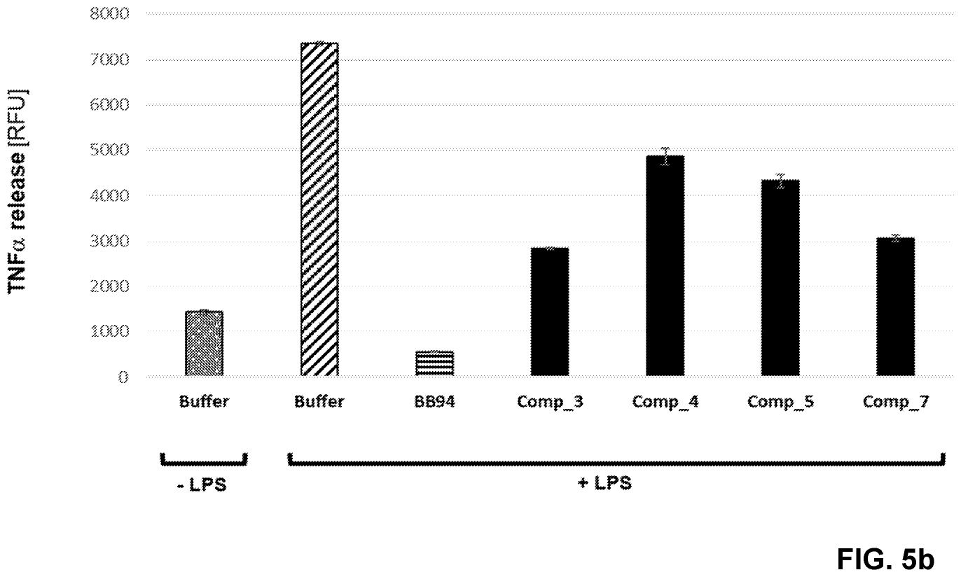

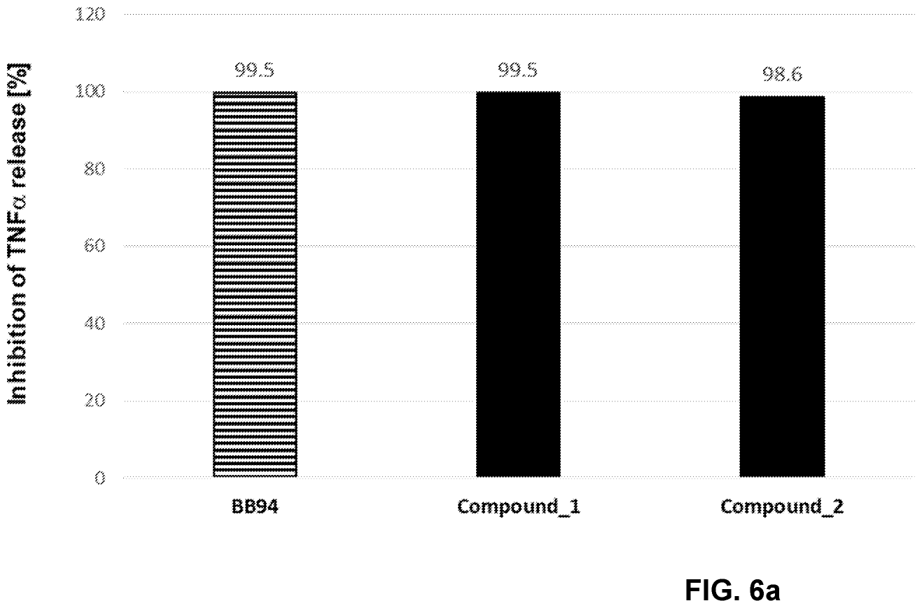

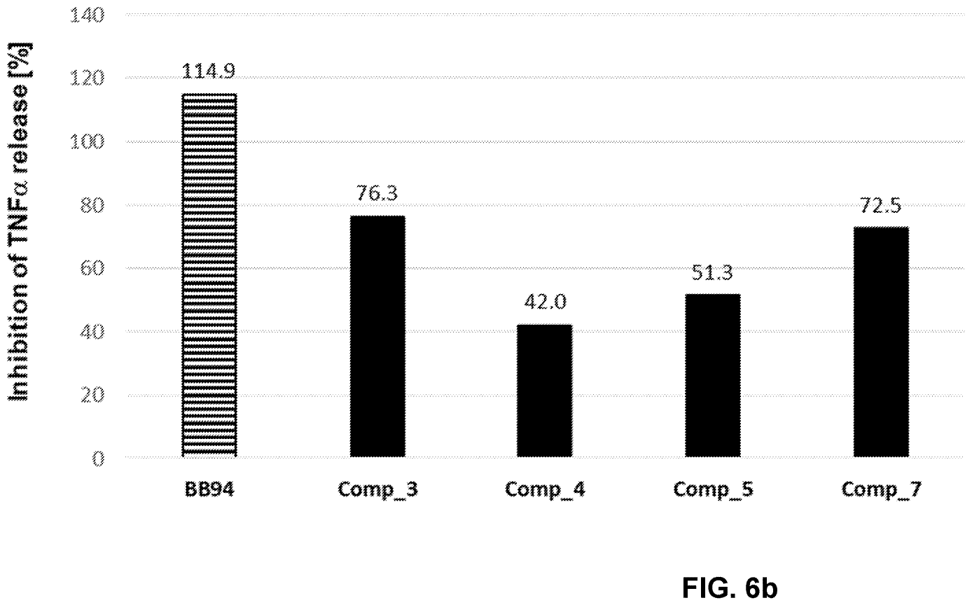

FIGS. 5a and 6a show representative results of this experiment demonstrating the effects of test articles on LPS-induced release of TNFα from human peripheral blood mononuclear cells (PBMCs) in absolute numbers (FIG. 5a) and percent inhibition (FIG. 6a). While Batimastat (BB94) as a small molecule inhibitor of serves as positive control and results in 99.5% inhibition of LPS-induced release of TNFα, an equal concentration of either Compound 1 or Compound 2 inhibits LPS-induced release of TNFα from human peripheral blood mononuclear cells (PBMCs) by 99.5% and 98.6%, respectively.

FIGS. 5b and 6b show representative results of this experiment demonstrating the effects of test articles on LPS-induced release of TNFα from human peripheral blood mononuclear cells (PBMCs) in absolute numbers (FIG. 5b) and percent inhibition (FIG. 6b). While Batimastat (BB94) as a small molecule inhibitor of serves as positive control and results in 114.9% inhibition of LPS-induced release of TNFα, an equal concentration of any of Compounds 3, 4, 5, or 7 inhibits LPS-induced release of TNFα from human peripheral blood mononuclear cells (PBMCs) by 76.3%, 42.0%, 51.3%, and 72.5% respectively.

The delta between Buffer stimulated and Buffer unstimulated was defined 100% inhibition.

### Example 14: Analysis of Inhibitory Effects of the Compounds of the Invention on PMA-Induced Interleukin 6 Receptor (IL-6R) Shedding in Primary Human Material from Healthy Donor In Vitro

In the following study, ELISA-based IL-6R release assays were performed to analyze the inhibitory effects of Compounds 1, 2, 3, 4, 5, and 7 on PMA-induced release of endogenous IL-6R from primary human material obtained from healthy donor using peripheral blood mononuclear cells (PBMCs). The ELISA-based IL-6R release assay that was used in this example is described below.

In brief, on day 1, Nunc black MaxiSorp® 96-well plates (Thermo Fisher Scientific, USA) were coated overnight with 100 pd per well of mouse anti-human IL-6R capture antibody (provided as part of the DuoSet ELISA kit) at 2 μg/ml TBS at 4° C.

40,000 PBMCs from healthy donor (SciRhom GmbH, Germany) in 80 μl of normal growth medium were seeded in each well of Greiner CELLSTAR V-bottom 96-well plates (Greiner Bio-One, Germany) and pre-incubated with 20 μl per well of standard growth medium supplemented with Batimastat (BB94, Abcam, UK) at 50 μM as positive control (for a final concentration of 10 μM in the resulting 100 μl sample volume), compounds of the invention at 50 μM (for a final concentration of 10 μM in the resulting 100 μl sample volume) and in case of Buffer stimulated and unstimulated controls with DMSO (Carl Roth, Germany) at 50 μM (for a final concentration of 10 μM in the resulting 100 μl sample volume) at 37° C., 5% CO₂ for 30 minutes. Subsequently, cells (except those for unstimulated controls) were stimulated with 20 μl per well of PMA (Sigma-Aldrich, USA) at 150 ng/ml in growth medium for a final concentration of 25 ng/ml at 37° C., 5% CO₂ for 22 hours.

On day 2, the capture antibody solution was removed and MaxiSorp® plates were blocked with 300 μl per well of TBS, 1% BSA at room temperature for 2-3 hours. Meanwhile, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human IL-6R protein (provided as part of the DuoSet ELISA kit) diluted in TBS at defined concentrations were added to the plate as standard references. Plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl biotinylated goat anti-human IL-6R detection antibody (provided as part of the DuoSet ELISA kit) at 100 ng/ml TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

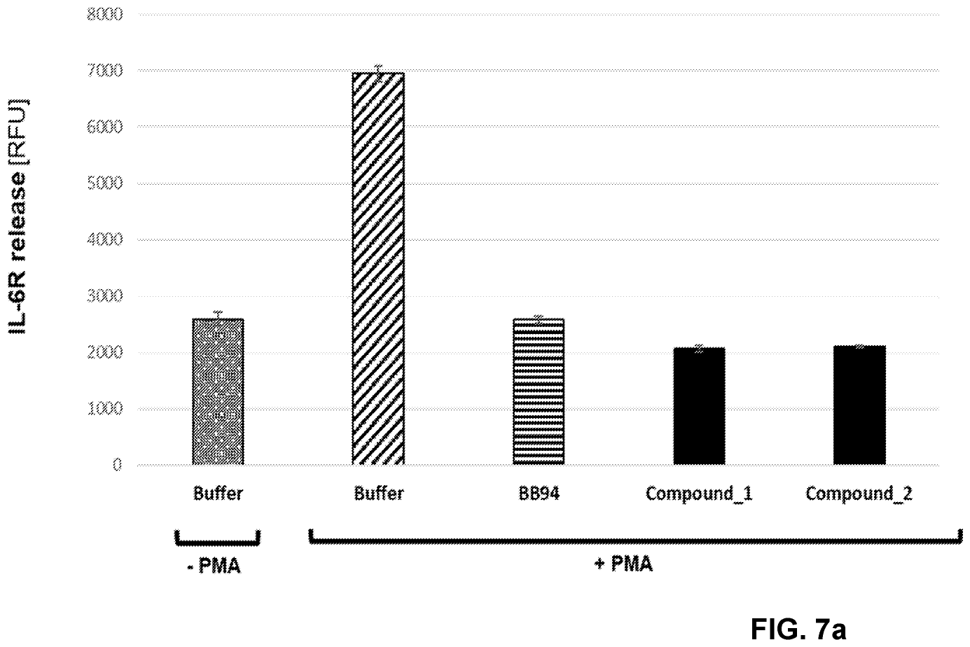

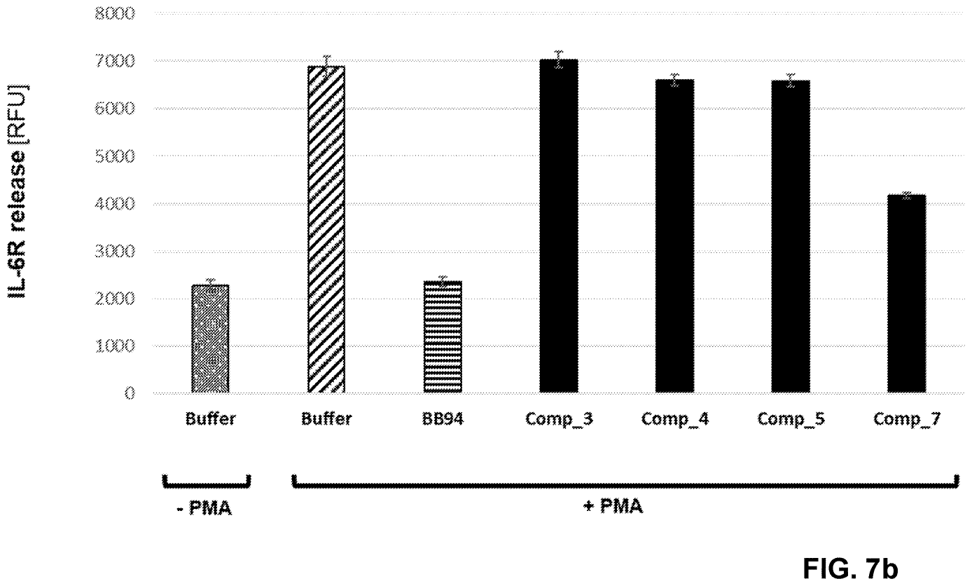

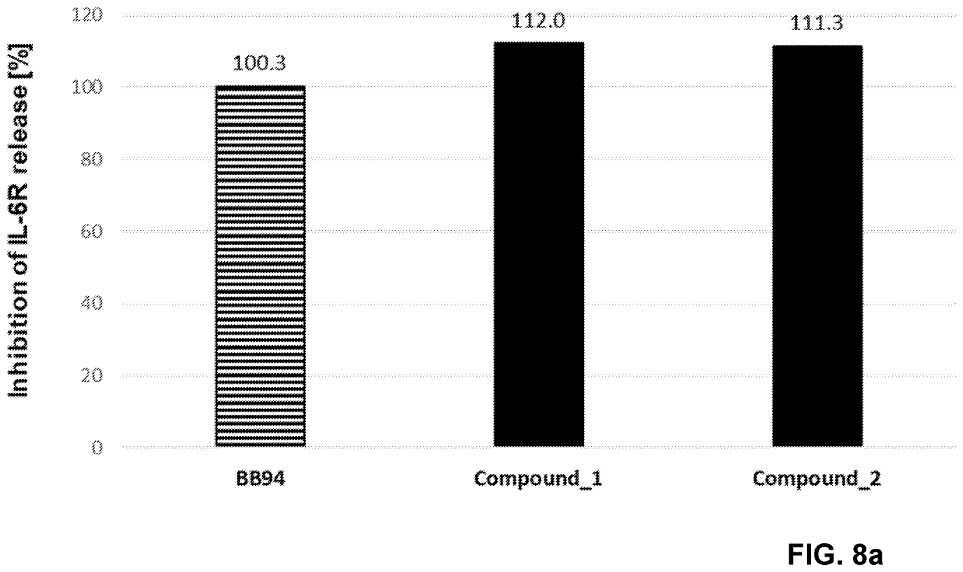

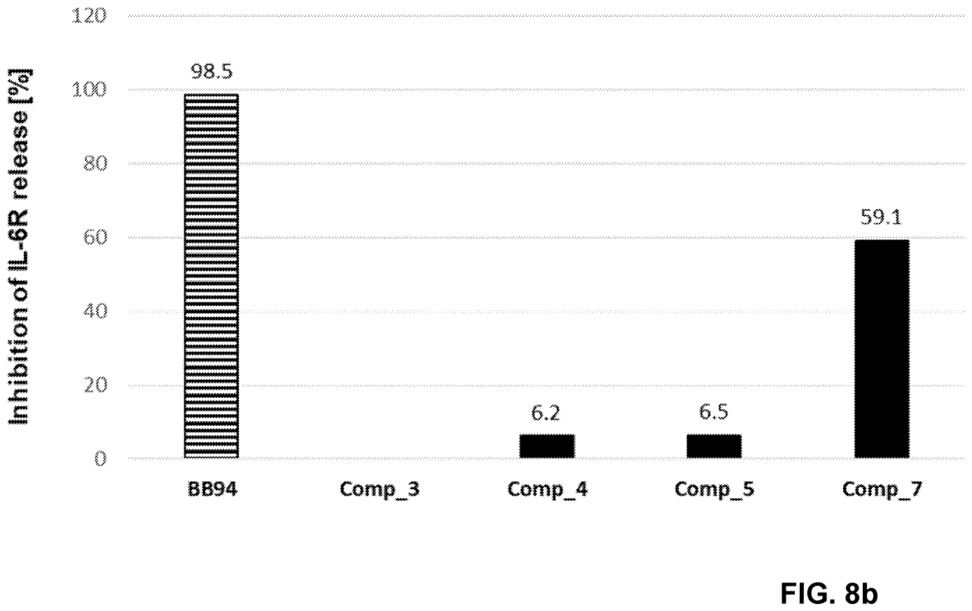

FIGS. 7a and 8a show representative results of this experiment demonstrating the effects of test articles on PMA-induced release of IL-6R from PBMCs from healthy donor in absolute numbers (FIG. 7a) and percent inhibition (FIG. 8a). While Batimastat (BB94) as a small molecule inhibitor of metalloproteinases serves as positive control and results in 100.3% inhibition of PMA-induced release of IL-6R, an equal concentration of either Compound 1 or Compound 2 inhibits PMA-induced release of IL-6R from PBMCs from healthy donor by 112.0% and 111.3%, respectively.

FIGS. 7b and 8b show representative results of this experiment demonstrating the effects of test articles on PMA-induced release of IL-6R from PBMCs from healthy donor in absolute numbers (FIG. 7b) and percent inhibition (FIG. 8b). While Batimastat (BB94) as a small molecule inhibitor of metalloproteinases serves as positive control and results in 98.5% inhibition of PMA-induced release of IL-6R, an equal concentration of any of Compounds 3, 4, 5, and 7 inhibits PMA-induced release of IL-6R from PBMCs from healthy donor by 0%, 6.2%, 6.5%, and 59.1%, respectively.

The delta between Buffer stimulated and Buffer unstimulated was defined 100% inhibition.

### Example 15: Analysis of Inhibitory Effects of the Compounds of the Invention on PMA-Induced HB-EGF Shedding in Human THP-1 Macrophage Cells In Vitro

In the following study, ELISA-based HB-EGF release assays were performed to analyze the inhibitory effects of Compounds 1, 2, 3, 4, 5, and 7 on PMA-induced release of endogenous HB-EGF from human THP-1 macrophage cells. The ELISA-based HB-EGF release assay that was used in this example is described below.

In brief, on day 1, Nunc black MaxiSorp® 96-well plates (Thermo Fisher Scientific, USA) were coated overnight with 100 μl per well of mouse anti-human HB-EGF capture antibody (provided as part of the DuoSet ELISA kit) at 2 μg/ml TBS at 4° C.

40,000 THP-1 cells (American Type Culture Collection, USA) cells in 80 μl of normal growth medium were seeded in each well of Greiner CELLSTAR V-bottom 96-well plates (Greiner Bio-One, Germany) and pre-incubated with 20 μl per well of standard growth medium supplemented with Batimastat (BB94, Abcam, UK) at 50 μM as positive control (for a final concentration of 10 μM in the resulting 100 μl sample volume), compounds of the invention at 50 μM (for a final concentration of 10 μM in the resulting 100 μl sample volume) and in case of Buffer stimulated and unstimulated controls with DMSO (Carl Roth, Germany) at 50 μM (for a final concentration of 10 μM in the resulting 100 μl sample volume) at 37° C., 5% CO₂ for 30 minutes. Subsequently, cells (except those for unstimulated controls) were stimulated with 20 μl per well of PMA (Sigma-Aldrich, USA) at 150 ng/ml in growth medium for a final concentration of 25 ng/ml at 37° C., 5% CO₂ for 23 hours.

On day 2, the capture antibody solution was removed and MaxiSorp® plates were blocked with 300 μl per well of TBS, 1% BSA at room temperature for 1-2 hours. Meanwhile, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human HB-EGF protein (provided as part of the DuoSet ELISA kit) diluted in TBS at defined concentrations were added to the plate as standard references. Plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl biotinylated goat anti-human HB-EGF detection antibody (provided as part of the DuoSet ELISA kit) at 50 ng/ml TBS were added per well and, protected from direct light, plates were incubated at room temperature for 2 hours.

After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

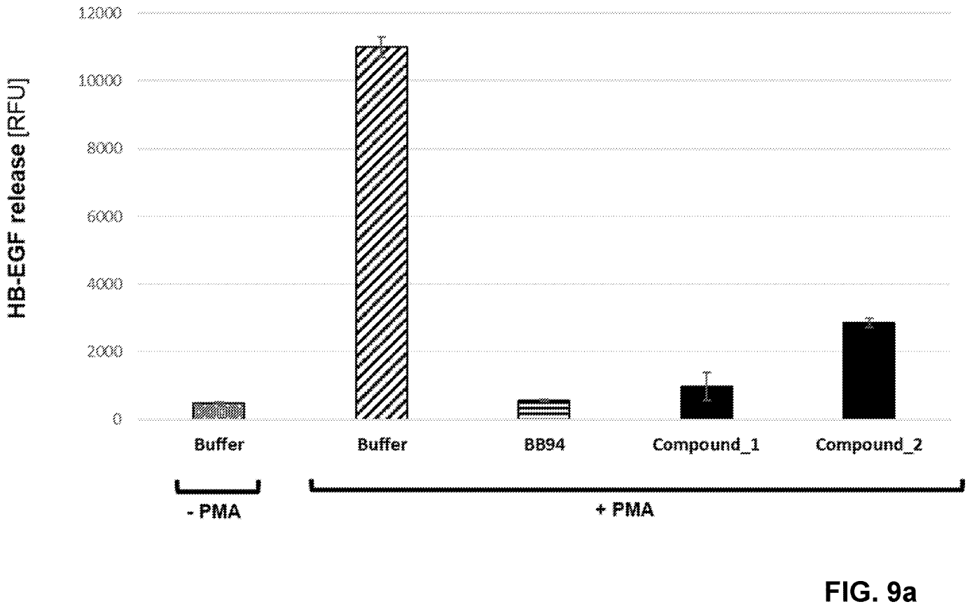

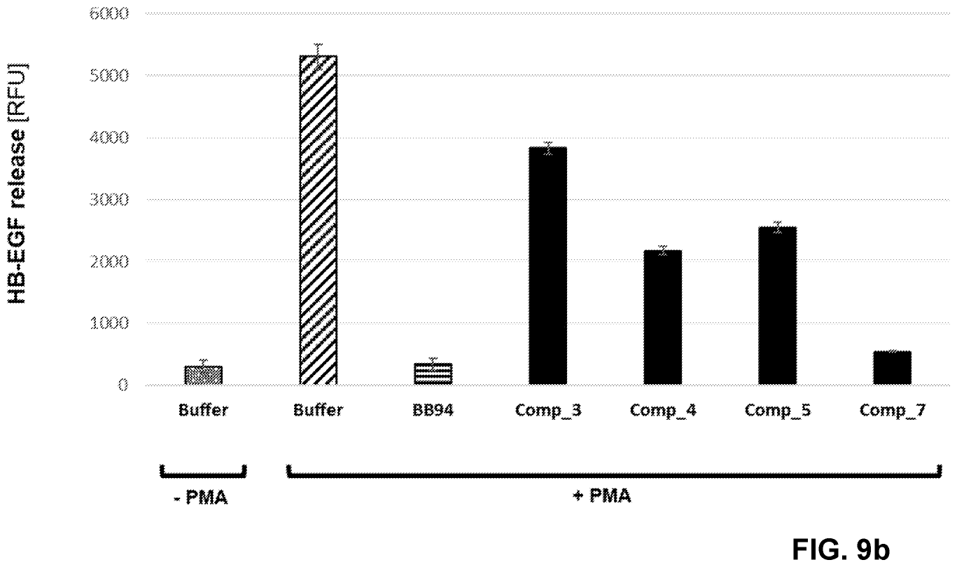

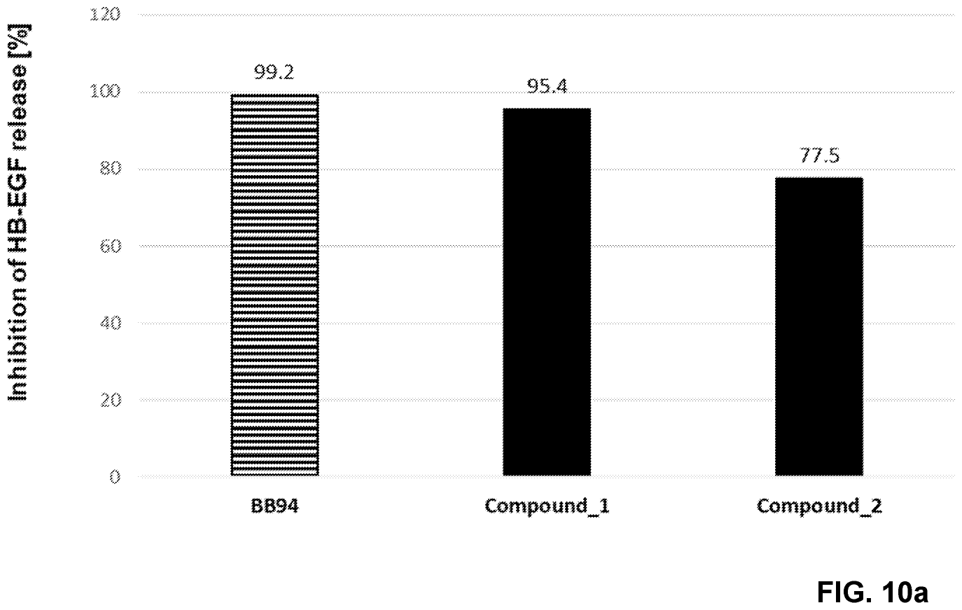

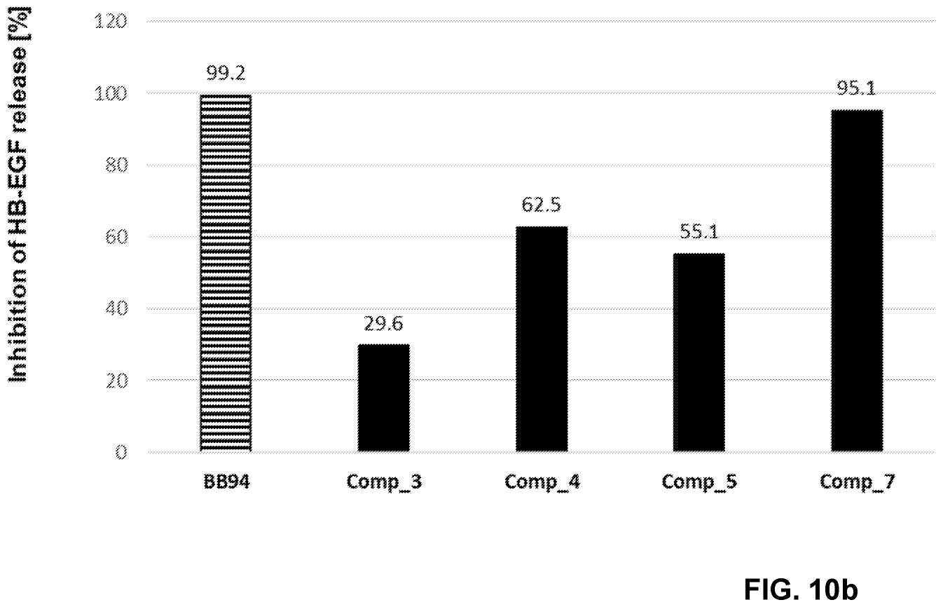

FIGS. 9a and 10a show representative results of this experiment demonstrating the effects of test articles on PMA-induced release of HB-EGF from THP-1 cells in absolute numbers (FIG. 9a) and percent inhibition (FIG. 10a). While Batimastat (BB94) as a small molecule inhibitor of metalloproteinases serves as positive control and results in 99.2% inhibition of PMA-induced release of HB-EGF, an equal concentration of either Compound 1 or Compound 2 inhibits PMA-induced release of HB-EGF from THP-1 cells by 95.4% and 77.5%, respectively.

FIGS. 9b and 10b show representative results of this experiment demonstrating the effects of test articles on PMA-induced release of HB-EGF from THP-1 cells in absolute numbers (FIG. 9b) and percent inhibition (FIG. 10b). While Batimastat (BB94) as a small molecule inhibitor of metalloproteinases serves as positive control and results in 99.2% inhibition of PMA-induced release of HB-EGF, an equal concentration of any of Compounds 3, 4, 5, and 7 inhibits PMA-induced release of HB-EGF from THP-1 cells by 29.6%, 62.5%, 55.1%, and 95.1%, respectively.

The delta between Buffer stimulated and Buffer unstimulated was defined 100% inhibition.

### Example 16: Analysis of Inhibitory Effects of the Compounds of the Invention on PMA-Induced HB-EGF Shedding in Primary Human Material from Healthy Donor In Vitro

In the following study, ELISA-based HB-EGF release assays were performed to analyze the inhibitory effects of Compounds 1, 2, 3, 4, 5, and 7 on PMA-induced release of endogenous HB-EGF from primary human material obtained from healthy donor using peripheral blood mononuclear cells (PBMCs). The ELISA-based HB-EGF release assay that was used in this example is described below.

In brief, on day 1, Nunc black MaxiSorp® 96-well plates (Thermo Fisher Scientific, USA) were coated overnight with 100 μl per well of mouse anti-human HB-EGF capture antibody (provided as part of the DuoSet ELISA kit) at 2 μg/ml TBS at 4° C.

80,000 PBMCs from healthy donor (SciRhom GmbH, Germany) in 80 μl of normal growth medium were seeded in each well of Greiner CELLSTAR V-bottom 96-well plates (Greiner Bio-One, Germany) and pre-incubated with 20 d per well of standard growth medium supplemented with Batimastat (BB94, Abcam, UK) at 50 μM as positive control (for a final concentration of 10 μM in the resulting 100 μl sample volume), compounds of the invention at 50 μM (for a final concentration of 10 μM in the resulting 100 μl sample volume) and in case of Buffer stimulated and unstimulated controls with DMSO (Carl Roth, Germany) at 50 μM (for a final concentration of 10 μM in the resulting 100 μl sample volume) at 37° C., 5% CO₂ for 30 minutes. Subsequently, cells (except those for unstimulated controls) were stimulated with 20 μl per well of PMA (Sigma-Aldrich, USA) at 150 ng/ml in growth medium for a final concentration of 25 ng/ml at 37° C., 5% CO₂ for 22 hours.

On day 2, the capture antibody solution was removed and MaxiSorp® plates were blocked with 300 μl per well of TBS, 1% BSA at room temperature for 1-2 hours. Meanwhile, the 96-well plates were centrifuged to pellet cells. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 30 μl TBS were added to each well of the MaxiSorp® plates immediately, followed by the transfer of 70 μl cell-free supernatant per sample. Additionally, 100 μl recombinant human HB-EGF protein (provided as part of the DuoSet ELISA kit) diluted in TBS at defined concentrations were added to the plate as standard references. Plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl biotinylated goat anti-human HB-EGF detection antibody (provided as part of the DuoSet ELISA kit) at 50 ng/ml TBS were added per well and, protected from direct light, plates were incubated at room temperature for 2 hours.

After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

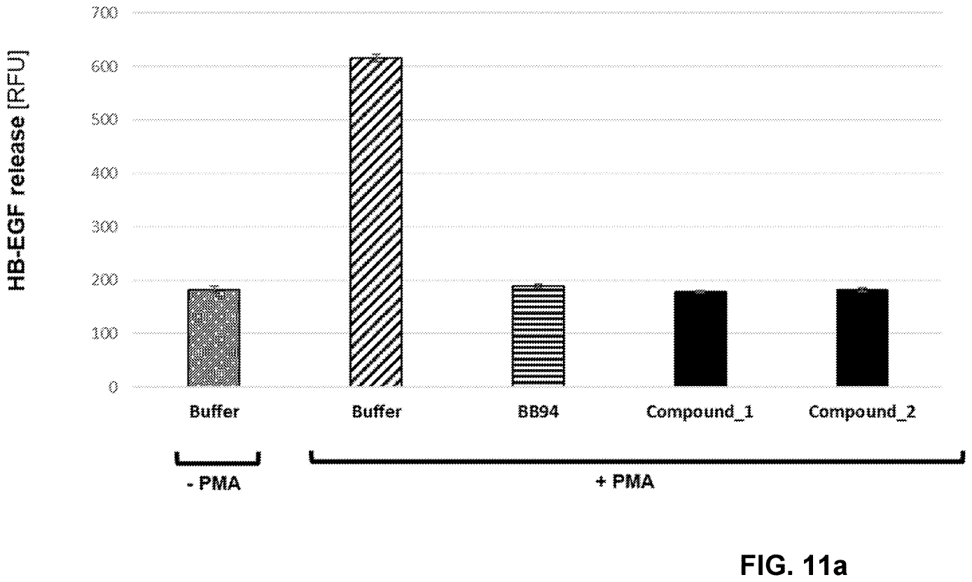

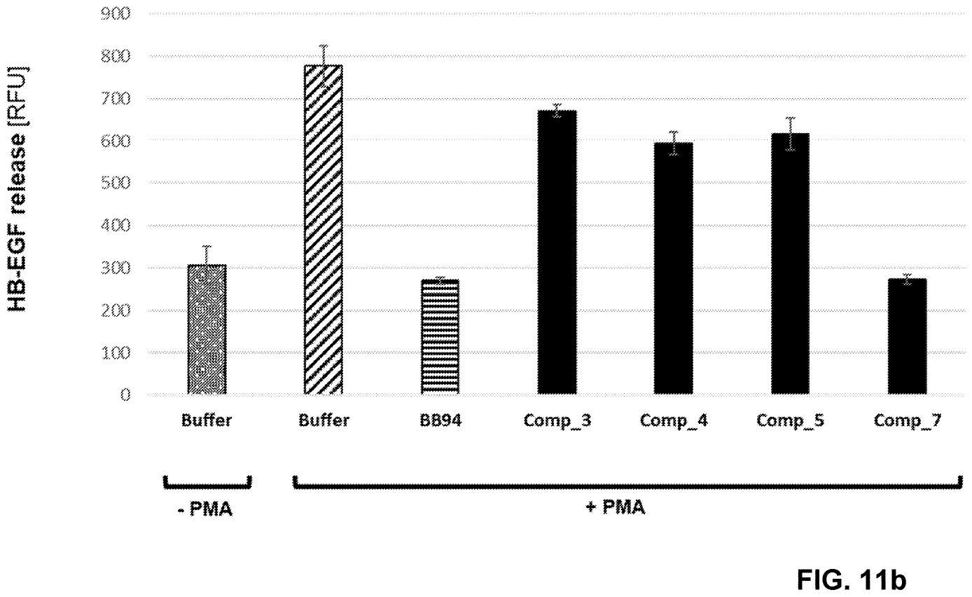

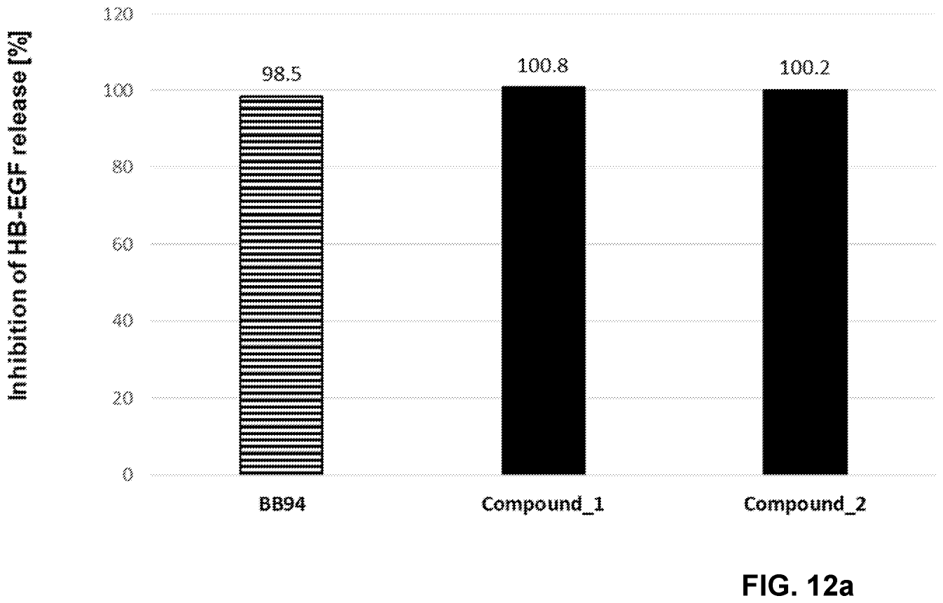

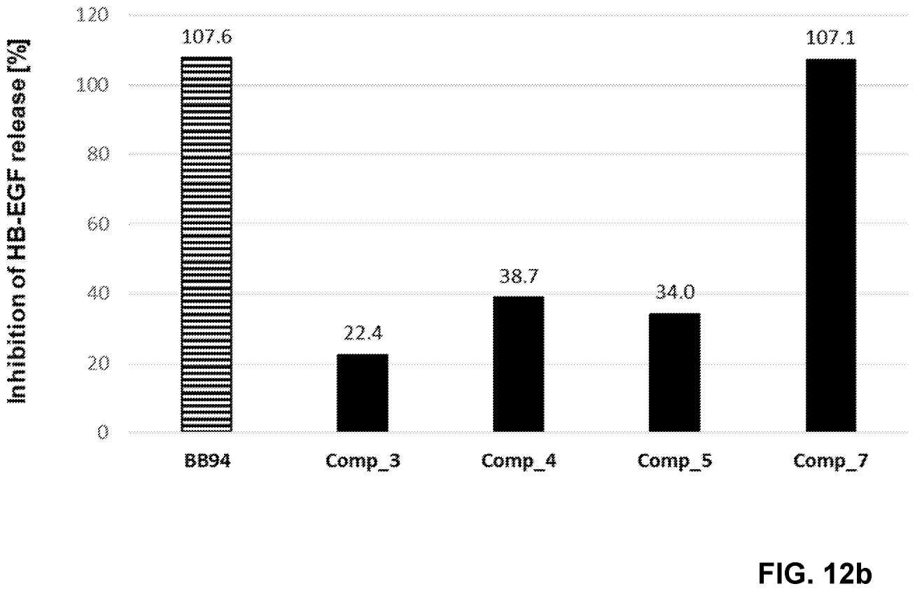

FIGS. 11a and 12a show representative results of this experiment demonstrating the effects of test articles on PMA-induced release of HB-EGF from PBMCs from healthy donor in absolute numbers (FIG. 11a) and percent inhibition (FIG. 12a). While Batimastat (BB94) as a small molecule inhibitor of metalloproteinases serves as positive control and results in 98.5% inhibition of PMA-induced release of HB-EGF, an equal concentration of either Compound 1 or Compound 2 inhibits PMA-induced release of HB-EGF from PBMCs from healthy donor by 100.8% and 100.2%, respectively.

FIGS. 11b and 12b show representative results of this experiment demonstrating the effects of test articles on PMA-induced release of HB-EGF from PBMCs from healthy donor in absolute numbers (FIG. 11b) and percent inhibition (FIG. 12b). While Batimastat (BB94) as a small molecule inhibitor of metalloproteinases serves as positive control and results in 107.6% inhibition of PMA-induced release of HB-EGF, an equal concentration of any of Compounds 3, 4, 5, and 7 inhibits PMA-induced release of HB-EGF from PBMCs from healthy donor by 22.4%, 38.7%, 34.0%, and 107.1%, respectively.

The delta between Buffer stimulated and Buffer unstimulated was defined 100% inhibition.

### Example 17: Analysis of Inhibitory Effects of the Compounds of the Invention on PMA-Induced Transforming Growth Factor Alpha (TGFα) Shedding in Human PC3 Prostate Cancer Cells In Vitro

In the following study, ELISA-based TGFα release assays were performed to analyze the inhibitory effects of Compounds 1, 2, 3, 4, 5, and 7 on PMA-induced release of endogenous TGFα, from human PC3 prostate cancer cells. The ELISA-based TGFα release assay that was used in this example is described below.

In brief, on day 1, Nunc black MaxiSorp® 96-well plates (Thermo Fisher Scientific, USA) were coated overnight with 100 μl per well of goat anti-human TGFα capture antibody (provided as part of the DuoSet ELISA kit) at 0.4 μg/ml TBS at 4° C.

75,000 PC3 (European Collection of Authenticated Cell Cultures, UK) cells in 100 μl of normal growth medium were seeded in each well of F-bottom 96-well cell culture plates (Corning, USA) and incubated at 37° C., 5% CO₂ overnight. On day 2, the capture antibody solution was removed and MaxiSorp® plates were blocked with 300 μl per well of TBS, 1% BSA at room temperature for 5 hours. Meanwhile, cells were washed twice with PBS and pre-incubated in 80 μl of OptiMEM medium with 20 μl per well of OptiMEM medium supplemented with Batimastat (BB94, Abcam, UK) at 50 μM as positive control (for a final concentration of 10 μM in the resulting 100 μl sample volume), compounds of the invention at 50 μM (for a final concentration of 10 μM in the resulting 100 μl sample volume) and in case of Buffer stimulated and unstimulated controls with DMSO (Carl Roth, Germany) at 50 μM (for a final concentration of 10 μM in the resulting 100 μl sample volume) at 37° C., 5% CO₂ for 30 minutes. Subsequently, cells (except those for unstimulated controls) were stimulated with 20 μl per well of PMA (Sigma-Aldrich, USA) at 150 ng/ml in OptiMEM for a final concentration of 25 ng/ml at 37° C., 5% CO₂ for 2 hours. In parallel, blocking buffer was removed from the MaxiSorp® plates and plates were washed 4 times with 350 μl TBS-T (Carl Roth, Germany) per well on a 96-head plate washer (Tecan Group, Switzerland). To avoid drying-up, 20 μl TBS were added to each well of the MaxiSorp® plates immediately, followed by the transfer of 80 μl cell-free supernatant per sample. Additionally, 100 μl recombinant human TGFα protein (provided as part of the DuoSet ELISA kit) diluted in TBS at defined concentrations were added to the plate as standard references. Thereafter, 100 μl biotinylated goat anti-human TGFα detection antibody (provided as part of the DuoSet ELISA kit) at 37.5 ng/ml in TBS were added per well and, protected from direct light, plates were incubated at room temperature for 2 hours. After 4 times washing with 350 μl TBS-T (Carl Roth, Germany) per well on a 96-head plate washer (Tecan Group, Switzerland) and careful removal of all buffer traces after the fourth cycle, 100 μl streptavidin-AP (R&D Systems, USA) diluted 1:10,000 in TBS were added to each well and, again protected from direct light, plates were incubated at room temperature for 30 minutes. Following another round of 4 times washing with 350 μl TBS-T (Carl Roth, Germany) per well on a 96-head plate washer (Tecan Group, Switzerland) and careful removal of all buffer traces after the fourth cycle, 100 μl AttoPhos substrate solution (Promega, USA) per well was added for incubation in the dark at room temperature for 1 hour. Using an infinite M1000 (Tecan Group, Switzerland) microplate reader, the fluorescence of each well was collected at an excitation wavelength of 435 nm and an emission wavelength of 555 nm.

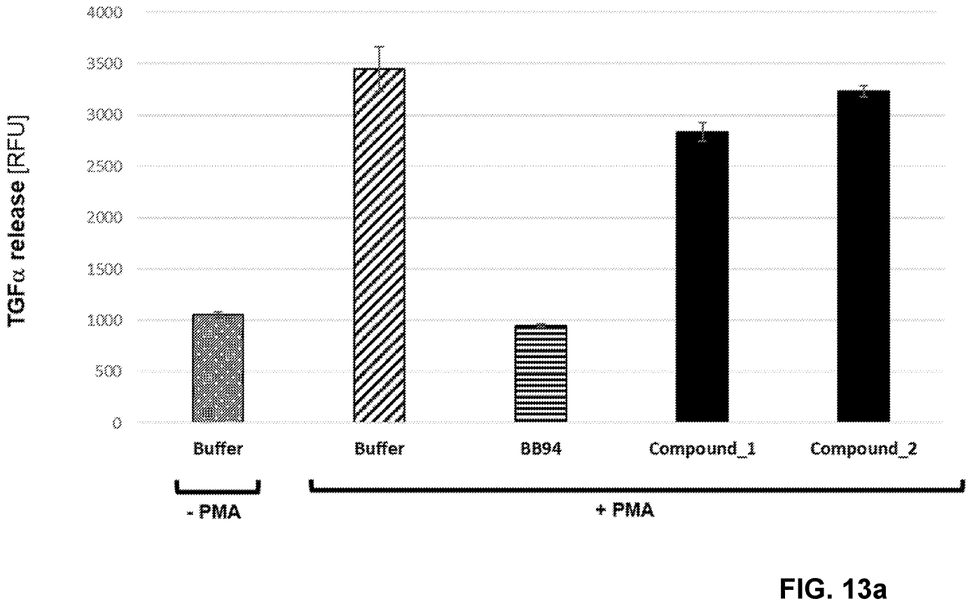

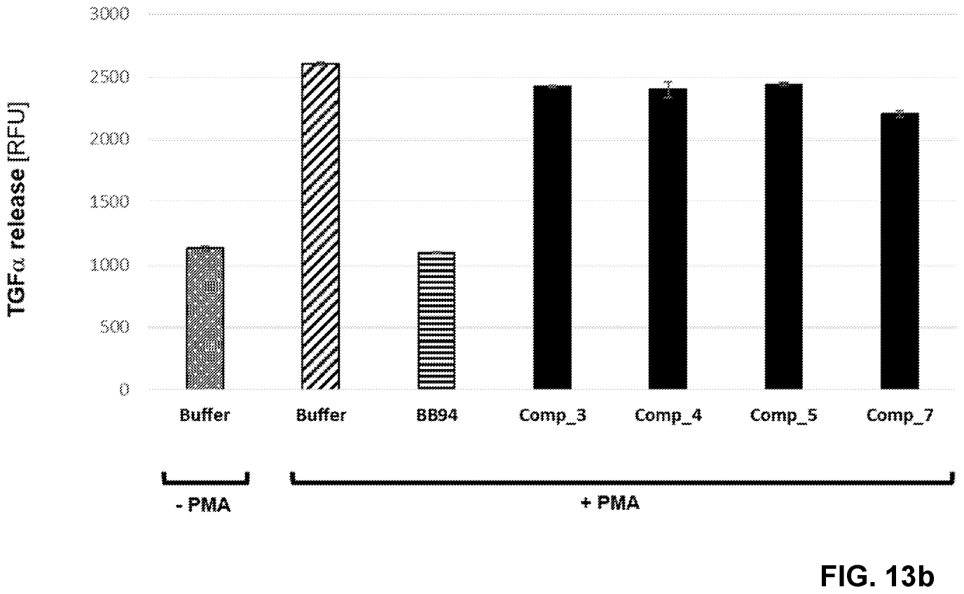

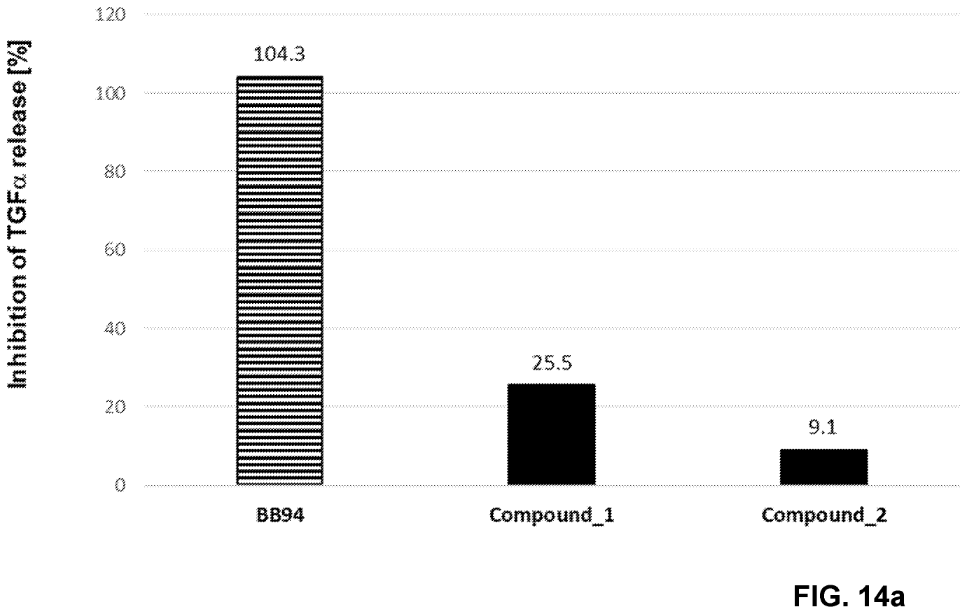

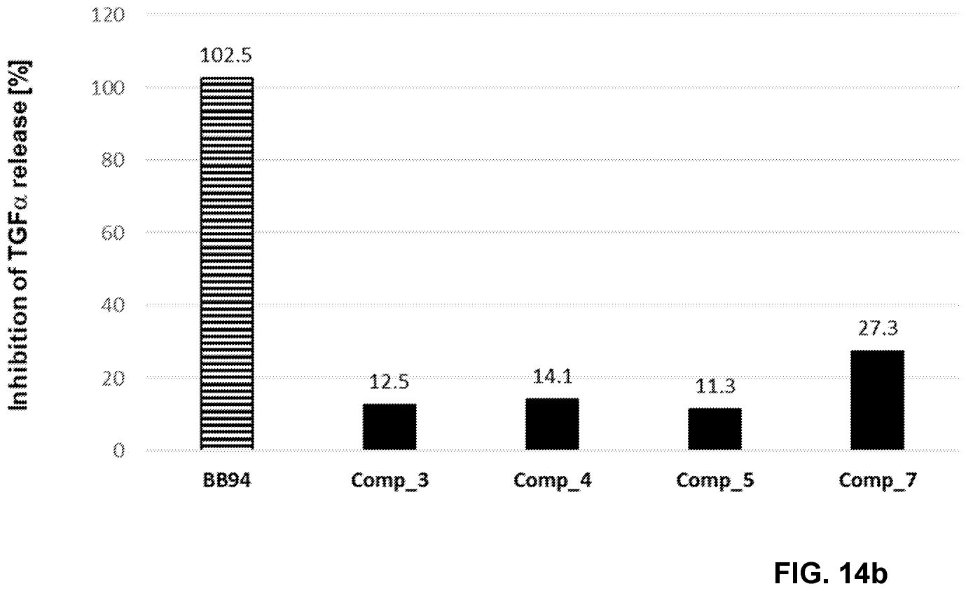

FIGS. 13a and 14a show representative results of this experiment demonstrating the effects of test articles on PMA-induced release of TGFα from PC3 cells in absolute numbers (FIG. 13a) and percent inhibition (FIG. 14a). While Batimastat (BB94) as a small molecule inhibitor of metalloproteinases serves as positive control and results in 104.3% inhibition of PMA-induced release of TGFα, only a very moderate effect on TGFα shedding was detected in the presence of an equal concentration of either Compound 1 or Compound 2, which inhibit PMA-induced release of TGFα from PC3 cells by 25.5% and 9.1%, respectively.

FIGS. 13b and 14b show representative results of this experiment demonstrating the effects of test articles on PMA-induced release of TGFα from PC3 cells in absolute numbers (FIG. 13b) and percent inhibition (FIG. 14b). While Batimastat (BB94) as a small molecule inhibitor of metalloproteinases serves as positive control and results in 102.5% inhibition of PMA-induced release of TGFα, only a very moderate effect on TGFα shedding was detected in the presence of an equal concentration of any of Compounds 3, 4, 5, and 7, which inhibit PMA-induced release of TGFα from PC3 cells by 12.5%, 14.1%, 11.3%, and 27.3%, respectively.

The delta between Buffer stimulated and Buffer unstimulated was defined 100% inhibition.

Various modifications of the invention, in addition to those described herein, will be apparent to those skilled in the art from the foregoing description. Such modifications are also intended to fall within the scope of the appended claims. Each reference, including without limitation all patent, patent applications, and publications, cited in the present application is incorporated herein by reference in its entirety.

1. A method of inhibiting iRhom2/ADAM17 activity, said method comprising administering to a patient a compound of Formula (I): 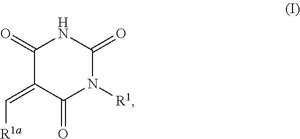
or a pharmaceutically acceptable salt thereof, wherein:
R¹ is C₆₋₁₀ aryl, 5-10 membered heteroaryl, 4-10 membered heterocycloalkyl, or C₃₋₁₀ cycloalkyl;
R^{1a} is C₆₋₁₀ aryl, 5-10 membered heteroaryl, 4-10 membered heterocycloalkyl, or C₃₋₁₀ cycloalkyl;
wherein each R¹ or R^{1a} is optionally substituted with 1, 2 or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄alkyl)), —C₀₋₄ alkyl(NHC(O)NH₂), —C₀₋₄ alkyl(NHC(O)NH(C₁₋₄alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

2. The method of claim 1, wherein R¹ is C₆₋₁₀ aryl optionally substituted with 1, 2 or 3 substituents selected from the group consisting of halo and C₁₋₆ alkyl.

3. The method of claim 1 or 2, wherein R¹ is

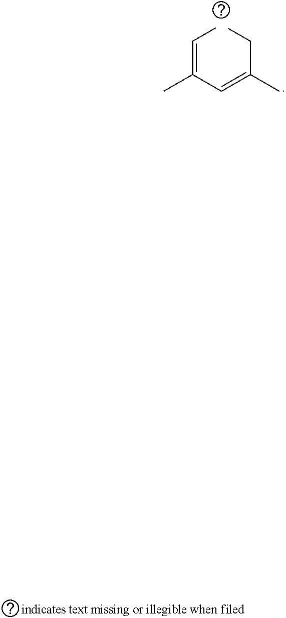

4. The method of any one of claims 1-3, wherein R^{1a} is a 5-10 membered heteroaryl optionally substituted with 1, 2 or 3 substituents selected from the group consisting of halo, C₁₋₆ alkyl, C₁₋₄ alkyl(NHC(O)C₁₋₄ alkyl), C₁₋₄ alkyl(C(O)C₁₋₄ alkyl), C₁₋₄ alkyl(C(O)NHC₁₋₄ alkyl), C₁₋₄ alkyl(OC(O)C₁₋₄ alkyl) and C₁₋₄ alkyl((O)COC₁₋₄ alkyl),

5. The method of any one of claims 1-4, wherein R^{1a} is indole optionally substituted with 1, 2 or 3 substituents selected from the group consisting of halo, C₁₋₆ alkyl, C₁₋₄ alkyl(NHC(O)C₁₋₄ alkyl), C₁₋₄ alkyl(C(O)C₁₋₄ alkyl), C₁₋₄ alkyl(C(O)NHC₁₋₄ alkyl), C₁₋₄ alkyl(OC(O)C₁₋₄ alkyl) and C₁₋₄ alkyl((O)COC₁₋₄ alkyl).

6. The method of any one of claims 1-5, wherein R^{1a} is

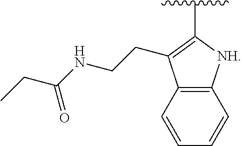 [SMILES: *c1[nH]c2ccccc2c1CCNC(=O)CC]

7. The method of any one of claims 1-6, wherein the compound of Formula (I) is: 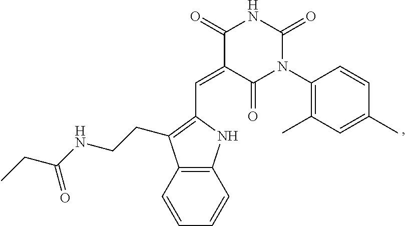 [SMILES: CCC(=O)NCCc1c(/C=C2/C(=O)NC(=O)N(c3ccc(C)cc3C)C2=O)[nH]c2ccccc12]
or a pharmaceutically acceptable salt thereof.

8. A method of inhibiting iRhom2/ADAM17 activity, said method comprising administering to a patient a compound of Formula (II): 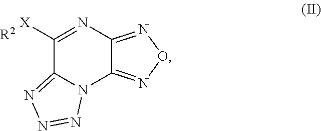
or a pharmaceutically acceptable salt thereof; wherein:
or C(C₁₋₄alkyl)₂;
R² is C₆₋₁₀ aryl, 5-10 membered heteroaryl, 4-10 membered heterocycloalkyl, or C₃₋₁₀ cycloalkyl;
wherein each R² is optionally substituted with 1, 2 or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

9. The method of claim 8, wherein X is NH.

10. The method of claim 8 or 9, wherein R² is C₆₋₁₀ aryl optionally substituted with 1, 2 or 3 substituents selected from the group consisting of halo, C₁₋₆ alkyl, —C(O)(C₁₋₄ alkyl), —C(O)NH₂, —C(O)NH(C₁₋₄ alkyl), —C(O)N(C₁₋₄ alkyl)₂, —C(O)O(C₁₋₄ alkyl), S(O)(C₁₋₄ alkyl), —S(O)NH₂, —S(O)NH(C₁₋₄ alkyl), —S(O)N(C₁₋₄ alkyl)₂, —S(O)₂(C₁₋₄ alkyl), —S(O)₂NH₂, —S(O)₂NH(C₁₋₄ alkyl), and —S(O)₂N(C₁₋₄ alkyl)₂.

11. The method of any one of claims 8-10, wherein R² is

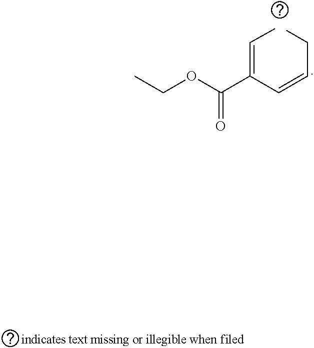

12. The method of any one of claims 8-11, wherein the compound of Formula (II) is: 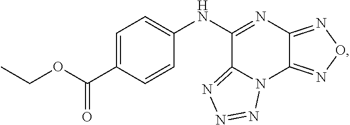 [SMILES: CCOC(=O)c1ccc(Nc2nc3nonc3n3nnnc23)cc1]
or a pharmaceutically acceptable salt thereof.

13. A method of inhibiting iRhom2/ADAM17 activity, said method comprising administering to a patient a compound of Formula (III): 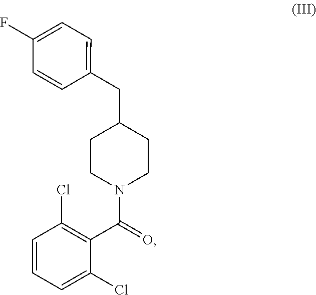 [SMILES: O=C(c1c(Cl)cccc1Cl)N1CCC(Cc2ccc(F)cc2)CC1]
or a pharmaceutically acceptable salt thereof.

14. A method of inhibiting iRhom2/ADAM17 activity, said method comprising administering to a patient a compound of Formula (IV): 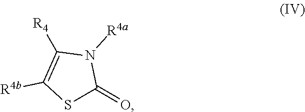
or a pharmaceutically acceptable salt thereof; wherein:
R⁴ is H, C₁₋₄ haloalkyl, or C₁₋₄ alkyl;
R^{4a} is —C₀₋₆ alkyl(C₆₋₁₀ aryl), —C₀₋₆ alkyl(5-10 membered heteroaryl), —C₀₋₆ alkyl(C₃₋₁₀cycloalkyl), or —C₀₋₆ alkyl(4-10 membered heterocycloalkyl);
R^{4b} is —C(O)—C₀₋₄alkyl(C₆₋₁₀ aryl), —C(O)—C₀₋₄alkyl(5-10 membered heteroaryl), —C(O)—C₀₋₄alkyl(C₃₋₁₀ cycloalkyl), —C(O)—C₀₋₄alkyl(4-10 membered heterocycloalkyl), —NHC(O)—C₀₋₄alkyl(C₆₋₁₀ aryl), —NHC(O)—C₀₋₄alkyl(5-10 membered heteroaryl), —NHC(O)—C₀₋₄alkyl(C₃₋₁₀—C(O)OC₀₋₄alkyl(5-10 membered heteroaryl), —C(O)OC₀₋₄alkyl(C₃₋₁₀ cycloalkyl), —C(O)OC₀₋₄alkyl(4-10 membered heterocycloalkyl), —C(O)NH—C₀₋₄alkyl(C₆₋₁₀ aryl), —C(O)NH—C₀₋₄alkyl(5-10 membered heteroaryl), —C(O)NH—C₀₋₄alkyl(C₃₋₁₀ cycloalkyl), —C(O)NH—C₀₋₄alkyl(4-10 membered heterocycloalkyl), —C(O)N(C₁₋₆ alkyl)(C₀₋₄alkyl-C₆₋₁₀ aryl), —C(O)N(C₁₋₆ alkyl)(C₀₋₄alkyl-(5-10 membered heteroaryl)), —C(O)N(C₁₋₆ alkyl)(C₀₋₄alkyl-C₃₋₁₀ cycloalkyl), or —C(O)N(C₁₋₆ alkyl)(C₀₋₄alkyl-(4-10 membered heterocycloalkyl));
wherein each R^{4a} or R^{4b} is optionally substituted with 1, 2, or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

15. The method of claim 14, wherein R⁴ is methyl.

16. The method of claim 14 or 15, wherein R^{4a} is —C₁₋₆ alkyl(C₆₋₁₀ aryl) optionally substituted with 1, 2, or 3 substituents selected from the group consisting of halo and —OC(O)(C₁₋₄ alkyl).
of

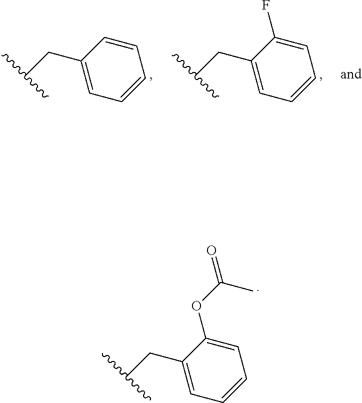 [SMILES: *Cc1ccccc1 ; *Cc1ccccc1F ; *Cc1ccccc1OC(C)=O]

18. The method of any one of claims 14-17, wherein R^{4b} is —C(O)NH—C₁₋₄alkyl(C₆₋₁₀ aryl), —C(O)NH—C₁₋₄alkyl(5-10 membered heteroaryl), —C(O)NH—C₁₋₄alkyl(C₃₋₁₀cycloalkyl), —C(O)NH—C₁₋₄alkyl(4-10 membered heterocycloalkyl), —C(O)N(C₁₋₆ alkyl)(C₁₋₄alkyl-C₆₋₁₀ aryl), —C(O)N(C₁-6 alkyl)(C₁₋₄alkyl-(5-10 membered heteroaryl)), —C(O)N(C₁₋₆ alkyl)(C₁₋₄alkyl-C₃₋₁₀ cycloalkyl), or —C(O)N(C₁₋₆ alkyl)(C₁₋₄alkyl-(4-10 membered heterocycloalkyl)).

19. The method of any one of claims 14-18, wherein R^{4b} is —C(O)NH—C₁₋₄alkyl(5-10 membered heteroaryl).

20. The method of any one of claims 14-19, wherein R^{4b} is selected from the group consisting of

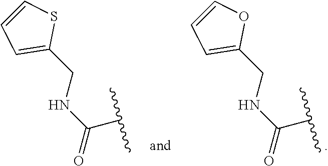 [SMILES: *C(=O)NCc1cccs1 ; *C(=O)NCc1ccco1]

21. The method of any one of claims 14-20, wherein the compound of Formula (IV) is: 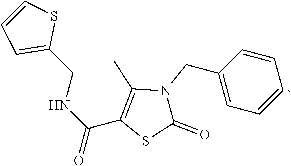 [SMILES: Cc1c(C(=O)NCc2cccs2)sc(=O)n1Cc1ccccc1]
or a pharmaceutically acceptable salt thereof.

22. The method of any one of claims 14-20, wherein the compound of Formula (IV) is: 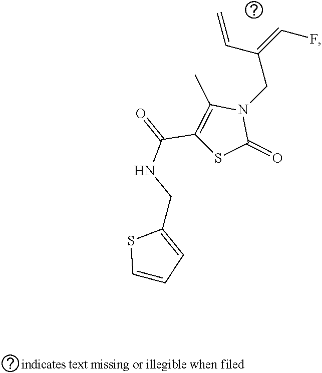 [SMILES: C=C/C(=C/F)Cn1c(C)c(C(=O)NCc2cccs2)sc1=O]
or a pharmaceutically acceptable salt thereof.

23. The method of any one of claims 14-20, wherein the compound of Formula (IV) is: 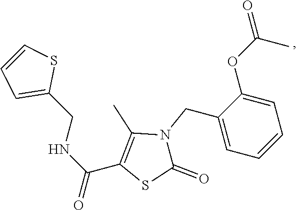 [SMILES: CC(=O)Oc1ccccc1Cn1c(C)c(C(=O)NCc2cccs2)sc1=O]
or a pharmaceutically acceptable salt thereof.

24. The method of any one of claims 14-20, wherein the compound of Formula (IV) is: 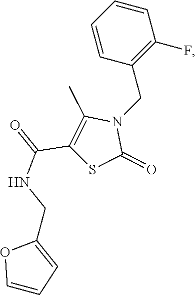 [SMILES: Cc1c(C(=O)NCc2ccco2)sc(=O)n1Cc1ccccc1F]
or a pharmaceutically acceptable salt thereof.

25. A method of treating a disease or disorder associated with inhibition of iRhom2/ADAM17 activity, said method comprising administering to a patient in need thereof a therapeutically effective amount of a compound of Formula (I): 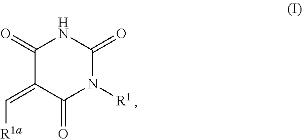
or a pharmaceutically acceptable salt thereof, wherein:
cycloalkyl;
R^{1a} is C₆₋₁₀ aryl, 5-10 membered heteroaryl, 4-10 membered heterocycloalkyl, or C₃₋₁₀ cycloalkyl;
wherein each R¹ or R^{1a} is optionally substituted with 1, 2 or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

26. The method of claim 25, wherein R¹ is C₆₋₁₀ aryl optionally substituted with 1, 2 or 3 substituents selected from the group consisting of halo and C₁₋₆ alkyl.

27. The method of claim 25 or 26, wherein R¹ is

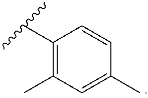 [SMILES: *c1ccc(C)cc1C]

28. The method of any one of claims 25-27, wherein R^{1a} is a 5-10 membered heteroaryl optionally substituted with 1, 2 or 3 substituents selected from the group consisting of halo, C₁₋₆ alkyl, C₁₋₄ alkyl(NHC(O)C₁₋₄ alkyl), C₁₋₄ alkyl(C(O)C₁₋₄ alkyl), C₁₋₄ alkyl(C(O)NHC₁₋₄ alkyl), C₁₋₄ alkyl(OC(O)C₁₋₄ alkyl) and C₁₋₄ alkyl((O)COC₁₋₄ alkyl),

29. The method of any one of claims 25-28, wherein R^{1a} is indole optionally substituted with 1, 2 or 3 substituents selected from the group consisting of halo, C₁₋₆ alkyl, C₁₋₄ alkyl(NHC(O)C₁₋₄ alkyl), C₁₋₄ alkyl(C(O)C₁₋₄ alkyl), C₁₋₄ alkyl(C(O)NHC₁₋₄ alkyl), C₁₋₄ alkyl(OC(O)C₁₋₄ alkyl) and C₁₋₄ alkyl((O)COC₁₋₄ alkyl).

30. The method of any one of claims 25-29, wherein R^{1a} is

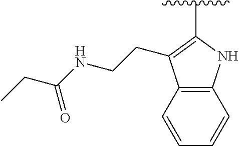 [SMILES: *c1[nH]c2ccccc2c1CCNC(=O)CC]

31. The method of any one of claims 25-30, wherein the compound of Formula (I) is: 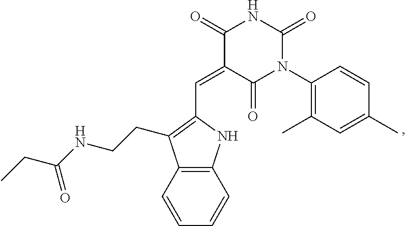 [SMILES: CCC(=O)NCCc1c(/C=C2/C(=O)NC(=O)N(c3ccc(C)cc3C)C2=O)[nH]c2ccccc12]
or a pharmaceutically acceptable salt thereof.

32. A method of treating a disease or disorder associated with inhibition of iRhom2/ADAM17 activity, said method comprising administering to a patient in need thereof a therapeutically effective amount of a compound of Formula (II): 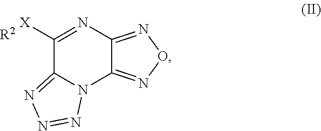
or a pharmaceutically acceptable salt thereof; wherein:
X is NH, N(C₁₋₄alkyl), O, S, S(O), S(O)₂, C(O), CH₂, CH(halo), C(halo)₂, CH(C₁₋₄alkyl), or C(C₁₋₄alkyl)₂;
R² is C₆₋₁₀ aryl, 5-10 membered heteroaryl, 4-10 membered heterocycloalkyl, or C₃₋₁₀ cycloalkyl;
wherein each R² is optionally substituted with 1, 2 or 3 substituents selected from the group consisting of halo, C₁₋₆ alkyl, C₁₋₆ haloalkyl, C₀₋₄ alkyl(C₃₋₆ cycloalkyl), C₁₋₆ alkoxy, C₀₋₆ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄ alkyl)), —C₀₋₄ alkyl(NHC(O)NH₂), —C₀₋₄ alkyl(NHC(O)NH(C₁₋₄ alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

33. The method of claim 32, wherein X is NH.

34. The method of claim 32 or 33, wherein R² is C₆₋₁₀ aryl optionally substituted with 1, 2 or 3 substituents selected from the group consisting of halo, C₁₋₆ alkyl, —C(O)(C₁₋₄ alkyl), —C(O)NH₂, —C(O)NH(C₁₋₄ alkyl), —C(O)N(C₁₋₄ alkyl)₂, —C(O)O(C₁₋₄ alkyl), S(O)(C₁₋₄ alkyl), —S(O)NH₂, —S(O)NH(C₁₋₄ alkyl), —S(O)N(C₁₋₄ alkyl)₂, —S(O)₂(C₁₋₄ alkyl), —S(O)₂NH₂, —S(O)₂NH(C₁₋₄ alkyl), and —S(O)₂N(C₁₋₄ alkyl)₂.

35. The method of any one of claims 32-34, wherein R² is

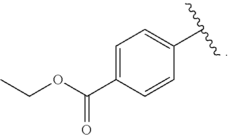 [SMILES: *c1ccc(C(=O)OCC)cc1]

36. The method of any one of claims 32-35, wherein the compound of Formula (II) is: 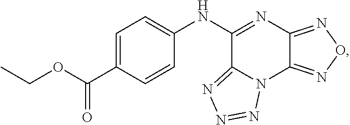 [SMILES: CCOC(=O)c1ccc(Nc2nc3nonc3n3nnnc23)cc1]
or a pharmaceutically acceptable salt thereof.

37. A method of treating a disease or disorder associated with inhibition of iRhom2/ADAM17 activity, said method comprising administering to a patient in need thereof a therapeutically effective amount of a compound of Formula (III): 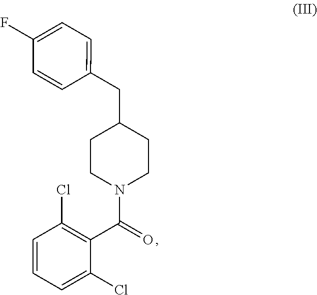 [SMILES: O=C(c1c(Cl)cccc1Cl)N1CCC(Cc2ccc(F)cc2)CC1]
or a pharmaceutically acceptable salt thereof.

38. A method of treating a disease or disorder associated with inhibition of iRhom2/ADAM17 activity, said method comprising administering to a patient in need thereof a therapeutically effective amount of a compound of Formula (IV): 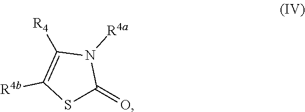
or a pharmaceutically acceptable salt thereof; wherein:
R⁴ is H, C₁₋₄ haloalkyl, or C₁₋₄ alkyl;
R^{4a} is —C₀₋₆ alkyl(C₆₋₁₀ aryl), —C₀₋₆ alkyl(5-10 membered heteroaryl), —C₀₋₆ alkyl(C₃₋₁₀cycloalkyl), or —C₀₋₆ alkyl(4-10 membered heterocycloalkyl);
R^{4b} is —C(O)—C₀₋₄alkyl(C₆₋₁₀ aryl), —C(O)—C₀₋₄alkyl(5-10 membered heteroaryl), —C(O)—C₀₋₄alkyl(C₃₋₁₀cycloalkyl), —C(O)—C₀₋₄alkyl(4-10 membered heterocycloalkyl), —NHC(O)—C₀₋₄alkyl(C₆₋₁₀ aryl), —NHC(O)—C₀₋₄alkyl(5-10 membered heteroaryl), —NHC(O)—C₀₋₄alkyl(C₃₋₁₀ cycloalkyl), —NHC(O)—C₀₋₄alkyl(4-10 membered heterocycloalkyl), —C(O)OC₀₋₄alkyl(C₆₋₁₀ aryl), —C(O)OC₀₋₄alkyl(5-10 membered heteroaryl), —C(O)OC₀₋₄alkyl(C₃₋₁₀ cycloalkyl), —C(O)OC₀₋₄alkyl(4-10 membered heterocycloalkyl), —C(O)NH—C₀₋₄alkyl(C₆₋₁₀ aryl), —C(O)NH—C₀₋₄alkyl(5-10 membered heteroaryl), —C(O)NH—C₀₋₄alkyl(C₃₋₁₀ cycloalkyl), —C(O)NH—C₀₋₄alkyl(4-10 membered heterocycloalkyl), —C(O)N(C₁₋₆ alkyl)(C₀₋₄alkyl-C₆₋₁₀ aryl), —C(O)N(C₁₋₆ alkyl)(C₀₋₄alkyl-(5-10 membered heteroaryl)), —C(O)N(C₁₋₆ alkyl)(C₀₋₄alkyl-C₃₋₁₀cycloalkyl), or —C(O)N(C₁₋₆ alkyl)(C₀₋₄alkyl-(4-10 membered heterocycloalkyl));
the group consisting of halo, C₁₋₆ alkyl, C₁₋₆ haloalkyl, C₀₋₄ alkyl(C₃₋₆cycloalkyl), C₁₋₆alkoxy, C₀₋₆ alkyl(CN), C₀₋₆ alkyl(OH), C₀₋₆ alkyl(NO₂), C₀₋₆ alkyl(NH₂), —C₀₋₄ alkyl(NH(C₁₋₄ alkyl)), —C₀₋₄ alkyl(N(C₁₋₄ alkyl)₂), methylenedioxy, —C₀₋₄ alkyl(S(C₁₋₄ alkyl)), —C₀₋₄ alkyl(C(O)(C₁₋₄ alkyl)), —C₀₋₄ alkyl(C(O)NH₂), —C₀₋₄ alkyl(C(O)NH(C₁₋₄ alkyl)), —C₀₋₄ alkyl(C(O)N(C₁₋₄ alkyl)₂), —C₀₋₄ alkyl(C(O)O(C₁₋₄ alkyl)), —C₀₋₄ alkyl(OC(O)(C₁₋₄ alkyl)), —C₀₋₄ alkyl(OC(O)NH₂), —C₀₋₄ alkyl (OC(O)NH(C₁₋₄ alkyl)), —C₀₋₄ alkyl(OC(O)N(C₁₋₄ alkyl)₂), —C₀₋₄ alkyl(NHC(O)(C₁₋₄ alkyl)), —C₀₋₄ alkyl(NHC(O)O(C₁₋₄alkyl)), —C₀₋₄ alkyl(NHC(O)NH₂), —C₀₋₄ alkyl(NHC(O)NH(C₁₋₄alkyl)), —C₀₋₄ alkyl(NHC(O)N(C₁₋₄ alkyl)₂), —C₀₋₄ alkyl(NHS(O)(C₁₋₄ alkyl)), —C₀₋₄ alkyl(NHS(O)₂(C₁₋₄ alkyl)), —C₀₋₄ alkyl(NHS(O)₂NH₂), —C₀₋₄ alkyl(NHS(O)₂NH(C₁₋₄ alkyl)), —C₀₋₄ alkyl(NHS(O)₂N(C₁₋₄ alkyl)₂), —C₀₋₄ alkyl(S(O)(C₁₋₄ alkyl)), —C₀₋₄ alkyl(S(O)NH₂), —C₀₋₄ alkyl(S(O)NH(C₁₋₄ alkyl)), —C₀₋₄ alkyl(S(O)N(C₁₋₄ alkyl)₂), —C₀₋₄ alkyl(S(O)₂(C₁₋₄ alkyl)), —C₀₋₄ alkyl(S(O)₂NH₂), —C₀₋₄ alkyl(S(O)₂NH(C₁₋₄ alkyl)), —C₀₋₄ alkyl(S(O)₂N(C₁₋₄ alkyl)₂), —C₀₋₄ alkyl(NHC(O)C₆₋₁₀ aryl), —C₀₋₄ alkyl(NHC(O)-(5-10 membered heteroaryl)), —C₀₋₄ alkyl(NHC(O)C₃₋₁₀ cycloalkyl), and —C₀₋₄ alkyl(NHC(O)-(4-10 membered heterocycloalkyl)).

39. The method of claim 38, wherein R⁴ is methyl.

40. The method of claim 38 or 39, wherein R^{4a} is —C₁₋₆ alkyl(C₆₋₁₀ aryl) optionally substituted with 1, 2, or 3 substituents selected from the group consisting of halo and —OC(O)(C₁₋₄ alkyl).

41. The method of any one of claim 38-40, wherein R^{4a} is selected from the group consisting of

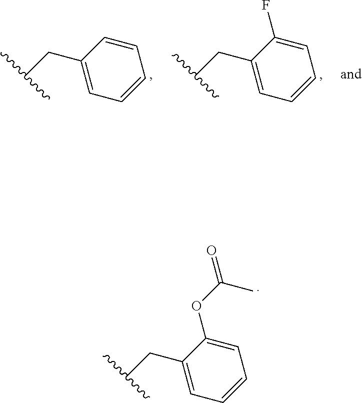 [SMILES: *Cc1ccccc1 ; *Cc1ccccc1F ; *Cc1ccccc1OC(C)=O]

42. The method of any one of claims 38-41, wherein R^{4b} is —C(O)NH—C₁₋₄alkyl(C₆₋₁₀ aryl), —C(O)NH—C₁₋₄alkyl(5-10 membered heteroaryl), —C(O)NH—C₁₋₄alkyl(C₃₋₁₀cycloalkyl), —C(O)NH—C₁₋₄alkyl(4-10 membered heterocycloalkyl), —C(O)N(C₁₋₆ alkyl)(C₁₋₄alkyl-C₆₋₁₀ aryl), —C(O)N(C₁₋ or —C(O)N(C₁₋₆ alkyl)(C₁₋₄alkyl-(4-10 membered heterocycloalkyl)).

43. The method of any one of claims 38-42, wherein R^{4b} is —C(O)NH—C₁₋₄alkyl(5-10 membered heteroaryl).

44. The method of any one of claims 38-43, wherein R^{4b} is selected from the group consisting of

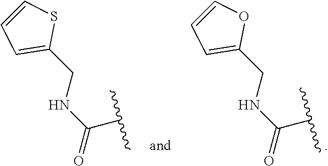 [SMILES: *C(=O)NCc1cccs1 ; *C(=O)NCc1ccco1]

45. The method of any one of claims 38-44, wherein the compound of Formula (IV) is: 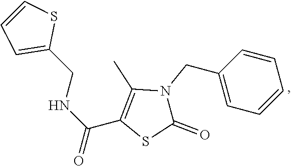 [SMILES: Cc1c(C(=O)NCc2cccs2)sc(=O)n1Cc1ccccc1]
or a pharmaceutically acceptable salt thereof.

46. The method of any one of claims 38-44, wherein the compound of Formula (IV) is: 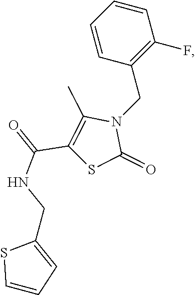 [SMILES: Cc1c(C(=O)NCc2cccs2)sc(=O)n1Cc1ccccc1F]
or a pharmaceutically acceptable salt thereof.

47. The method of any one of claims 38-44, wherein the compound of Formula (IV) is: 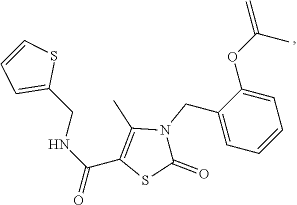 [SMILES: C=C(C)Oc1ccccc1Cn1c(C)c(C(=O)NCc2cccs2)sc1=O]
or a pharmaceutically acceptable salt thereof.

48. The method of any one of claims 38-44, wherein the compound of Formula (IV) is: 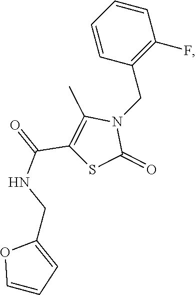 [SMILES: Cc1c(C(=O)NCc2ccco2)sc(=O)n1Cc1ccccc1F]
or a pharmaceutically acceptable salt thereof.

49. The method of any one of claims 25-48, wherein the disease or disorder is traumatic brain injury.

50. The method of any one of claims 25-48, wherein the disease or disorder is Alzheimer's Disease.

51. The method of any one of claims 25-48, wherein the disease or disorder is Hemorrhagic Stroke.

52. The method of any one of claims 25-48, wherein the disease or disorder is Hemophilic Arthropathy.

53. The method of any one of claims 25-48, wherein the disease or disorder is Cytokine Storm/Macrophase Activation Syndrome.
Arthritis.

55. The method of any one of claims 25-48, wherein the disease or disorder is Systemic Lupus Erythematosis-Glomerulonephritis.

56. The method of any of claims 25-55, wherein the compound is administered to the patient in a pharmaceutical composition comprising the compound and a pharmaceutically acceptable carrier or excipient.

57. The method of claim 56, wherein the pharmaceutical composition is in a pharmaceutical dosage form.

58. The method of claim 56 or 57, wherein the administration is parenteral.

59. The method of claim 56 or 57, wherein the administration is oral.

60. The method of claim 57 or 59, wherein the pharmaceutical dosage form is a tablet or a capsule.

61. The method of any one of claims 25-60, wherein the compound is administered to the patient at a daily dose in the range of about 50 mg/day to about 400 mg/day.

62. The method of any one of claims 25-60, wherein the compound is administered to the patient at a daily dose in the range of about 50 mg/day to about 300 mg/day, about 50 mg/day to about 300 mg/day, about 50 mg/day to about 200 mg/day, about 50 mg/day to about 100 mg/day, about 50 mg/day to about 75 mg/day, about 50 mg/day to about 60 mg/day, about 300 mg/day to about 400 mg/day, about 200 mg/day to about 400 mg/day, or about 100 mg/day to about 300 mg/day.
patient at a daily dose of about 50 mg/day.

64. The method of any one of claims 25-60, wherein the compound is administered to the patient at a daily dose of about 100 mg/day.

65. The method of any one of claims 25-60, wherein the compound is administered to the patient at a daily dose of about 200 mg/day.

66. The method of any one of claims 25-60, wherein the compound is administered to the patient at a daily dose of about 300 mg/day.

67. The method of any one of claims 25-60, wherein the compound is administered to the patient at a daily dose of about 400 mg/day.

68. The method of any one of claims 25-67, wherein the compound is administered to the patient in a single daily dose.

69. The method of any one of claims 25-67, wherein the daily dose of the compound is divided into multiple doses.

70. The method of any one of claims 25-69, wherein the compound is administered to the patient in combination with one or more additional therapeutic agents.


SEQUENCE LISTING (4 sequences)

SEQ ID NO: 1 - DNA, 2274 bp, Homo sapiens
  source 1..2274: mol_type = genomic DNA
atgctggggccctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctccgcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccggggtacctggagatctgcgggaatcctgtgactgataatgtaaaagacattacaaaactggtggcaaatcttccaaatgactatatgataaccctcaactatgtcgccgggatggatgttttgcctagtcattgttggctacgagatatggtaatacaattatcactcagcttgactactcttctggacaagttctcaaatatttctgaaggcttgagtaattactccatcatagacaaacttgggaaaatagtggatgacctcgtgttatgcatggaagaaaacgcaccgaagaatataaaagaatctccgaagaggccagaaactagatcctttactcctgaagaattctttagtattttcaatagatccattgatgcctttaaggactttatggtggcatctgacactagtgactgtgtgctctcttcaacattaggtcccgagaaagggaaagccgcaaaggcccctgaagactcgggcctacaatggacagccatggcattgccggctctcatttcgcttgtaattggctttgcttttggagccttatactggaagaagaaacagtcaagtcttacaagggcagttgaaaatatacagattaatgaagaggataatgagataagtatgttgcaacagaaagagagagaatttcaagaggttctagaagggcccgaacaaaaactcatctcagaagaggatctgaatagcgccgtcgaccatcatcatcatcatcattga

SEQ ID NO: 2 - protein, 757 aa, Homo sapiens
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGYLEICGNPVTDNVKDITKLVANLPNDYMITLNYVAGMDVLPSHCWLRDMVIQLSLSLTTLLDKFSNISEGLSNYSIIDKLGKIVDDLVLCMEENAPKNIKESPKRPETRSFTPEEFFSIFNRSIDAFKDFMVASDTSDCVLSSTLGPEKGKAAKAPEDSGLQWTAMALPALISLVIGFAFGALYWKKKQSSLTRAVENIQINEEDNEISMLQQKEREFQEVLEGPEQKLISEEDLNSAVDHHHHHH

SEQ ID NO: 3 - DNA, 1887 bp, Homo sapiens
  source 1..1887: mol_type = genomic DNA
atgctggggccctgcatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctccgcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccggggtacctggactgcccagattcccacactcagttctgcttccatggaacctgcaggtttttggtgcaggaggacaagccagcatgtgtctgccattctgggtacgttggtgcacgctgtgagcatgcggacctcctggccgtggtggctgccagccagaagaagcaggccatcaccgccttggtggtggtctccatcgtggccctggctgtccttatcatcacatgtgtgctgatacactgctgccaggtccgaaaacactgtgagtggtgccgggccctcatctgccggcacgagaagcccagcgccctcctgaagggaagaaccgcttgctgccactcagaaacagtggtctga

SEQ ID NO: 4 - protein, 628 aa, Homo sapiens
MLGPCMLLLLLLLGLRLQLSLGIIPVEEENPDFWNREAAEALGAAKKLQPAQTAAKNLIIFLGDGMGVSTVTAARILKGQKKDKLGPEIPLAMDRFPYVALSKTYNVDKHVPDSGATATAYLCGVKGNFQTIGLSAAARFNQCNTTRGNEVISVMNRAKKAGKSVGVVTTTRVQHASPAGTYAHTVNRNWYSDADVPASARQEGCQDIATQLISNMDIDVILGGGRKYMFRMGTPDPEYPDDYSQGGTRLDGKNLVQEWLAKRQGARYVWNRTELMQASLDPSVTHLMGLFEPGDMKYEIHRDSTLDPSLMEMTEAALRLLSRNPRGFFLFVEGGRIDHGHHESRAYRALTETIMFDDAIERAGQLTSEEDTLSLVTADHSHVFSFGGYPLRGSSIFGLAPGKARDRKAYTVLLYGNGPGYVLKDGARPDVTESESGSPEYRQQSAVPLDEETHAGEDVAVFARGPQAHLVHGVQEQTFIAHVMAFAACLEPYTACDLAPPAGTTDAAHPGYLDCPDSHTQFCFHGTCRFLVQEDKPACVCHSGYVGARCEHADLLAVVAASQKKQAITALVVVSIVALAVLIITCVLIHCCQVRKHCEWCRALICRHEKPSALLKGRTACCHSETVV